(19) United States
(12) Patent Application Publication
(10) Pub. No.: US 2026/0258106 A1
(43) Pub. Date: Sep. 3, 2026

(54) IL-21 POLYPEPTIDES AND METHODS OF USE

(71) Applicant: Beijing neoX Biotech Limited, Beijing (CN)

(72) Inventors: Puwei Yuan, Beijing (CN); Fei Zhang, Beijing (CN); Junli Liu, Beijing (CN); Mingchen Chen, Beijing (CN); Chengcheng Guan, Beijing (CN); Xin Zheng, Beijing (CN); Hang Chen, Beijing (CN); Fan Liu, Beijing (CN)

(21) Appl. No.: 18/878,540

(22) PCT Filed: Jun. 28, 2023
(86) PCT No.: PCT/CN2023/103239

§ 371 Date: Dec. 23, 2024

(30) Foreign Application Priority Data
Jun. 29, 2022 (WO) PCT/CN2022/102455
Dec. 7, 2022 (WO) PCT/CN2022/137188
Jun. 6, 2023 (WO) PCT/CN2023/098625

(51) Int. Cl. C07K 14/54; A61K 38/00; A61K 47/68; A61P 35/00; C07K 16/28

(52) U.S. Cl. CPC C07K 14/54; A61K 47/68; A61P 35/00; C07K 16/2818; A61K 38/00; C07K 2319/02; C07K 2319/30

(57) ABSTRACT

Provided herein are various polypeptides comprising an IL-21 variant. Further provided herein are conjugates comprising the polypeptide and at least one additional moiety. Further provided herein are related nucleic acid molecules, vectors, transformed or host cells pharmaceutical compositions and kits. Also provided herein are methods of preparing the polypeptides or the conjugates described herein and treatment methods of using such.

### BACKGROUND

Interleukin-21 (IL-21) has a four-helix bundle structure and exists as a monomer. In humans, two isoforms of IL-21 are known, each of which are derived from a precursor molecule. The first IL-21 isoform comprises 162 amino acids (aa) including a signal peptide of 29 aa long. The second IL-21 isoform comprises 153 aa, including a signal peptide of 29 aa long. The amino acid sequences of isoform 1 and isoform 2 are provided herein as SEQ ID NOs: 3 and 4, respectively. IL-21 sometimes refers to a fragment of the full length IL-21 isoform 1 comprises a signal peptide and a mature polypeptide of 133 amino acids (SEQ ID NO: 1) or a fragment of the full length IL-21 isoform 2 comprises a signal peptide and a mature polypeptide of 124 amino acids (SEQ ID NO: 2). IL-21 is mainly expressed in T follicular helper (Tfh) cells, Th2 and Th17 cell subsets, and NK cells. IL-21 possesses anti-tumor effects by enhancing cytotoxicity of CD8⁺ T cells and NK cells and stimulating their maturation (see e.g., Vidard L et al., J Immunol. 2019; 203(3):676-685). The low abundance of IL-21 accounts in part for the impaired activity of cytotoxic T lymphocytes in the tumor microenvironment.

Since IL-21R is widely expressed on the surface of normal lymphoid tissues including spleen, thymus, lymph nodes, peripheral blood lymphocytes, T cells, B cells, and NK cells, wildtype IL-21 may cause systemic inflammatory effects in the related cancer therapies.

### SUMMARY

Addressing the need for IL-21-related therapeutics with better safety and stability profile, improved pharmacokinetics, and maximized pharmacodynamics profiles, and stronger tumor inhibition potency, provided herein are polypeptides comprising an IL-21 variant with an amino acid residue substitution at position P79 corresponding to a wildtype human IL-21. In some embodiments, the wildtype human IL-21 comprises a sequence as set forth in SEQ ID NO: 1 or 2.

In some embodiments, the amino acid residue substitution at position P79 is P79E or P79C. In some specific embodiments, the amino acid residue substitution at position P79 is P79E. In other specific embodiments, the amino acid residue substitution at position P79 is P79C, and the polypeptide further comprises one or more amino acid residue substitutions with cysteine in the region of positions 1-10 corresponding to the wildtype human IL-21. In some cases, the one or more amino acid residue substitutions are selected from the group consisting of R5C, H6C and R9C. In some cases, the cysteine residues of the one or more amino acid residue substitutions in the region of positions 1-10 form a disulfide linkage together with the cysteine residue at position P79.

In some embodiments, the polypeptide comprises one or more amino acid residue substitutions at positions selected from the group consisting of: R5, H6, R9, Q12, L13, D15, I16, S70, K72, K73, R76, and P78. In some embodiments, the polypeptide comprises one or more amino acid residue substitutions at positions selected from the group consisting of S70, K72, K73, and R76.

In specific embodiments, the polypeptide further comprises an amino acid residue substitution at position K73 corresponding to the wildtype human IL-21. In some cases, the amino acid residue substitution at position K73 is an aromatic amino acid. In specific cases, the amino acid residue substitution at position K73 is K73Y or K73F.

In specific embodiments, the polypeptide further comprises an amino acid residue substitution at position S70 corresponding to the wildtype human IL-21. In some cases, the amino acid residue substitution at S70 is S70L or S70A.

In specific embodiments, the polypeptide further comprises an amino acid residue substitution at position R76 corresponding to the wildtype human IL-21. In some cases, the amino acid residue substitution at R76 is R76F.

In specific embodiments, the polypeptide further comprises an amino acid residue substitution at position K72 corresponding to the wildtype human IL-21. In some cases, the amino acid residue substitution at K72 is a non-aromatic amino acid comprising a side chain hydroxyl. In some specific cases, the amino acid residue substitution at K72 is K72S.

In specific embodiments, the polypeptide further comprises an amino acid residue substitution at position Q12 corresponding to the wildtype human IL-21. In some cases, the amino acid residue substitution at Q12 is Q12W.

In specific embodiments, the polypeptide further comprises an amino acid residue substitution at position L13 corresponding to the wildtype human IL-21. In some cases, the amino acid residue substitution at L13 is L13A.

In specific embodiments, the polypeptide further comprises an amino acid residue substitution at position D15 corresponding to the wildtype human IL-21. In some cases, the amino acid residue substitution at D15 is D15L, D15K or D15R.

In specific embodiments, the polypeptide further comprises an amino acid residue substitution at position 116 corresponding to the wildtype human IL-21. In some cases, the amino acid residue substitution at 116 is 116R or 116W.

In specific embodiments, the polypeptide further comprises an amino acid residue substitution at position P78 corresponding to the wildtype human IL-21. In some cases, the amino acid residue substitution at P78 is P78G or P78E.

In some embodiments, the polypeptide further comprises amino acid residue mutations selected from the group consisting of: P79E; K73Y, P79E; K73F, P79E; S70L, K73F, P79E; S70L, K73Y, P79E; S70L, K72S, K73F, P79E; S70L, K72S, K73Y, P79E; D15K, S70L, K73F, P79E; D15R, S70L, K73F, P79E; I16W, S70L, K73F, P79E; D15L, S70L, K73F, P79E; L13A, S70L, K73F, P79E; D15R, R76F, P78E, P79E; D15L, I16R, S70L, K73Y, P79E; D15K, I16W, S70L, R76F, P79E; D15R, 116W, S70L, R76F, P79E; STNAGRRQKHR (SEQ ID NO: 71) substituted with GGGSEGGGS (SEQ ID NO: 72) (STNAGRRQKHR (SEQ ID NO: 71)/GGGSEGGGS (SEQ ID NO: 72)); P79E, STNAGRRQKHR (SEQ ID NO: 71)/GGGSEGGGS (SEQ ID NO: 72); K73Y, P79E, STNAGRRQKHR (SEQ ID NO: 71)/GGGSEGGGS (SEQ ID NO: 72); K73F, P79E, STNAGRRQKHR (SEQ ID NO: 71)/GGGSEGGGS (SEQ ID NO: 72); S70L, K73F, P79E, STNAGRRQKHR (SEQ ID NO: 71)/GGGSEGGGS (SEQ ID NO: 72); S70L, K73Y, P79E, STNAGRRQKHR (SEQ ID NO: 71)/GGGSEGGGS (SEQ ID NO: 72); D15K, S70L, K73F, P79E, STNAGRRQKHR (SEQ ID NO: 71)/GGGSEGGGS (SEQ ID NO: 72); D15R, S70L, K73F, P79E, STNAGRRQKHR (SEQ ID NO: 71)/GGGSEGGGS (SEQ ID NO: 72); I16W, S70L, K73F, P79E, STNAGRRQKHR (SEQ ID NO: 71)/GGGSEGGGS (SEQ ID NO: 72); S70L, K72S, K73F, P79E, STNAGRRQKHR (SEQ ID NO: 71)/GGGSEGGGS (SEQ ID NO: 72); S70L, K72S, K73Y, P79E, STNAGRRQKHR (SEQ ID NO: 71)/GGGSEGGGS (SEQ ID NO: 72); D15R, I16W, S70L, K73Y, P79E, STNAGRRQKHR (SEQ ID NO: 71)/GGGSEGGGS (SEQ ID NO: 72); and D15L, I16R, S70L, K73Y, P79E, STNAGRRQKHR (SEQ ID NO: 71)/GGGSEGGGS (SEQ ID NO: 72).

In some embodiments, the polypeptide further comprises amino acid residue mutations selected from the group consisting of: S70L, K73F, P79E; S70L, K72S, K73F, P79E; S70L, K73Y, P79E; and S70L, K72S, K73Y, P79E.

In some embodiments, the polypeptide further comprises amino acid residue mutations selected from the group consisting of: P79C; R5C, P79C; H6C, P79C; R9C, P79C; R5C, R76F, P79C; H6C, R76F, P79C; H6C, K73Y, P79C; H6C, K73F, P79C; H6C, S70L, K73Y, P79C; H6C, S70L, K73F, P79C; R9C, D15R, P78G, P79C; H6C, D15L, I16R, S70L, K73Y, P79C; R9C, D15L, 116R, S70L, K73Y, P79C; H6C, P79C, STNAGRRQKHR (SEQ ID NO: 71)/GGGSEGGGS (SEQ ID NO: 72); H6C, K73Y, P79C, STNAGRRQKHR (SEQ ID NO: 71)/GGGSEGGGS (SEQ ID NO: 72); H6C, K73F, P79C, STNAGRRQKHR (SEQ ID NO: 71)/GGGSEGGGS (SEQ ID NO: 72); and H6C, S70L, P79C, STNAGRRQKHR (SEQ ID NO: 71)/GGGSEGGGS (SEQ ID NO: 72).

In another aspect, provided herein are polypeptides comprising an IL-21 variant with an amino acid residue substitution at position S70 or K73 corresponding to a wildtype human IL- 21. In some embodiments, the wildtype human IL-21 comprises a sequence as set forth in SEQ ID NO: 1 or 2.

In some embodiments, the polypeptide comprises an amino acid residue substitution with an aromatic amino acid at position K73 corresponding to the wildtype human IL-21. In some cases, the amino acid residue substitution at position K73 is K73Y and K73F. In some embodiments, the polypeptide comprises an amino acid residue substitution at position S70. In some cases, the amino acid residue substitution at position S70 is S70L or S70A.

In some embodiments, the polypeptide comprises an amino acid residue substitution at position S70 (e.g., S70L or S70A) and an amino acid residue substitution at position K73 corresponding to the wildtype human IL-21. In specific embodiments, the polypeptide comprises an amino acid residue substitution with an aromatic amino acid at position K73 corresponding to the wildtype human IL-21. In specific cases, the amino acid residue substitution at position K73 is K73Y or K73F. In other embodiments, the polypeptide further comprises one, two, or three, or more amino acid residue substitutions at positions selected from the group consisting of: R5, H6, R9, Q12, L13, D15, I16, K72, R76, P78 and P79. In specific embodiments, the polypeptide further comprises one, two, or three, or four amino acid residue substitutions at positions selected from the group consisting of: Q12, D15, I16, and K72. In some cases, the polypeptide comprises an amino acid residue substitution selected from the group consisting of: 116R and I16W. In other cases, the polypeptide comprises an amino acid residue substitution selected from the group consisting of: D15L, D15K, and D15R. In yet other cases, the polypeptide comprises an amino acid residue substitution selected from the group consisting of: K72S and Q12W.

In some embodiments, the polypeptide comprises an amino acid residue substitution at position S70 (e.g., S70L or S70A) and an amino acid residue substitution at position R76 corresponding to the wildtype human IL-21. In some cases, the amino acid residue substitution at position R76 is R76F. In some cases, the polypeptide further comprises one, two, or three, or more amino acid residue substitutions at positions selected from the group consisting of: R5, H6, R9, Q12, L13, D15, I16, K72, K73, P78 and P79. In some cases, the polypeptide further comprises one, two, or three, or four amino acid residue substitutions at positions selected from the group consisting of: Q12, D15, I16, and K72. In some cases, the polypeptide comprises an amino acid residue substitution selected from the group consisting of: 116R and 116W. In other cases, the polypeptide comprises an amino acid residue substitution selected from the group consisting of D15L, D15K, and D15R. In yet other cases, the polypeptide comprises an amino acid residue substitution selected from the group consisting of: K72S and Q12W.

In some embodiments, the polypeptide comprises amino acid residue mutations selected from the group consisting of: K73Y; K73F; S70L, K73Y; S70L, K73F; S70L, K72S, K73Y; S70A, K72S, K73F; Q12W, S70A, R76F; D15K, S70A, R76F; D15L, S70A, R76F; D15R, S70A, R76F; I16R, S70L, K73Y; D15L, I16R, S70L, K73Y; D15K, I16W, S70L, K73Y; D15K, I16W, S70L, R76F; D15R, I16W, S70L, R76F; I16W, S70L, K72S, K73F; D15R, I16W, S70L, K73Y; and D15K, I16W, S70L, K72S, K73Y.

In some embodiments, the amino acid segment of STNAGRRQKHR (SEQ ID NO: 71) at positions 80-90 corresponding to the wildtype human IL-21 is substituted with a short peptide linker. In some embodiments, the length of the short peptide linker is from 4 to 10 amino acid residues, such as 4, 5, 6, 7, 8, 9, or 10 amino acid residues. In some embodiments, the length of the short peptide linker is from 5 to 9 amino acid residues. In some embodiments, the length of the short peptide linker is from 7 to 9 amino acid residues. In further embodiments, the short peptide linker comprises one or more Gly-Ser units, and optionally one glutamic acid residue. In specific embodiments, the short peptide linker comprises the glutamic acid residue, and the glutamic acid residue is in the middle of the short peptide linker. In specific embodiments, the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

In some embodiments, the polypeptide exhibits a lower binding affinity for IL-21 receptor (IL-21R) than the wildtype human IL-21. In specific embodiments, the polypeptide exhibits 2 to 1000 fold reduction in binding affinity for IL-21R as compared to the wildtype human IL-21. In specific embodiments, the polypeptide has a higher K_{D} value for IL-21R as compared to the wildtype human IL-21.

In another aspect, provided herein are polypeptides comprising an interleukin-21 (IL-21) variant, wherein the amino acid segment of STNAGRRQKHR (SEQ ID NO: 71) at positions 80-90 corresponding to a wildtype human IL-21 is substituted with a short peptide linker. In some embodiments, the length of the short peptide linker is from 4 to 10 or from 5 to 9 amino acid residues. In some cases, the short peptide linker comprises one or more Gly-Ser units, and optionally one glutamic acid residue. In specific cases, the short peptide linker comprises the glutamic acid residue, and the glutamic acid residue is in the middle of the short peptide linker. In specific cases, the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

In some embodiments, the polypeptide comprises one or more amino acid residue substitutions at positions selected from the group consisting of R5, H6, R9, Q12, L13, D15, I16, S70, K72, K73, R76, P78, and P79. In specific embodiments, the polypeptide comprises the amino acid residue substitution at position R5, and optionally the substitution is R5C. In specific embodiments, the polypeptide comprises the amino acid residue substitution at position H6, and optionally the substitution is H6C. In specific embodiments, the polypeptide comprises the amino acid residue substitution at position R9, and optionally the substitution is R9C. In specific embodiments, the polypeptide comprises the amino acid residue substitution at position Q12, and optionally the substitution is Q12W. In specific embodiments, the polypeptide comprises the amino acid residue substitution at position L13, and optionally the substitution is L13A. In specific embodiments, the polypeptide comprises the amino acid residue substitution at position D15, and optionally the substitution is D15L, D15K, or D15R. In specific embodiments, the polypeptide comprises the amino acid residue substitution at position 116, and optionally the substitution is 116R or 116W. In specific embodiments, the polypeptide comprises the amino acid residue substitution at position S70, and optionally the substitution is S70L or S70A. In specific embodiments, the polypeptide comprises the amino acid residue substitution at position K72, and optionally the substitution is K72S. In specific embodiments, the polypeptide comprises the amino acid residue substitution at position K73, and optionally the substitution is K73Y or K73F. In specific embodiments, the polypeptide comprises the amino acid residue substitution at position R76, and optionally the substitution is R76F. In specific embodiments, the polypeptide comprises the amino acid residue substitution at position P78, and optionally the substitution is P78G or P78E. In specific embodiments, the polypeptide comprises the amino acid residue substitution at position P79, and optionally the substitution is P79E or P79C.

In some embodiments, the polypeptide exhibits an improved thermostability as compared to the wildtype human IL-21. In some embodiments, the polypeptide exhibits a lower binding affinity for IL-21 receptor (IL-21R) than wild-type IL-21. In some embodiments, the polypeptide exhibits 2 to 1000 fold reduction in binding affinity for IL-21R as compared to wild-type IL-21. In some embodiments, the polypeptide has a higher K_{D} value for IL-21R as compared to wild-type IL-21. In some embodiments, the polypeptide exhibits an improved efficacy in inhibiting tumor growth and reducing tumor volume as compared to the wildtype human IL-21. In some embodiments, the polypeptide exhibits an improved efficacy and/or achieves synergistic effect in treating tumor when it is fused with or administered in combination with the at least one additional moiety/agent as disclosed herein, such as anti-PD1 antibody.

In another aspect, provided herein are polypeptides comprising an interleukin-21 (IL-21) variant comprising a wildtype human IL-21 amino acid sequence with at least one mutation selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C. In some embodiments, the wildtype human IL-21 comprises a sequence as set forth in SEQ ID NO: 1 or 2.

In some embodiments, the amino acid segment of STNAGRRQKHR (SEQ ID NO: 71) at positions 80-90 corresponding to the wildtype human IL-21 is substituted with a short peptide linker. In some embodiments, the length of the short peptide linker is from 4 to 10 amino acid residues, such as 4, 5, 6, 7, 8, 9 or 10 amino acid residues. In some embodiments, the length of the short peptide linker is from 5 to 9 amino acid residues. In some embodiments, the length of the short peptide linker is from 7 to 9 amino acid residues. In some embodiments, the short peptide linker comprises one or more Gly-Ser units, and optionally one glutamic acid residue. In some embodiments, the short peptide linker comprises the glutamic acid residue, and the glutamic acid residue is in the middle of the short peptide linker. In some embodiments, the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

In some specific embodiments, the polypeptide comprises a sequence as set forth in any one of SEQ ID Nos. 5-62. In some specific embodiments, the polypeptide consists of a sequence as set forth in any one of SEQ ID Nos. 5-62.

In some embodiments, the polypeptide exhibits a lower binding affinity for IL-21 receptor (IL-21R) than the wildtype human IL-21. In some embodiments, the polypeptide exhibits 2 to 1000 fold reduction in binding affinity for IL-21R as compared to the wildtype human IL-21. In some embodiments, the polypeptide has a higher K_{D} value for IL-21R as compared to the wildtype human IL-21.

In another aspect, provided herein are conjugates comprising the polypeptide disclosed herein and at least one additional moiety. In some embodiments, the at least one additional moiety comprises a crystallizable fragment (Fe) domain of an antibody. In some specific embodiments, the antibody is IgG, IgA, IgD, IgM, or IgE. In some specific embodiments, the IgG is IgG1, IgG2, IgG3, or IgG4.

In some embodiments, the at least one additional moiety comprises an antigen-binding molecule. In specific embodiments, the antigen-binding molecule is an antibody or an antigen binding fragment thereof. In specific embodiments, the antigen-binding molecule is a multi-specific antigen-binding molecule.

In some embodiments, the antigen-binding molecule targets a tumor cell or an immune cell. In some embodiments, the antigen is a tumor-associated antigen. In other embodiments, the antigen is an immune-checkpoint antigen or an immune-checkpoint-associated antigen. In specific embodiments, the antigen is involved in an immune checkpoint pathway. In specific embodiments, the antigen is selected from the group consisting of: PD-1, PD-L1, TIGIT, CTLA-4, PD-L2, B7-H3, B7-H4, BTLA, LAG3, CD112, CD112R, CD96, TIM-3, CD47, and CEACAM1. In specific embodiments, the antigen is a co-stimulatory immune checkpoint target. In specific embodiments, the antigen is selected from the group consisting of: CD155, ICOS, OX40, CD137, CD137L, CD27, CD28, and GITR.

In some embodiments, the at least one additional moiety is attached to C-terminus and/or N-terminus of the IL-21 variant. In some embodiments, the at least one additional moiety is attached to the IL-21 variant directly or via a linker.

In another aspect, provided herein are nucleic acid molecules encoding the polypeptides disclosed herein or the conjugates disclosed herein. In some embodiments, the nucleic acid molecule is a DNA molecule. In some embodiments, the nucleic acid molecule is an RNA molecule. In some embodiments, the nucleic acid molecule is an mRNA molecule. In another aspect, provided herein are vectors comprising the nucleic acid molecules provided herein. In some embodiments, the vectors comprise a viral vector. In another aspect, provided herein are transformed or host cells that expresses the polypeptides disclosed herein or the conjugate disclosed herein. In another aspect, provided herein are pharmaceutical compositions comprising the polypeptides disclosed herein or the conjugates disclosed herein, and pharmaceutically acceptable carriers. In another aspect, provided herein are kits comprising the polypeptides, the conjugates, the nucleic acid molecules, the vectors, the transformed or host cells, or the pharmaceutical compositions, a combination thereof, and containers.

In another aspect, provided herein are methods of preparing the polypeptides described herein or the conjugates described herein, comprising: (a) constructing the nucleic acid molecules and the vectors described herein; (b) culturing the transformed or host cells described herein; and (c) harvesting the polypeptides from the transformed or host cells.

In another aspect, provided herein are methods of treating a subject in need thereof, comprising administering to the subject the pharmaceutical compositions described herein in an amount effective to treat the subject. In some embodiments, the subject has a solid tumor.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The application contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows a diagram of an expression vector containing wildtype IL-21-Fc.

FIGS. 2A-2F illustrate results of running wildtype IL-21-Fc and IL-21 variant-Fe proteins on SDS-PAGE protein gels. FIG. 2A shows results of running wildtype IL-21-Fe on SDS-PAGE protein gels. FIG. 2B shows results of running m92-Fc on SDS-PAGE protein gels.

FIG. 2C shows results of running m98-Fc on SDS-PAGE protein gels. FIG. 2D shows results of running m99-Fc on SDS-PAGE protein gels. FIG. 2E shows results of running m100-Fc on SDS-PAGE protein gels. FIG. 2F shows results of running m133-Fc on SDS-PAGE protein gels.

FIGS. 3A-3F illustrate results of detecting wildtype IL-21-Fe and IL-21 variant-Fc proteins on size exclusion chromatography (SEC). FIG. 3A shows results of detecting wildtype IL-21-Fe on SEC. FIG. 3B shows results of detecting m92-Fc on SEC. FIG. 3C shows results of detecting m98-Fc on SEC. FIG. 3D shows results of detecting m99-Fc on SEC. FIG. 3E shows results of detecting m100-Fc on SEC. FIG. 3F shows results of detecting m133-Fc on SEC.

FIGS. 4A-4F illustrate the BLI affinity test results ofwildtype IL-21-Fc and IL-21 variant-Fc proteins. FIG. 4A shows the BLI affinity test results of wildtype IL-21-Fc. FIG. 4B shows the BLI affinity test results of m92-Fc. FIG. 4C shows the BLI affinity test results of m98-Fe. FIG. 4D shows the BLI affinity test results of m99-Fc. FIG. 4E shows the BLI affinity test results of m100-Fc. FIG. 4F shows results the BLI affinity test results of m133-Fc.

FIG. 5 shows STAT3 phosphorylation in HuT78 cells by wildtype IL-21-Fe or IL-21 mutant-Fc proteins.

FIG. 6 shows results of in vivo efficacy study investigating tumor inhibition efficacy of anti-mPD1 antibody_IL21m fusion proteins in tumor-bearing mice.

FIGS. 7A-7B illustrate results of running wildtype IL-21-IgG4 Fc and IL-21 variant-IgG4 Fc proteins on SDS-PAGE protein gels. FIG. 7A shows results of running wildtype IL-21-IgG4 Fc on SDS-PAGE protein gels. FIG. 7B shows results of running m98-21-IgG4 Fc on SDS-PAGE protein gels.

FIGS. 8A-8B illustrate results of detecting wildtype IL-21-IgG4 Fc and IL-21 variant-IgG4 Fc proteins on size exclusion chromatography (SEC). FIG. 8A shows results of detecting wildtype IL-21-IgG4 Fc on SEC. FIG. 8B shows results of detecting m98-IgG4 Fc on SEC.

FIGS. 9A-9D illustrate results of running wildtype IL-21-His and IL-21 variant-His proteins on SDS-PAGE protein gels. FIG. 9A shows results of running wildtype IL-21-His on SDS-PAGE protein gels. FIG. 9B shows results of running m98-His on SDS-PAGE protein gels. FIG. 9C shows results of running m103-His on SDS-PAGE protein gels. FIG. 9D shows results of running m153-His on SDS-PAGE protein gels.

FIGS. 10A-10D illustrate results of running fusion protein of wildtype IL-21 or an IL-21 variant with anti-mouse PD1 antibody on SDS-PAGE protein gels. FIG. 10A shows results of running anti-mPD1 antibody—wildtype IL21 fusion protein on SDS-PAGE protein gels. FIG. 10B shows results of running anti-mPD1 antibody—IL21m98 fusion protein on SDS-PAGE protein gels. FIG. 10C shows results of running anti-mPD1 antibody—IL21m100 fusion protein on SDS-PAGE protein gels. FIG. 10D shows results of running anti-mPD1 antibody-IL21m153 fusion protein on SDS-PAGE protein gels.

FIG. 11 illustrates the change of tumor volume over time after treating tumor bearing mice with wildtype IL-21—IgG4 Fc or m98—IgG4 Fc proteins.

FIG. 12 illustrates the change of tumor volume over time after treating tumor bearing mice with anti-mPD1 antibody alone, m98—IgG4 Fc alone, or a combination of both.

FIG. 13 illustrate the tumor weight isolated from tumor bearing mice which were treated with anti-mPD1 antibody alone, m98—IgG4 Fc alone, or a combination of both.

### DETAILED DESCRIPTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus, the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the context of the present invention, when describing a specific combinational mutation, phrase “mutation A, mutation B, mutation C” means that these mutations A, B, and C are present at the same time in a single IL-21 variant. For example, a combinational mutation ‘S70L, K73Y, P79E′ means that mutations S70L, K73Y, and P79E are all present in a single IL-21 variant. Also as used in the context of the present invention, term “STNAGRRQKHR (SEQ ID NO: 71)/GGGSEGGGS (SEQ ID NO: 72)” refers to a mutation where the amino acid segment of STNAGRRQKHR (SEQ ID NO: 71) at positions 80-90 corresponding to the wildtype human IL-21 is substituted with GGGSEGGGS (SEQ ID NO: 72).

As used in the specification and appended claims, unless specified to the contrary, the following terms have the meaning indicated below.

“Wildtype” or “WT” or “wt” or “native” refers to an amino acid sequence that is found in nature, including allelic variations or natural isoforms. A wildtype protein or polypeptide has an amino acid sequence that has not been intentionally modified.

“Variant” or “mutant”, which can be used interchangeably, of an amino acid sequence (e.g., of a peptide, protein, or polypeptide) refers to amino acid insertion variants, amino acid addition variants, amino acid deletion variants, and/or amino acid substitution variants. Amino acid insertion variants are characterized by insertion of one or more amino acids in a particular amino acid sequence, e.g., a wildtype IL-21 sequence or a functional variant thereof. Amino acid addition variants include N- and/or C-terminal fusions of one or more amino acids. Amino acid deletion variants are characterized by removal of one or more amino acids from the sequence. The deletions may be in any position of the protein sequence. Amino acid deletion variants include deletion at the N- and/or C-terminus of the protein to produce N- and/or C-terminal truncation variants. Amino acid substitution variants are characterized by removal of one or more amino acids from the sequence, and insertion of one or more another amino acids in place of the original amino acids.

“IL-21 mutant” or “IL-21 mutein” or “IL-21 variant” or “IL-21 variant moiety” herein refers to a polypeptide in which one or more amino acids of the wildtype human IL-21 or a functional variant thereof are mutated to provide a mutant or variant of the wildtype human IL-21. In some embodiments, an IL-21 mutant has a sequence that is at least 80% identical to the sequence of wildtype human IL-21 (e.g., SEQ ID NO: 1 or 2). In some embodiments, an IL-21 mutant polypeptide provided herein comprises a signal peptide. In some specific embodiments, the signal peptide is a naturally accruing signal peptide of wildtype human IL-21. In some specific embodiments, the signal peptide is any signal peptide known in the field. In some embodiments, an IL-21 mutant polypeptide provided herein does not comprise a signal peptide.

“Natural amino acid” refers to an amino acid of the 20 naturally-occurring amino acids in protein. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are classified as aromatic amino acids.

“Unnatural amino acid” refers to an amino acid other than the 20 naturally-occurring amino acids in protein.

“Percentage identity” refers to a percentage of identical amino acid residues between two sequences being compared after an optimal alignment of sequences. An optimal alignment of sequences may be produced manually or by means of computer programs which use a sequence alignment algorithm (e.g., ClustalW, T-coffee, COBALT, BestFit, FASTA, BLASTP, BLASTN, and TFastA). Percentage identity can be calculated by determining the number of identical positions between the two sequences being compared, dividing this number by the number of positions compared, and multiplying the result obtained by 100 so as to obtain the percentage identity between the two sequences.

The term “subject” or “patient” encompasses mammals. Examples of mammals include, but are not limited to, any member of the mammalian class: humans, non-human primates, such as chimpanzees, and other apes and monkey species; farm animals, such as cattle, horses, sheep, goats, swine; domestic animals, such as rabbits, dogs, and cats; laboratory animals, including rodents, such as rats, mice and guinea pigs, and the like. In one aspect, the mammal is a human.

As used herein, “treatment” or “treating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### IL-21

IL-21 is a member of a large family of cytokines (IL-2, IL-4, IL-7, IL-9 and IL-15) whose receptors share a common receptor γ chain (γc). IL-21 is a pleiotropic type I cytokine that is produced mainly by T cells and natural killer T (NKT) cells. This cytokine has diverse effects on a broad range of cell types including, but not limited to, CD4+ and CD8+ T cells, B cells, macrophages, monocytes, and dendritic cells (DCs). Specifically, IL-21 drives B cell differentiation into plasma cells, regulates immunoglobulin production, controls the proliferation and/or effector function of both CD4+ and CD8+ T cells, limits the differentiation of Tregs and can stimulate epithelial cells and fibroblasts to produce inflammatory mediators (see e.g., Leonarda and Wan, F1000Res. 2016; 5: F1000 Faculty Rev-224; Stolfi et al, Oncoimmunology. 2012 May 1; 1(3): 351-354).

The IL-21 receptor (IL-21R) forms a heterodimeric receptor complex with a common ye, which is also a subunit of the receptors for IL-2, IL-4, IL-7, IL-9, and IL-15. The IL-21R moiety is the ligand recognition binding site, and ye is the signal transduction unit. IL-21R subunits are mainly expressed on the surface of normal lymphoid tissues including spleen, thymus, lymph nodes, peripheral blood lymphocytes, T cells, B cells, and NK cells. IL-21R acts through the JAK/STAT pathway, and activates its target genes via JAK1, JAK3, and a STAT3 dimer.

IL-21R transduces the growth-promoting signal of IL-21, which plays an important role in regulating the proliferation of B cells, promoting the proliferation and differentiation of multiple T cell subsets, regulating survival of NK cells, and improving the cytotoxicity activity of NK cells. Studies in IL-21R knockout mice have shown that its important roles in regulating the production of immunoglobulins. Furthermore, IL-21 has been reported to have potent anti-tumor effects due to its ability to expand the pool of cytotoxic CD8+ T cells, NK cells and NKT cells

Compared with the affinity of wildtype IL-21 protein and IL-21R, the affinity of IL-21 variant of the present disclosure to IL-21R is reduced. As such, in some embodiments, due to the reduced affinity of IL-21 variant described herein with IL-21R in normal lymphoid tissues, the polypeptides described herein result in less systemic toxicity. In some embodiments, the polypeptides of less systemic toxicity described herein is therefore with advantages on dosing strategy, therapeutic window when administered as single drug or in combination therapy, and also a more compatible candidate for conjugation with another functional moiety (such as antibodies, small molecule inhibitors, or nucleic acids) to form a bi- or multi-functional molecule for therapies.

In some embodiments, the thermal stability of the IL-21 mutants of the present disclosure is improved compared with the wildtype IL-21. In some embodiments, the pharmacodynamics of the IL-21 mutants of the present disclosure is improved. Therefore, the druggability of the IL-21 mutants is optimized compared with the wildtype IL-21.

### IL-21 Variants

IL-21 Variants with Substitutions at Position P79

Provided herein are polypeptides comprising an IL-21 variant with an amino acid residue substitution at position P79 corresponding to a wildtype human IL-21. In some embodiments, the wildtype human IL-21 comprises a sequence as set forth in SEQ ID NO: 1 or 2.

In some embodiments, the amino acid residue substitution at position P79 is P79E or P79C. In some specific embodiments, the amino acid residue substitution at position P79 is P79E. In other specific embodiments, the amino acid residue substitution at position P79 is P79C.

In some embodiments where the amino acid residue substitution at position P79 is P79C, the polypeptide further comprises one or more amino acid residue substitutions with cysteine in the region of positions 1-10 corresponding to the wildtype human IL-21. In some cases, the polypeptide further comprises Q1C. In some cases, the polypeptide further comprises G2C. In some cases, the polypeptide further comprises Q3C. In some cases, the polypeptide further comprises D4C. In some cases, the polypeptide further comprises RSC. In some cases, the polypeptide further comprises H6C. In some cases, the polypeptide further comprises M7C. In some cases, the polypeptide further comprises IBC. In some cases, the polypeptide further comprises R9C. In some cases, the polypeptide further comprises M10C. In some cases, the polypeptide further comprises a combination of two or more the above-described cysteine substitutions. In some cases, the one or more amino acid residue substitutions are selected from the group consisting of RSC, H6C and R9C.

In some cases, the cysteine residues of the one or more amino acid residue substitutions in the region of positions 1-10 form a disulfide linkage together with the cysteine residue at position P79. In some specific cases, the disulfide linkage is formed between the cysteine residue at position P79 and the cysteine residue at position 1. In some specific cases, the disulfide linkage is formed between the cysteine residue at position P79 and the cysteine residue at position 2. In some specific cases, the disulfide linkage is formed between the cysteine residue at position P79 and the cysteine residue at position 3. In some specific cases, the disulfide linkage is formed between the cysteine residue at position P79 and the cysteine residue at position 4. In some specific cases, the disulfide linkage is formed between the cysteine residue at position P79 and the cysteine residue at position 5. In some specific cases, the disulfide linkage is formed between the cysteine residue at position P79 and the cysteine residue at position 6. In some specific cases, the disulfide linkage is formed between the cysteine residue at position P79 and the cysteine residue at position 7. In some specific cases, the disulfide linkage is formed between the cysteine residue at position P79 and the cysteine residue at position 8. In some specific cases, the disulfide linkage is formed between the cysteine residue at position P79 and the cysteine residue at position 9. In some specific cases, the disulfide linkage is formed between the cysteine residue at position P79 and the cysteine residue at position 10.

In some embodiments where the polypeptide having an amino acid residue substitution at position P79 (e.g., P79E or P79C), the polypeptide further comprises one or more amino acid residue substitutions at positions selected from the group consisting of: R5, H6, R9, Q12, L13, D15, I16, S70, K72, K73, R76, and P78. In some specific embodiments where the polypeptide having an amino acid residue substitution at position P79 (e.g., P79E or P79C), the polypeptide further comprises one or more amino acid residue substitutions at positions selected from the group consisting of S70, K72, K73, and R76.

In specific embodiments, the polypeptide further comprises an amino acid residue substitution at position K73 corresponding to the wildtype human IL-21. In some cases, the amino acid residue substitution at position K73 is an aromatic amino acid. In specific cases, the amino acid residue substitution at position K73 is K73Y or K73F.

In specific embodiments, the polypeptide further comprises an amino acid residue substitution at position S70 corresponding to the wildtype human IL-21. In some cases, the amino acid residue substitution at S70 is S70L or S70A.

In specific embodiments, the polypeptide further comprises an amino acid residue substitution at position R76 corresponding to the wildtype human IL-21. In some cases, the amino acid residue substitution at R76 is R76F.

In specific embodiments, the polypeptide further comprises an amino acid residue substitution at position K72 corresponding to the wildtype human IL-21. In some cases, the amino acid residue substitution at K72 is a non-aromatic amino acid comprising a side chain hydroxyl. In some specific cases, the amino acid residue substitution at K72 is K72S.

In specific embodiments, the polypeptide further comprises an amino acid residue substitution at position Q12 corresponding to the wildtype human IL-21. In some cases, the amino acid residue substitution at Q12 is Q12W.

In specific embodiments, the polypeptide further comprises an amino acid residue substitution at position L13 corresponding to the wildtype human IL-21. In some cases, the amino acid residue substitution at L13 is L13A.

In specific embodiments, the polypeptide further comprises an amino acid residue substitution at position D15 corresponding to the wildtype human IL-21. In some cases, the amino acid residue substitution at D15 is D15L, D15K or D15R.

In specific embodiments, the polypeptide further comprises an amino acid residue substitution at position 116 corresponding to the wildtype human IL-21. In some cases, the amino acid residue substitution at 116 is 116R or 116W.

In specific embodiments, the polypeptide further comprises an amino acid residue substitution at position P78 corresponding to the wildtype human IL-21. In some cases, the amino acid residue substitution at P78 is P78G or P78E.

In some embodiments, the amino acid segment of STNAGRRQKHR (SEQ ID NO: 71) at positions 80-90 corresponding to the wildtype human IL-21 is substituted with a short peptide linker. In some embodiments, the length of the short peptide linker is at least 2, 3, 4, 5, 6, 7, 8, 9, or 10 amino acid residues. In some embodiments, the length of the short peptide linker is no greater than 2, 3, 4, 5, 6, 7, 8, 9, 10, 11, 12, 13, 14, 15, 16, 17, 18, 19, or 20 amino acid residues. In some embodiments, the length of the short peptide linker is from 4 to 10 amino acid residues. In some embodiments, the length of the short peptide linker is from 5 to 9 amino acid residues. In further embodiments, the short peptide linker comprises one or more Gly-Ser units. In further embodiments, the short peptide linker comprises one or more glutamic acid residues. In further embodiments, the short peptide linker comprises one glutamic acid residue. In specific embodiments where the short peptide linker comprises the glutamic acid residue, and the glutamic acid residue is in the middle of the short peptide linker. In some embodiments where the short peptide linker has a length of odd number (i.e., 2n+1), the glutamic acid residue is in the position n+1. In an exemplary embodiment where the short peptide linker is 5 amino-acid long, the glutamic acid residue is at the position of 3. In an exemplary embodiment where the short peptide linker is 7 amino-acid long, the glutamic acid residue is at the position of 4. In some embodiments where the short peptide linker has a length of odd number (i.e., 2n), the glutamic acid residue is in the position of either n or n+1. In an exemplary embodiment where the short peptide linker is 4 amino-acid long, the glutamic acid residue is at the position of 2 or 3. In an exemplary embodiment where the short peptide linker is 6 amino-acid long, the glutamic acid residue is at the position of 3 or 4. In specific embodiments, the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

In some embodiments, the polypeptide exhibits a lower binding affinity for IL-21 receptor (IL-21R) than the wildtype human IL-21. In specific embodiments, the polypeptide exhibits 2 to 1000 fold reduction in binding affinity for IL-21R as compared to the wildtype human IL-21. In specific embodiments, the polypeptide has a higher K_{D} value for IL-21R as compared to the wildtype human IL-21.

IL-21 Variants with Substitutions at Position 70

In another aspect, provided herein are polypeptides comprising an IL-21 variant with an amino acid residue substitution at position S70 corresponding to a wildtype human IL-21. In some embodiments, the wildtype human IL-21 comprises a sequence as set forth in SEQ ID NO: 1 or 2. In some cases, the amino acid residue substitution at position S70 is S70L or S70A.

In some embodiments, the polypeptide comprises an amino acid residue substitution at position S70 (e.g., S70L or S70A) and an amino acid residue substitution at position K73 corresponding to the wildtype human IL-21. In specific embodiments, the polypeptide comprises an amino acid residue substitution with an aromatic amino acid at position K73 corresponding to the wildtype human IL-21. In specific cases, the amino acid residue substitution at position K73 is K73Y or K73F. In other embodiments, the polypeptide further comprises one, two, or three, or more amino acid residue substitutions at positions selected from the group consisting of: R5, H6, R9, Q12, L13, D15, I16, K72, R76, P78 and P79. In specific embodiments, the polypeptide further comprises one, two, or three, or four amino acid residue substitutions at positions selected from the group consisting of: Q12, D15, I16, and K72. In some cases, the polypeptide comprises an amino acid residue substitution selected from the group consisting of: 116R and I16W. In other cases, the polypeptide comprises an amino acid residue substitution selected from the group consisting of: D15L, D15K, and D15R. In yet other cases, the polypeptide comprises an amino acid residue substitution selected from the group consisting of: K72S and Q12W.

In some embodiments, the polypeptide comprises an amino acid residue substitution at position S70 (e.g., S70L or S70A) and an amino acid residue substitution at position R76 corresponding to the wildtype human IL-21. In some cases, the amino acid residue substitution at position R76 is R76F. In some cases, the polypeptide further comprises one, two, or three, or more amino acid residue substitutions at positions selected from the group consisting of: R5, H6, R9, Q12, L13, D15, I16, K72, K73, P78 and P79. In some cases, the polypeptide further comprises one, two, or three, or four amino acid residue substitutions at positions selected from the group consisting of: Q12, D15, I16, and K72. In some cases, the polypeptide comprises an amino acid residue substitution selected from the group consisting of: 116R and 116W. In other cases, the polypeptide comprises an amino acid residue substitution selected from the group consisting of: D15L, D15K, and D15R. In yet other cases, the polypeptide comprises an amino acid residue substitution selected from the group consisting of K72S and Q12W.

In some embodiments, the amino acid segment of STNAGRRQKHR (SEQ ID NO: 71) at positions 80-90 corresponding to the wildtype human IL-21 is substituted with a short peptide linker. In some embodiments, the length of the short peptide linker is at least 2, 3, 4, 5, 6, 7, 8, 9, or 10 amino acid residues. In some embodiments, the length of the short peptide linker is no greater than 2, 3, 4, 5, 6, 7, 8, 9, 10, 11, 12, 13, 14, 15, 16, 17, 18, 19, or 20 amino acid residues. In some embodiments, the length of the short peptide linker is from 4 to 10 amino acid residues. In some embodiments, the length of the short peptide linker is from 5 to 9 amino acid residues. In further embodiments, the short peptide linker comprises one or more Gly-Ser units. In further embodiments, the short peptide linker comprises one or more glutamic acid residues. In further embodiments, the short peptide linker comprises one glutamic acid residue. In specific embodiments where the short peptide linker comprises the glutamic acid residue, and the glutamic acid residue is in the middle of the short peptide linker. In some embodiments where the short peptide linker has a length of odd number (i.e., 2n+1), the glutamic acid residue is in the position n+1. In an exemplary embodiment where the short peptide linker is 5 amino-acid long, the glutamic acid residue is at the position of 3. In an exemplary embodiment where the short peptide linker is 7 amino-acid long, the glutamic acid residue is at the position of 4. In some embodiments where the short peptide linker has a length of odd number (i.e., 2n), the glutamic acid residue is in the position of either n or n+1. In an exemplary embodiment where the short peptide linker is 4 amino-acid long, the glutamic acid residue is at the position of 2 or 3. In an exemplary embodiment where the short peptide linker is 6 amino-acid long, the glutamic acid residue is at the position of 3 or 4. In specific embodiments, the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

In some embodiments, the polypeptide exhibits a lower binding affinity for IL-21 receptor (IL-21R) than the wildtype human IL-21. In specific embodiments, the polypeptide exhibits 2 to 1000 fold reduction in binding affinity for IL-21R as compared to the wildtype human IL-21. In specific embodiments, the polypeptide has a higher K_{D} value for IL-21R as compared to the wildtype human IL-21.

IL-21 Variants with a Peptide Linker

In another aspect, provided herein are polypeptides comprising an interleukin-21 (IL-21) variant, wherein the amino acid segment of STNAGRRQKHR (SEQ ID NO: 71) at positions 80-90 corresponding to a wildtype human IL-21 is substituted with a short peptide linker. In some embodiments, the length of the short peptide linker is at least 2, 3, 4, 5, 6, 7, 8, 9, or 10 amino acid residues. In some embodiments, the length of the short peptide linker is no greater than 2, 3, 4, 5, 6, 7, 8, 9, 10, 11, 12, 13, 14, 15, 16, 17, 18, 19, or 20 amino acid residues. In some embodiments, the length of the short peptide linker is from 4 to 10 amino acid residues. In some embodiments, the length of the short peptide linker is from 5 to 9 amino acid residues. In some embodiments, the length of the short peptide linker is from 6 to 9 amino acid residues. In some embodiments, the length of the short peptide linker is from 7 to 9 amino acid residues. In further embodiments, the short peptide linker comprises one or more Gly-Ser units. In further embodiments, the short peptide linker comprises one or more glutamic acid residues. In further embodiments, the short peptide linker comprises one glutamic acid residue. In specific embodiments where the short peptide linker comprises the glutamic acid residue, and the glutamic acid residue is in the middle of the short peptide linker. In some embodiments where the short peptide linker has a length of odd number (i.e., 2n+1), the glutamic acid residue is in the position n+1. In an exemplary embodiment where the short peptide linker is 5 amino-acid long, the glutamic acid residue is at the position of 3. In an exemplary embodiment where the short peptide linker is 7 amino-acid long, the glutamic acid residue is at the position of 4. In some embodiments where the short peptide linker has a length of odd number (i.e., 2n), the glutamic acid residue is in the position of either n or n+1. In an exemplary embodiment where the short peptide linker is 4 amino-acid long, the glutamic acid residue is at the position of 2 or 3. In an exemplary embodiment where the short peptide linker is 6 amino-acid long, the glutamic acid residue is at the position of 3 or 4. In specific embodiments, the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

In some embodiments, the polypeptide comprises one or more amino acid residue substitutions at positions selected from the group consisting of: R5, H6, R9, Q12, L13, D15, I16, S70, K72, K73, R76, P78, and P79. In specific embodiments, the polypeptide comprises the amino acid residue substitution at position R5, and optionally the substitution is R5C. In specific embodiments, the polypeptide comprises the amino acid residue substitution at position H6, and optionally the substitution is H6C. In specific embodiments, the polypeptide comprises the amino acid residue substitution at position R9, and optionally the substitution is R9C. In specific embodiments, the polypeptide comprises the amino acid residue substitution at position Q12, and optionally the substitution is Q12W. In specific embodiments, the polypeptide comprises the amino acid residue substitution at position L13, and optionally the substitution is L13A. In specific embodiments, the polypeptide comprises the amino acid residue substitution at position D15, and optionally the substitution is D15L, D15K, or D15R. In specific embodiments, the polypeptide comprises the amino acid residue substitution at position 116, and optionally the substitution is 116R or 116W. In specific embodiments, the polypeptide comprises the amino acid residue substitution at position S70, and optionally the substitution is S70L or S70A. In specific embodiments, the polypeptide comprises the amino acid residue substitution at position K72, and optionally the substitution is K72S. In specific embodiments, the polypeptide comprises the amino acid residue substitution at position K73, and optionally the substitution is K73Y or K73F. In specific embodiments, the polypeptide comprises the amino acid residue substitution at position R76, and optionally the substitution is R76F. In specific embodiments, the polypeptide comprises the amino acid residue substitution at position P78, and optionally the substitution is P78G or P78E. In specific embodiments, the polypeptide comprises the amino acid residue substitution at position P79, and optionally the substitution is P79E or P79C.

In some embodiments, the polypeptide exhibits an improved thermostability as compared to the wildtype human IL-21. In some embodiments, the polypeptide exhibits a lower binding affinity for IL-21 receptor (IL-21R) than wild-type IL-21. In some embodiments, the polypeptide exhibits 2 to 1000 fold reduction in binding affinity for IL-21R as compared to wild-type IL-21. In some embodiments, the polypeptide has a higher K_{D} value for IL-21R as compared to wild-type IL-21.

IL-21 Variants with One or More Specific Substitutions

In another aspect, provided herein are polypeptides comprising an interleukin-21 (IL-21) variant comprising a wildtype human IL-21 amino acid sequence with at least one mutation selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C. In some embodiments, the wildtype human IL-21 comprises a sequence as set forth in SEQ ID NO: 1 or 2.

In some embodiments, the polypeptide described herein comprises only one mutation selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises two mutations, the each of the two mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises three mutations, the each of the three mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises four mutations, the each of the four mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises five mutations, the each of the five mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises six mutations, the each of the six mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises seven mutations, the each of the two mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises eight mutations, the each of the eight mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises nine mutations, the each of the nine mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises ten mutations, the each of the ten mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises eleven mutations, the each of the eleven mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises twelve mutations, the each of the twelve mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the polypeptide described herein comprises thirteen mutations, the each of the thirteen mutations is selected from R5C, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

In some embodiments, the amino acid segment of STNAGRRQKHR (SEQ ID NO: 71) at positions 80-90 corresponding to the wildtype human IL-21 is substituted with a short peptide linker. In some embodiments, the length of the short peptide linker is at least 2, 3, 4, 5, 6, 7, 8, 9, or 10 amino acid residues. In some embodiments, the length of the short peptide linker is no greater than 2, 3, 4, 5, 6, 7, 8, 9, 10, 11, 12, 13, 14, 15, 16, 17, 18, 19, or 20 amino acid residues. In some embodiments, the length of the short peptide linker is from 4 to 10 amino acid residues. In some embodiments, the length of the short peptide linker is from 5 to 9 amino acid residues. In some embodiments, the length of the short peptide linker is from 7 to 9 amino acid residues. In further embodiments, the short peptide linker comprises one or more Gly-Ser units. In further embodiments, the short peptide linker comprises one or more glutamic acid residues. In further embodiments, the short peptide linker comprises one glutamic acid residue. In specific embodiments where the short peptide linker comprises the glutamic acid residue, and the glutamic acid residue is in the middle of the short peptide linker. In some embodiments where the short peptide linker has a length of odd number (i.e., 2n+1), the glutamic acid residue is in the position n+1. In an exemplary embodiment where the short peptide linker is 5 amino-acid long, the glutamic acid residue is at the position of 3. In an exemplary embodiment where the short peptide linker is 7 amino-acid long, the glutamic acid residue is at the position of 4. In some embodiments where the short peptide linker has a length of odd number (i.e., 2n), the glutamic acid residue is in the position of either n or n+1. In an exemplary embodiment where the short peptide linker is 4 amino-acid long, the glutamic acid residue is at the position of 2 or 3. In an exemplary embodiment where the short peptide linker is 6 amino-acid long, the glutamic acid residue is at the position of 3 or 4. In specific embodiments, the short peptide linker is selected from the group consisting of GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

In another aspect, provided herein is a polypeptide comprising an IL-21 variant, wherein the polypeptide comprises amino acid residue mutation P79E. In another aspect, provided herein is a polypeptide comprising an IL-21 variant, wherein the polypeptide comprises amino acid residue mutations P79E K73Y, and P79E. In another aspect, provided herein is a polypeptide comprising an IL-21 variant, wherein the polypeptide comprises amino acid residue mutations K73F and P79E. In another aspect, provided herein is a polypeptide comprising an IL-21 variant, wherein the polypeptide comprises amino acid residue mutations S70L, K73F, and P79E. In another aspect, provided herein is a polypeptide comprising an IL-21 variant, wherein the polypeptide comprises amino acid residue mutations S70L, K73Y, and P79E. In another aspect, provided herein is a polypeptide comprising an IL-21 variant, wherein the polypeptide comprises amino acid residue mutations S70L, K72S, K73F, and P79E. In another aspect, provided herein is a polypeptide comprising an IL-21 variant, wherein the polypeptide comprises amino acid residue mutations S70L, K72S, K73Y, and P79E. In another aspect, provided herein is a polypeptide comprising an IL-21 variant, wherein the polypeptide comprises amino acid residue mutations D15K, S70L, K73F, and P79E. In another aspect, provided herein is a polypeptide comprising an IL-21 variant, wherein the polypeptide comprises amino acid residue mutations D15R, S70L, K73F, and P79E. In another aspect, provided herein is a polypeptide comprising an IL-21 variant, wherein the polypeptide comprises amino acid residue mutations 116W, S70L, K73F, and P79E. In another aspect, provided herein is a polypeptide comprising an IL-21 variant, wherein the polypeptide comprises amino acid residue mutations D15L, S70L, K73F, and P79E. In another aspect, provided herein is a polypeptide comprising an IL-21 variant, wherein the polypeptide comprises amino acid residue mutations L13A, S70L, K73F, and P79E. In another aspect, provided herein is a polypeptide comprising an IL-21 variant, wherein the polypeptide comprises amino acid residue mutations D15R, R76F, P78E, and P79E. In another aspect, provided herein is a polypeptide comprising an IL-21 variant, wherein the polypeptide comprises amino acid residue mutations D15L, 116R, S70L, K73Y, and P79E. In another aspect, provided herein is a polypeptide comprising an IL-21 variant, wherein the polypeptide comprises amino acid residue mutations D15K, 116W, S70L, R76F, and P79E. In another aspect, provided herein is a polypeptide comprising an IL-21 variant, wherein the polypeptide comprises amino acid residue mutations D15R, I16W, S70L, R76F, and P79E. In another aspect, provided herein is a polypeptide comprising an IL-21 variant, wherein the polypeptide comprises amino acid residue mutations STNAGRRQKHR (SEQ ID NO: 71) substituted with GGGSEGGGS (SEQ ID NO: 72) (STNAGRRQKHR (SEQ ID NO: 71)/GGGSEGGGS (SEQ ID NO: 72)). In another aspect, provided herein is a polypeptide comprising an IL-21 variant, wherein the polypeptide comprises amino acid residue mutations P79E and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations K73Y, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations K73F, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations S70L, K73F, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations S70L, K73Y, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations D15K, S70L, K73F, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations D15R, S70L, K73F, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations 116W, S70L, K73F, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations S70L, K72S, K73F, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations S70L, K72S, K73Y, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations D15R, I16W, S70L, K73Y, P79E, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations D15L, 116R, S70L, K73Y, P79E, and STNAGRRQKHR (SEQ ID NO: 71)/GGGSEGGGS (SEQ ID NO: 72).

In another aspect, provided herein is a polypeptide comprising an IL-21 variant, wherein the polypeptide comprises amino acid residue mutation P79C. In another aspect, provided herein is a polypeptide comprising an IL-21 variant, wherein the polypeptide comprises amino acid residue mutations R5C, P79C. In another aspect, provided herein is a polypeptide comprising an IL-21 variant, wherein the polypeptide comprises amino acid residue mutations H6C and P79C. In another aspect, provided herein is a polypeptide comprising an IL-21 variant, wherein the polypeptide comprises amino acid residue mutations R9C and P79C. In another aspect, provided herein is a polypeptide comprising an IL-21 variant, wherein the polypeptide comprises amino acid residue mutations R5C, R76F, and P79C. In another aspect, provided herein is a polypeptide comprising an IL-21 variant, wherein the polypeptide comprises amino acid residue mutations H6C, R76F, and P79C. In another aspect, provided herein is a polypeptide comprising an IL-21 variant, wherein the polypeptide comprises amino acid residue mutations H6C, K73Y, and P79C. In another aspect, provided herein is a polypeptide comprising an IL-21 variant, wherein the polypeptide comprises amino acid residue mutations H6C, K73F, and P79C. In another aspect, provided herein is a polypeptide comprising an IL-21 variant, wherein the polypeptide comprises amino acid residue mutations H6C, S70L, K73Y, and P79C. In another aspect, provided herein is a polypeptide comprising an IL-21 variant, wherein the polypeptide comprises amino acid residue mutations H6C, S70L, K73F, P79C; R9C, D15R, P78G, and P79C. In another aspect, provided herein is a polypeptide comprising an IL-21 variant, wherein the polypeptide comprises amino acid residue mutations H6C, D15L, I16R, S70L, K73Y, and P79C. In another aspect, provided herein is a polypeptide comprising an IL-21 variant, wherein the polypeptide comprises amino acid residue mutations R9C, D15L, I16R, S70L, K73Y, and P79C. In another aspect, provided herein is a polypeptide comprising an IL-21 variant, wherein the polypeptide comprises amino acid residue mutations H6C, P79C, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations H6C, K73Y, P79C, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations H6C, K73F, P79C, and STNAGRRQKHR (SEQ ID NO: 71)/GGGSEGGGS (SEQ ID NO: 72). In another aspect, provided herein is a polypeptide comprising an IL-21 variant, wherein the polypeptide comprises amino acid residue mutations H6C, S70L, P79C, and STNAGRRQKHR (SEQ ID NO: 71)/GGGSEGGGS (SEQ ID NO: 72).

In another aspect, provided herein is a polypeptide comprising an IL-21 variant, wherein the polypeptide comprises amino acid residue mutation K73Y. In another aspect, provided herein is a polypeptide comprising an IL-21 variant, wherein the polypeptide comprises amino acid residue mutation K73F. In another aspect, provided herein is a polypeptide comprising an IL-21 variant, wherein the polypeptide comprises amino acid residue mutation S70L. In another aspect, provided herein is a polypeptide comprising an IL-21 variant, wherein the polypeptide comprises amino acid residue mutation S70A. In another aspect, provided herein is a polypeptide comprising an IL-21 variant, wherein the polypeptide comprises amino acid residue mutations S70L and K73Y. In another aspect, provided herein is a polypeptide comprising an IL-21 variant, wherein the polypeptide comprises amino acid residue mutations S70L and K73F. In another aspect, provided herein is a polypeptide comprising an IL-21 variant, wherein the polypeptide comprises amino acid residue mutations S70L, K72S, and K73Y. In another aspect, provided herein is a polypeptide comprising an IL-21 variant, wherein the polypeptide comprises amino acid residue mutations S70A, K72S, and K73F. In another aspect, provided herein is a polypeptide comprising an IL-21 variant, wherein the polypeptide comprises amino acid residue mutations Q12W, S70A, and R76F. In another aspect, provided herein is a polypeptide comprising an IL-21 variant, wherein the polypeptide comprises amino acid residue mutations D15K, S70A, and R76F. In another aspect, provided herein is a polypeptide comprising an IL-21 variant, wherein the polypeptide comprises amino acid residue mutations D15L, S70A, and R76F. In another aspect, provided herein is a polypeptide comprising an IL-21 variant, wherein the polypeptide comprises amino acid residue mutations D15R, S70A, and R76F. In another aspect, provided herein is a polypeptide comprising an IL-21 variant, wherein the polypeptide comprises amino acid residue mutations 116R, S70L, and K73Y. In another aspect, provided herein is a polypeptide comprising an IL-21 variant, wherein the polypeptide comprises amino acid residue mutations D15L, I16R, S70L, and K73Y. In another aspect, provided herein is a polypeptide comprising an IL-21 variant, wherein the polypeptide comprises amino acid residue mutations D15K, I16W, S70L, and K73Y. In another aspect, provided herein is a polypeptide comprising an IL-21 variant, wherein the polypeptide comprises amino acid residue mutations DiSK, I16W, S70L, and R76F. In another aspect, provided herein is a polypeptide comprising an IL-21 variant, wherein the polypeptide comprises amino acid residue mutations D15R, I16W, S70L, and R76F. In another aspect, provided herein is a polypeptide comprising an IL-21 variant, wherein the polypeptide comprises amino acid residue mutations I16W, S70L, K72S, and K73F. In another aspect, provided herein is a polypeptide comprising an IL-21 variant, wherein the polypeptide comprises amino acid residue mutations D15R, I16W, S70L, and K73Y. In another aspect, provided herein is a polypeptide comprising an IL-21 variant, wherein the polypeptide comprises amino acid residue mutations D15K, 116W, S70L, K72S, and K73Y.

In another aspect, provided herein is a polypeptide comprising IL-21 variant, wherein the polypeptide comprises a sequence as set forth in any one of SEQ ID Nos. 5-62. In another aspect, provided herein is a polypeptide comprising IL-21 variant, wherein the polypeptide consists of a sequence as set forth in any one of SEQ ID Nos. 5-62. In some embodiment, the polypeptide comprises or consists of a sequence as set forth in SEQ ID No. 5. In some embodiment, the polypeptide comprises or consists of a sequence as set forth in SEQ ID No. 6. In some embodiment, the polypeptide comprises or consists of a sequence as set forth in SEQ ID No. 7. In some embodiment, the polypeptide comprises or consists of a sequence as set forth in SEQ ID No. 8. In some embodiment, the polypeptide comprises or consists of a sequence as set forth in SEQ ID No. 9. In some embodiment, the polypeptide comprises or consists of a sequence as set forth in SEQ ID No. 10. In some embodiment, the polypeptide comprises or consists of a sequence as set forth in SEQ ID No. 11. In some embodiment, the polypeptide comprises or consists of a sequence as set forth in SEQ ID No. 12. In some embodiment, the polypeptide comprises or consists of a sequence as set forth in SEQ ID No. 13. In some embodiment, the polypeptide comprises or consists of a sequence as set forth in SEQ ID No. 14. In some embodiment, the polypeptide comprises or consists of a sequence as set forth in SEQ ID No. 15. In some embodiment, the polypeptide comprises or consists of a sequence as set forth in SEQ ID No. 16. In some embodiment, the polypeptide comprises or consists of a sequence as set forth in SEQ ID No. 17. In some embodiment, the polypeptide comprises or consists of a sequence as set forth in SEQ ID No.18. In some embodiment, the polypeptide comprises or consists of a sequence as set forth in SEQ ID No.19. In some embodiment, the polypeptide comprises or consists of a sequence as set forth in SEQ ID No. 20. In some embodiment, the polypeptide comprises or consists of a sequence as set forth in SEQ ID No. 21. In some embodiment, the polypeptide comprises or consists of a sequence as set forth in SEQ ID No. 22. In some embodiment, the polypeptide comprises or consists of a sequence as set forth in SEQ ID No.23. In some embodiment, the polypeptide comprises or consists of a sequence as set forth in SEQ ID No.24. In some embodiment, the polypeptide comprises or consists of a sequence as set forth in SEQ ID No.25. In some embodiment, the polypeptide comprises or consists of a sequence as set forth in SEQ ID No.26. In some embodiment, the polypeptide comprises or consists of a sequence as set forth in SEQ ID No.27. In some embodiment, the polypeptide comprises or consists of a sequence as set forth in SEQ ID No.28. In some embodiment, the polypeptide comprises or consists of a sequence as set forth in SEQ ID No.29. In some embodiment, the polypeptide comprises or consists of a sequence as set forth in SEQ ID No. 30. In some embodiment, the polypeptide comprises or consists of a sequence as set forth in SEQ ID No. 31. In some embodiment, the polypeptide comprises or consists of a sequence as set forth in SEQ ID No.32. In some embodiment, the polypeptide comprises or consists of a sequence as set forth in SEQ ID No.33. In some embodiment, the polypeptide comprises or consists of a sequence as set forth in SEQ ID No.34. In some embodiment, the polypeptide comprises or consists of a sequence as set forth in SEQ ID No.35. In some embodiment, the polypeptide comprises or consists of a sequence as set forth in SEQ ID No.36. In some embodiment, the polypeptide comprises or consists of a sequence as set forth in SEQ ID No.37. In some embodiment, the polypeptide comprises or consists of a sequence as set forth in SEQ ID No.38. In some embodiment, the polypeptide comprises or consists of a sequence as set forth in SEQ ID No.39. In some embodiment, the polypeptide comprises or consists of a sequence as set forth in SEQ ID No.40. In some embodiment, the polypeptide comprises or consists of a sequence as set forth in SEQ ID No.41. In some embodiment, the polypeptide comprises or consists of a sequence as set forth in SEQ ID No.42. In some embodiment, the polypeptide comprises or consists of a sequence as set forth in SEQ ID No.43. In some embodiment, the polypeptide comprises or consists of a sequence as set forth in SEQ ID No.44. In some embodiment, the polypeptide comprises or consists of a sequence as set forth in SEQ ID No.45. In some embodiment, the polypeptide comprises or consists of a sequence as set forth in SEQ ID No.46. In some embodiment, the polypeptide comprises or consists of a sequence as set forth in SEQ ID No. 47. In some embodiment, the polypeptide comprises or consists of a sequence as set forth in SEQ ID No.48. In some embodiment, the polypeptide comprises or consists of a sequence as set forth in SEQ ID No.49. In some embodiment, the polypeptide comprises or consists of a sequence as set forth in SEQ ID No.50. In some embodiment, the polypeptide comprises or consists of a sequence as set forth in SEQ ID No.51. In some embodiment, the polypeptide comprises or consists of a sequence as set forth in SEQ ID No.52. In some embodiment, the polypeptide comprises or consists of a sequence as set forth in SEQ ID No.53. In some embodiment, the polypeptide comprises or consists of a sequence as set forth in SEQ ID No.54. In some embodiment, the polypeptide comprises or consists of a sequence as set forth in SEQ ID No.55. In some embodiment, the polypeptide comprises or consists of a sequence as set forth in SEQ ID No.56. In some embodiment, the polypeptide comprises or consists of a sequence as set forth in SEQ ID No.57. In some embodiment, the polypeptide comprises or consists of a sequence as set forth in SEQ ID No.58. In some embodiment, the polypeptide comprises or consists of a sequence as set forth in SEQ ID No.59. In some embodiment, the polypeptide comprises or consists of a sequence as set forth in SEQ ID No. 60. In some embodiment, the polypeptide comprises or consists of a sequence as set forth in SEQ ID No. 61. In some embodiment, the polypeptide comprises or consists of a sequence as set forth in SEQ ID No. 62.

In some embodiments, the polypeptide exhibits a lower binding affinity for IL-21 receptor (IL-21R) than the wildtype human IL-21. In some embodiments, the polypeptide exhibits 2 to 1000 fold reduction in binding affinity for IL-21R as compared to the wildtype human IL-21. In some embodiments, the polypeptide has a higher K_{D} value for IL-21R as compared to the wildtype human IL-21.

In some embodiments, the wildtype human IL-21 comprises the amino acid sequence of SEQ ID NO: 1 or 2. In some embodiments, the wildtype human IL-21 consists of the amino acid sequence of SEQ ID NO: 1 or 2. In some embodiments, the IL-21 variant comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 1 or 2. In some embodiments, an IL-21 polypeptide comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 5-62.

In some embodiments, an amino acid residue of the IL-21 variant described herein is mutated to an unnatural amino acid. In some embodiments, the polypeptide described herein comprises an unnatural amino acid, wherein the cytokine is conjugated to the protein, wherein the point of attachment is not the unnatural amino acid. In some embodiments, the polypeptide described herein comprises an unnatural amino acid, wherein the cytokine is conjugated to the protein, wherein the point of attachment is the unnatural amino acid. In some embodiments, an amino acid residue of the IL-21 variant described herein is mutated prior to binding to (or reacting with) an additional moiety. In some embodiments, mutation to an unnatural amino acid reduces the likelihood of or minimizes a self-antigen response of the immune system.

Non-limiting examples of unnatural amino acid include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, azido-lysine (AzK), an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy amino acid; an amino thioacid; an α,α-disubstituted amino acid; a D-amino acid; a cyclic amino acid other than proline or histidine, an aromatic amino acid other than phenylalanine, tyrosine or tryptophan, N6-azidoethoxy-L-lysine (AzK), N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, and selenocysteine.

In some embodiments, the unnatural amino acid comprises p-acetyl-L-phenylalanine, p-azidomethyl-L-phenylalanine (pAMF), p-iodo-L-phenylalanine, O-methyl-L-tyrosine, p-methoxyphenylalanine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, L-phosphoserine, phosphonoserine, phosphonotyrosine, p-bromophenylalanine, p-amino-L-phenylalanine, and/or isopropyl-L-phenylal anine. In some embodiments, the unnatural amino acid is 3-aminotyrosine, 3-nitrotyrosine, 3,4-dihydroxy-phenylalanine, or 3-iodotyrosine. In some embodiments, the unnatural amino acid is phenylselenocysteine. In some embodiments, the unnatural amino acid is a benzophenone, ketone, iodide, methoxy, acetyl, benzoyl, or azide containing phenylalanine derivative. In some embodiments, the unnatural amino acid is a benzophenone, ketone, iodide, methoxy, acetyl, benzoyl, or azide-containing lysine derivative.

In some embodiments, the unnatural amino acid comprises a selective reactive group, or a reactive group for site-selective labeling of a target polypeptide. In some embodiments, the chemistry is a biorthogonal reaction (e.g., biocompatible and selective reactions). In some cases, the chemistry is a Cu(I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling. In some embodiments, the unnatural amino acid comprises a photoreactive group, which crosslinks, upon irradiation with, e.g., UV. In some embodiments, the unnatural amino acid comprises a photo-caged amino acid.

In some embodiments, the unnatural amino acid is a para-substituted, meta-substituted, or an ortho-substituted amino acid derivative.

### IL-21 Binding and Activity

In some embodiments, the polypeptides disclosed herein exhibits a decreased affinity to the IL-21R relative to the wildtype human IL-21. In some embodiments, the decreased affinity is about 10%, about 20%, about 30%, about 40%, about 50%, about 60%, about 70%, about 80%, about 90%, about 95%, about 99%, or greater decrease in binding affinity to IL-21R relative to the wildtype human IL-21.

Binding affinity of the polypeptides provided herein to a target receptor or a subunit thereof can be assessed by measuring the dissociation constant (K_{D}). In some embodiments, the polypeptides provided herein binds to IL-21R with a K_{D} that is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1000-fold higher than the K_{D} of the wildtype IL-21. In some embodiments, the polypeptides provided herein binds to IL-21R with a K_{D} that is about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold, about 700-fold, about 800-fold, about 900-fold, or about 1000-fold higher than the K_{D} of the wildtype IL-21.

In some embodiments, the polypeptide provided herein binds to IL-21R with a K_{D} of at least 10⁻¹⁰ M, at least 10⁻⁹ M, at least 10⁻⁸ M, at least 10⁻⁷ M, at least 10⁻⁶ M, at least 10⁻⁵ M, at least 10⁻⁴ M, at least 10⁻³ M, at least 10⁻² M, at least 10⁻¹ M, or greater. In some embodiments, the polypeptide provided herein binds to IL-21R with a K_{D} of about 10⁻¹⁰ M, about 10⁻⁹ M, about 10⁻⁸ M, about 10⁻⁷ M, about 10⁻⁶ M, about 10⁻⁵ M, about 10⁻⁴ M, about 10⁻³ M, about 10⁻² M, or about 10⁻¹ M. In some embodiments, the polypeptide provided herein binds to IL-21R with a K_{D} of 10⁻¹⁰ M to 10⁻¹ M, 10⁻¹⁰ M to 10⁻² M, 10⁻¹⁰ M to 10⁻³ M, 10⁻¹⁰ M to 10⁻⁴ M, 10⁻¹⁰ M to 10⁻⁵ M, 10⁻¹⁰ M to 10⁻⁶ M, 10⁻¹⁰ M to 10⁻⁷ M, 10⁻¹⁰ M to 10⁻¹ M, 10⁻¹⁰ M to 10⁻⁹ M, 10⁻⁹ M to 10⁻¹ M, 10⁻⁹ M to 10⁻² M, 10⁻⁹ M to 10⁻³ M, 10⁻⁹ M to 10⁻⁴ M, 10⁻⁹ M to 10⁻⁵ M, 10⁻⁹ M to 10⁻⁶ M, 10⁻⁹ M to 10⁻⁷ M, 10⁻⁹ M to 10⁻⁸ M, 10⁻⁸ M to 10⁻¹ M, 10⁻⁸ M to 10⁻² M, 10⁻⁸ M to 10⁻³ M, 10⁻⁸ M to 10⁻⁴ M, 10⁻¹ M to 10⁻¹ M, 10⁻¹ M to 10⁻⁶ M, 10⁻¹ M to 10⁻⁷ M, 10⁻⁷ M to 10⁻¹ M, 10-?M to 10⁻² M, 10⁻⁷ M to 10⁻³ M, 10⁻⁷ M to 10⁻⁴ M, 10⁻⁷ M to 10⁻⁵ M, 10⁻⁷ M to 10⁻⁶ M, 10⁻⁶ M to 10⁻¹ M, 10⁻⁶ M to 10⁻² M, 10⁻⁶ M to 10⁻³ M, 10⁻⁶ M to 10⁻⁴ M, or 10⁻⁶ M to 10⁻⁵ M.

IL-21 signaling activity by the IL-21 variant provided herein can be assessed by IL21-mediated phosphorylation of STAT3. In some embodiments, HuT78 cells can be treated with the IL-21 polypeptides provided herein and phosphorylation of STAT3 can be subsequently analyzed. STAT3 phosphorylation can be used to calculate the half-maximal effect concentration or EC₅₀. Relative activity of the IL-21 variant provided herein can be determined by comparing EC₅₀ values.

In some embodiments, the IL-21 variant provided herein has a relative activity that is at least 1-fold, at least 1.5-fold, 2-fold, at least 2.5-fold, at least 3-fold, at least 3.5-fold, at least 4-fold, at least 4.5-fold, at least 5-fold, at least 5.5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold lower than the STAT3 phosphorylation activity of the wildtype IL-2. In some embodiments, the IL-21 variant provided herein has an EC₅₀ of STAT3 phosphorylation activity that is at least 1-fold, at least 1.5-fold, 2-fold, at least 2.5-fold, at least 3-fold, at least 3.5-fold, at least 4-fold, at least 4.5-fold, at least 5 -fold, at least 5.5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold higher than the one of the wildtype IL-2.

### Conjugates

In another aspect, provided herein are conjugates comprising the polypeptide disclosed herein and at least one additional moiety. In other embodiments, the at least one additional moiety comprises a protein or a binding fragment thereof. In additional embodiments, the at least one additional moiety comprises a peptide. In additional embodiments, the at least one additional moiety comprises a nucleic acid. In additional embodiments, the at least one additional moiety comprises a small molecule.

In some embodiments, the at least one additional moiety is attached to an unnatural or natural amino acid in the polypeptides. In some embodiments, the polypeptide comprises the at least one additional moiety attached to a natural amino acid in the polypeptide. In some embodiments, the polypeptide comprises the at least one additional moiety attached to an unnatural amino acid in the polypeptide. In some embodiments, the at least one additional moiety is attached to the N- or C-terminus amino acid of the polypeptide. In some embodiments, various combinations sites are disclosed herein, for example, a first additional moiety is attached to an amino acid in the polypeptide, and a second additional moiety is attached to the N- or C-terminus of the polypeptide. In some embodiments, a single additional moiety is attached to multiple residues of the polypeptide (e.g., a staple). In some embodiments, the at least one additional moiety is attached to both the N- and C-terminus amino acids of the polypeptide.

In some embodiments, the at least one additional moiety can be linked to an N-terminus of an IL-21 variant moiety. In some embodiments, the at least one additional moiety can be linked to a C-terminus of an IL-21 variant moiety.

### Water-Soluble Polymers

In some embodiments, the at least one additional moiety is a water-soluble polymer. In some embodiments, the water-soluble polymer is a non-peptidic, non-toxic, and biocompatible. A substance can be considered biocompatible if the beneficial effects associated with use of the substance alone or with another substance (e.g., an active agent such as an IL-21 or the polypeptide disclosed herein) in connection with living tissues (e.g., administration to a subject) outweighs any deleterious effects as evaluated by a clinician, e.g., a physician, a toxicologist, or a clinical development specialist.

In some embodiments, a water-soluble polymer is non-immunogenic. A substance can be considered non-immunogenic if the intended use of the substance in vivo does not produce an undesired immune response (e.g., the formation of antibodies), or if an immune response is produced, that such a response is not deemed clinically significant or important as evaluated by a clinician, e.g., a physician, a toxicologist, or a clinical development specialist.

In some instances, the water-soluble polymer is characterized as having from about 2 to about 300 termini. Non-limiting examples of water-soluble polymers include poly(alkylene glycols), such as polyethylene glycol (PEG), poly(propylene glycol) (PPG), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethaerylate), poly(saccharides), poly(a-hydroxy acid), poly(vinyl alcohol) (PVA), polyacrylamide (PAAm), poly(N-(2-hydroxypropyl) methacrylamide) (PHPMA), polydimethylacrylamide (PDAAm), polyphosphazene, polyoxazolines (POZ), poly(N-acryloylmorpholine), and any combination thereof.

In some embodiments, the water-soluble polymer is not limited to a particular structure. In some cases, the water-soluble polymer is linear (e.g., an end capped, e.g., an alkoxy PEG or a bifunctional PEG), branched or multi-armed (e.g., forked PEG or PEG attached to a polyol core), a dendritic (or star) architecture, each with or without one or more degradable linkages. Moreover, the internal structure of the water-soluble polymer can be organized in any number of different repeat patterns, such as homopolymer, alternating copolymer, random copolymer, block copolymer, alternating tripolymer, random tripolymer, or block tripolymer.

In some embodiments, the weight-average molecular weight of a water-soluble polymer is from about 100 Daltons (Da) to about 150,000 Da. Non-limiting examples of weight-average molecular weight ranges include from about 5,000 Da to about 100,000 Da, from about 6,000 Da to about 90,000 Da, from about 10,000 Da to about 85,000 Da, from about 10,000 Da to about 85,000 Da, from about 20,000 Da to about 85,000 Da, from about 53,000 Da to about 85,000 Da, from about 25,000 Da to about 120,000 Da, from about 29,000 Da to about 120,000 Da, from about 35,000 Da to about 120,000 Da, and from about 40,000 Da to about 120,000 Da.

Non-limiting examples of weight-average molecular weights for a water-soluble polymer include about 100 Da, about 200 Da, about 300 Da, about 400 Da, about 500 Da, about 600 Da, about 700 Da, about 750 Da, about 800 Da, about 900 Da, about 1,000 Da, about 1,500 Da, about 2,000 Da, about 2,200 Da, about 2,500 Da, about 3,000 Da, about 4,000 Da, about 4,400 Da, about 4,500 Da, about 5,000 Da, about 5,500 Da, about 6,000 Da, about 7,000 Da, about 7,500 Da, about 8,000 Da, about 9,000 Da, about 10,000 Da, about 11,000 Da, about 12,000 Da, about 13,000 Da, about 14,000 Da, about 15,000 Da, about 20,000 Da, about 22,500 Da, about 25,000 Da, about 30,000 Da, about 35,000 Da, about 40,000 Da, about 45,000 Da, about 50,000 Da, about 55,000 Da, about 60,000 Da, about 65,000 Da, about 70,000 Da, and about 75,000 Da. Branched versions of the water-soluble polymer (e.g., a branched 40,000 Da water-soluble polymer comprised of two 20,000 Da polymers) having a total molecular weight of any of the foregoing can also be used. In some embodiments, the conjugate does not have any PEG moieties attached, either directly or indirectly, with a PEG having a weight average molecular weight of less than about 6,000 Da.

PEGs can comprise a number of (OCH2CH2) monomers or (CH2CH2O) monomers. The number of repeating units can be identified by the subscript “n” in “(OCH2CH2)n.” Thus, the value of “n” typically can fall within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, the number of repeating units (i.e., “n”) can be determined by dividing the total weight-average molecular weight of the polymer by the molecular weight of the repeating monomer.

In some instances, the water-soluble polymer is an end-capped polymer, i.e., having at least one terminus capped with a relatively inert group, such as a lower C1-C6 alkoxy group, or a hydroxyl group. For example, the water-soluble polymer can be a methoxy-PEG (mPEG), which is a linear form of PEG in which one terminus of the PEG is a methoxy (—OCH3) group, while the other terminus is a hydroxyl or other functional group that can be optionally chemically modified. Non-limiting examples of water-soluble polymers include linear or branched discrete PEG (dPEG); linear, branched, or forked PEGs; and Y-shaped PEG derivatives.

In some embodiments, the polypeptide described herein is conjugated to a water-soluble polymer selected from poly(alkylene glycols), such as polyethylene glycol (PEG), poly(propylene glycol) (PPG), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol) (PVA), polyacrylamide (PAAm), poly(N-(2-hydroxypropyl) methacrylamide) (PHPMA), polydimethylacrylamide (PDAAm), polyphosphazene, polyoxazolines (POZ), poly(N-acryloylmorpholine), and a combination thereof. For example, an IL-21 polypeptide is conjugated to PEG (e.g., PEGylated).

In some embodiments, the water-soluble polymer comprises a polyglycerol (PG), e.g., a HPG, a LPG, a midfunctional PG, a linear-block-hyperbranched PG, or a side-chain functional PG. In some cases, the polyglycerol is a hyperbranched PG (HPG).

In some embodiments, a water-soluble polymer is a degradable synthetic PEG alternative. Non-limiting examples of degradable synthetic PEG alternatives include poly[oligo(ethylene glycol)methyl methacrylate](POEGMA); backbone modified PEG derivatives generated by polymerization of telechelic, or di-end-functionalized PEG-based macromonomers; PEG derivatives comprising comonomers comprising degradable linkage such as poly[(ethylene oxide)-co-(methylene ethylene oxide)][P(EO-co-MEO)], cyclic ketene acetals such as 5,6-benzo-2-methylene-1,3-dioxepane (BMDO), 2-methylene-1,3-dioxepane (MDO), and 2-methylene-4-phenyl-1,3-dioxolane (MPDL) copolymerized with OEGMA, or poly-(s-caprolactone)-graft-poly(ethylene oxide) (PCL-g-PEO).

In some embodiments, the water-soluble polymer comprises a poly(zwitterions). Non-limiting examples of poly(zwitterions) include poly(sulfobetaine methacrylate) (PSBMA), poly(carboxybetaine methacrylate) (PCBMA), and poly(2-methyacryloyloxyethyl phosphorylcholine) (PMPC).

In some embodiments, the water-soluble polymer comprises a polycarbonate. Non-limiting examples of polycarbonates include pentafluorophenyl 5-methyl-2-oxo-1,3-dioxane-5-carboxylate (MTC-OC6F5).

In some embodiments, the water-soluble polymer comprises a polymer hybrid, such as for example, a polycarbonate/PEG polymer hybrid, a peptide/protein-polymer conjugate, or a hydroxylcontaining and/or zwitterionic derivatized polymer (e.g., a hydroxylcontaining and/or zwitterionic derivatized PEG polymer).

In some embodiments, the water-soluble polymer comprises a polysaccharide. Non-limiting examples of polysaccharides include dextran, polysialic acid (PSA), hyaluronic acid (HA), amylose, heparin, heparan sulfate (HS), dextrin, or hydroxyethyl starch (HES).

In some embodiments, the water-soluble polymer comprises a glycan. Non-limiting examples of glycans include N-linked glycans, O-linked glycans, glycolipids, O-GlcNAc, and glycosaminoglycans.

In some embodiments, the water-soluble polymer comprises a polyoxazoline polymer. A polyoxazoline polymer is a linear synthetic polymer, and similar to PEG, comprises a low polydispersity. In some instances, a polyoxazoline polymer is a polydispersed polyoxazoline polymer, characterized with an average molecule weight. In some cases, the average molecule weight of a polyoxazoline polymer includes, for example, 1000, 1500, 2000, 2500, 3000, 3500, 4000, 4500, 5000, 5500, 6000, 6500, 7000, 7500, 8000, 10,000, 12,000, 20,000, 35,000, 40,000, 50,000, 60,000, 100,000, 200,000, 300,000, 400,000, or 500,000 Da. In some embodiments, a polyoxazoline polymer comprises poly(2-methyl 2-oxazoline) (PMOZ), poly(2-ethyl 2-oxazoline) (PEOZ), or poly(2-propyl 2-oxazoline) (PPOZ). In some cases, a cytokine (e.g., an interleukin, IFN, or TNF) polypeptide is conjugated to a polyoxazoline polymer.

In some embodiments, the water-soluble polymer is a polyacrylic acid polymer.

In some embodiments, the water-soluble polymer comprises polyamine. Polyamine is an organic polymer comprising two or more primary amino groups. In some embodiments, a polyamine includes a branched polyamine, a linear polyamine, or cyclic polyamine. In some cases, a polyamine is a low-molecular-weight linear polyamine. Exemplary polyamines include putrescine, cadaverine, spermidine, spermine, ethylene diamine, 1,3-diaminopropane, hexamethylenediamine, tetraethylmethylenediamine, and piperazine.

### Lipids

In some embodiments, the at least one additional moiety is a lipid. The lipid can be a fatty acid, e.g., a saturated fatty acid or an unsaturated fatty acid. Such fatty acids can have from 6 to 26 carbon atoms, from 6 to 24 carbon atoms, from 6 to 22 carbon atoms, from 6 to 20 carbon atoms, from 6 to 18 carbon atoms, from 20 to 26 carbon atoms, from 12 to 26 carbon atoms, from 12 to 24 carbon atoms, from 12 to 22 carbon atoms, from 12 to 20 carbon atoms, or from 12 to 18 carbon atoms in length. In some cases, the fatty acid has 6, 7, 8, 9, 10, 11, 12, 13, 14, 15, 16, 17, 18, 19, 20, 21, 22, 23, 24, 25, or 26 carbon atoms in length. Non-limiting examples of fatty acids include caproic acid (hexanoic acid), enanthic acid (heptanoic acid), caprylic acid (octanoic acid), pelargonic acid (nonanoic acid), capric acid (decanoic acid), undecylic acid (undecanoic acid), lauric acid (dodecanoic acid), tridecylic acid (tridecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), nonadecylic acid (nonadecanoic acid), arachidic acid (eicosanoic acid), heneicosylic acid (heneicosanoic acid), behenic acid (docosanoic acid), tricosylic acid (tricosanoic acid), lignoceric acid (tetracosanoic acid), pentacosylic acid (pentacosanoic acid), and cerotic acid (hexacosanoic acid). In some embodiments, the lipid binds to one or more serum proteins, thereby increasing serum stability and/or serum half-life.

### Proteins

In some embodiments, the at least one additional moiety described herein is a protein or a binding fragment thereof. Non-limiting examples of such proteins include albumin, transferrin, or transthyretin. In some embodiments, the protein or a binding fragment thereof comprises an antibody or a binding fragment thereof.

In some embodiments, the at least one additional moiety described herein is a tag useful for a function, such as purification or detection, which is extended at the N-terminus, the C-terminus, or both the N-terminus and the C-terminus of the polypeptide of the disclosure. In some embodiments, the tag may be a polyglutamate tag, a FLAG-tag, a HA-tag, a polyHis-tag (having about 5-10 histidines) (SEQ ID NO: 80), a hexahistidine tag (HHHHHH) (SEQ ID NO: 81), an 8X-His-tag (HHHHHHHH) (SEQ ID NO: 82), a Myc-tag, a Glutathione-S-transferase-tag, a green fluorescent protein-tag, Maltose binding protein-tag, a Thioredoxin-tag, or an Fc-tag. In some embodiments, the at least one additional moiety is an Fc-tag. In some specific embodiments, the Fe fragment is IgG, IgA, IgD, IgM, or IgE. In some specific embodiments, the IgG is IgG1, IgG2, IgG3, or IgG4. In some embodiments, the extension or additional moiety may be an N-terminal signal peptide fused to the protein to enhance expression. While such signal peptides are often cleaved during expression in the cell, some polypeptides may contain the cytokine with an intact signal peptide.

In some embodiments, the at least one additional moiety comprises an antigen-binding molecule. In specific embodiments, the antigen-binding molecule is an antibody or an antigen binding fragment thereof. In specific embodiments, the antigen-binding molecule is a multi-specific antigen-binding molecule. In specific cases, the antigen-binding molecule is a bispecific antibody or a bispecific antigen binding fragment thereof. In other specific cases, the antigen-binding molecule is antibody-drug conjugate (ADC). In other embodiments, the at least one additional moiety is selected from the group consisting of: scFv, (scFv)2, Fab, Fab′, F(ab′)2, Fv, dAb, Fd fragments, diabodies, F(ab′)3, disulfide linked Fv, sdAb (VHH or nanobody), CDR, di-scFv, bi-scFv, tascFv (tandem scFv), triabody, tetrabody, V-NAR domain, Fcab, IgGACH2, DVD-Ig, probody, a DARPin, a Centyrin, an affibody, an affilin, an affitin, an anticalin, an avimer, a Fynomer, a Kunitz domain peptide, a monobody (or adnectin), a tribody, and a nanofitin.

In other embodiments, the at least one additional moiety is a fragment that retain the ability to bind to the target antigen. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes); and a tetrabody (e.g., a tetramerized scFv, formed in a manner similar to a diabody, but in which four antigen-binding domains are created in a single complex; the four antigen binding domains may be directed towards the same or different epitopes).

In some embodiments, the antigen-binding molecule which is conjugated to the polypeptide described herein targets a tumor cell. In some embodiments, the antigen is a tumor-associated antigen. In specific embodiments, the tumor-associated antigen is selected from the group consisting of human epidermal growth factor receptor 2 (HER2), CD20, CD33, B-cell maturation antigen (BCMA), prostate-specific membrane (PSMA), DLL3, ganglioside GD2 (GD2), CD 123, anoctamin-1 (Anol), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD 19, CD30, CD38, CD40, CD52, CD70, CD79b, fis-like tyrosine kinase 3 (FLT3), glypican 3 (GPC3), B7 homolog 6 (B7H6), C— C chemokine receptor type 4 (CCR4), C—X—C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB-1), insulin like growth factor 1—receptor (IGF1R), and human epidermal growth factor receptor 3.

In some embodiments, the antigen-binding molecule which is conjugated to the polypeptide described herein targets a tumor cell. In some embodiments, the antigen is an immune-checkpoint antigen or an immune-checkpoint-associated antigen. In specific embodiments, the antigen is involved in an immune checkpoint pathway. In specific embodiments, the antigen is selected from the group consisting of: PD-1, PD-L1, TIGIT, CTLA- 4, PD-L2, B7-H3, B7-H4, BTLA, LAG3, CD112, CD112R, CD96, TIM-3, CD47, and CEACAM1. In specific embodiments, the antigen is a co-stimulatory immune checkpoint target. In specific embodiments, the antigen is selected from the group consisting of: CD155, ICOS, OX40, CD137, CD137L, CD27, CD28, and GITR.

In some embodiments, the at least one additional moiety is attached to C-terminus and/or N-terminus of the IL-21 variant. In some embodiments, the at least one additional moiety is attached to the IL-21 variant directly or via a linker.

In certain instances multiple additional moieties disclosed herein can be attached to a single polypeptide disclosed herein. In certain instances, multiple polypeptides disclosed herein can be attached to a single additional moiety disclosed herein. In certain instances a single additional moiety disclosed herein is attached to a single polypeptides disclosed herein. In some cases, the polypeptides to the additional moieties ratio is 1:1. In some cases, the polypeptides to the additional moieties ratio is about 2:1, 3:1, 4:1, 5:1, 6:1, 7:1, 8:1, 9:1, or 10:1. In some cases, the polypeptides to the additional moieties ratio is about 1:2, 1:3, 1:4, 1:5, 1:6, 1:7, 1:8, 1:9, or 1:10.

In some embodiments, the at least one additional moiety is albumin or a functional fragment thereof. Albumin is a family of water-soluble globular proteins. Albumin is commonly found in blood plasma, comprising about 55-60% of all plasma proteins. Human serum albumin (HSA) is a 585 amino acid polypeptide comprising three domains: domain I (amino acid residues 1-195), domain II (amino acid residues 196-383), and domain III (amino acid residues 384-585). Each domain further includes a binding site, which can interact either reversibly or irreversibly with endogenous ligands such as fatty acids, bilirubin, or hemin, or exogenous compounds, such as heterocyclic or aromatic compounds.

In some embodiments, the at least one additional moiety is transferrin. Transferrin is a 679 amino acid polypeptide that is about 80 kDa in size and comprises two Fe³⁺ binding sites with one at the N-terminal domain and the other at the C-terminal domain. Human transferrin has a half-life of about 7-12 days.

In some embodiments, the at least one additional moiety is transthyretin (TTR). Transthyretin is a transport protein located in the serum and cerebrospinal fluid which transports the thyroid hormone thyroxine (T4) and retinol-binding protein bound to retinol.

In some embodiments, the conjugates comprising the polypeptides described herein and the antibodies or the antigen binding fragments thereof described herein are enriched to related tumor tissues via the action of the antibodies or the antigen binding fragments thereof described herein. As such, the anti-tumor activity of the polypeptides described herein may be enhanced.

In some embodiments, the at least one additional moiety comprises a bioconjugate (e.g., a toll-like receptor (TLR) agonist such as a TLR1, TLR2, TLR3, TLR4, TLR5, TLR6, TLR7, TLR8, or TLR9 agonist; or a synthetic ligand such as Pam3Cys, CFA, MALP2, Pam2Cys, FSL-1, Hib-OMPC, Poly I:C, Poly A:U, AGP, MPLA, RC-529, MDF2p, CFA, or flagellin).

### Moieties Categorized by Functions

In some cases, the at least one additional moiety reduces IL-21 interaction with one or more IL-21 receptor domains or subunits. In some embodiments, the at least one additional moiety blocks IL-21 interaction with one or more IL-21 domains or subunits with its cognate receptor(s). In some cases, the at least one additional moiety increases serum half-life and/or improves stability in vivo.

### Masking Moieties

In some embodiments, the at least one additional moiety is a masking moiety. A masking moiety can block, occlude, inhibit, decrease, or otherwise reduce (i.e., masks) the activity or binding of IL-21 to its cognate receptor or protein. For example, the polypeptides disclosed herein can be activatable by a protease at a target site, such as in a tumor microenvironment, by inclusion of a proteolytically cleavable linker. In some embodiments, a proteolytically cleavable linker links the polypeptides to the masking moiety. Upon proteolytic cleavage of the cleavable linker at the target site, the polypeptides disclosed herein can become activated, thereby rendering the polypeptides capable of binding to its cognate receptor or protein.

A masking moiety can include a cleavable peptide and/or linker. In some embodiments, the cleavable linker comprises a cleavable peptide. A cleavable peptide is a polypeptide that includes a cleavage site, such as a protease cleavage site. The cleavage site is a site recognizable for cleavage of a portion of the cleavable peptide of a cleavable linker described herein. In some embodiments, the cleavable peptide comprises more than one cleavage site. In some embodiments, the cleavage site is an amino acid sequence that is recognized and cleaved by a cleaving agent. Exemplary cleaving agents include proteins, enzymes, DNAzymes, RNAzymes, metals, acids, and bases.

In some embodiments, the protease cleavage site is a matrix metalloprotease (MMP) cleavage site, a disintegrin and metalloprotease domain-containing (ADAM) metalloprotease cleavage site, a prostate specific antigen (PSA) protease cleavage site, a urokinase-type plasminogen activator (uPA) protease cleavage site, a membrane type serine protease 1 (MT-SPi) protease cleavage site, a matriptase protease cleavage site (ST14) or a legumain protease cleavage site. In embodiments, the matrix metalloprotease (MMP) cleavage site is a MMP9 cleavage site, a MMP13 cleavage site or a MMP2 cleavage site. In embodiments, the disintegrin and metalloprotease domain-containing (ADAM) metalloprotease cleavage site is an ADAM9 metalloprotease cleavage site, an ADAM10 metalloprotease cleavage site or an ADAM17 metalloprotease cleavage site. Protease cleavage sites can be designated by a specific amino acid sequence.

### Half-Life Extension Domains

In some embodiments, the at least one additional moiety is a half-life extension domain that increases serum half-life and/or improves stability of the polypeptides disclosed herein. The half-life extension domain can be fused to the N- or C-terminal of the polypeptides disclosed herein. In some embodiments, a proteolytically cleavable linker links the polypeptides disclosed herein to a half-life extension domain.

In some embodiments, the half-life extension domain is an albumin polypeptide or a functional fragment thereof. Albumin is a natural carrier protein that has an extended serum half-life of approximately three weeks due to its size and its susceptibility to FcRn-mediated recycling, which reduces the likelihood of intracellular degradation. Thus, linking an IL-21 polypeptide to albumin can significantly extend the serum half-life of the IL-21 polypeptide.

In some embodiments, an IL-21 polypeptide herein comprises an IL-21 mutein is fused to a heterologous polypeptide (i.e., a polypeptide that is not IL-21 and preferably is not a variant of IL-21, e.g., a conjugated moiety described herein). The heterologous polypeptide can increase the circulating half-life of the IL-21 polypeptide. For example, the polypeptide that increases the circulating half-life can be serum albumin, e.g., HSA.

In some embodiments, an IL-21 polypeptide provided herein includes an amino acid sequence that is heterologous to wildtype human IL-21 or a functional variant thereof. The amino acid sequence can be a half-life extension moiety that increases stability of the polypeptide by increasing in vivo half-life of the polypeptide, e.g., when administered to a subject. The half-life extension moiety can be a protein, an antibody, an albumin, an immunoglobulin or a fragment thereof, a transferrin, or a PEG. The antibody can be an anti-albumin antibody. The albumin can be a serum albumin, e.g., a mouse serum albumin or a human serum albumin. In some embodiments, the immunoglobulin fragment is the Fc domain of a human immunoglobulin, e.g., IgG1, IgG2, IgG3, IgG4, IgD, IgA, IgE, or IgM.

In some embodiments, the polypeptides disclosed herein comprises a half-life extension domain that comprises an albumin polypeptide, or a fragment or a variant thereof. In some embodiments, the albumin polypeptide is mouse serum albumin. In some embodiments, the albumin polypeptide is human serum albumin.

In some embodiments, the albumin polypeptide is linked to a masking moiety. In some embodiments, a masking moiety is linked to the N- or C-terminus of the albumin polypeptide. In some embodiments, the albumin polypeptide is linked to a masking moiety via a linker. In some embodiments, a linker is linked to the amino-terminus or the carboxy-terminus of the albumin polypeptide. In some embodiments, an N- or C-terminal spacer domain of the linker is linked to the N- or C-terminus of the albumin polypeptide. In some embodiments, a cleavable peptide of the linker is linked to the N- or C-terminus of the albumin polypeptide. In some embodiments, the albumin polypeptide is linked to a cytokine or functional fragment thereof (e.g., IL-21 or a mutein thereof). In some embodiments, a cytokine or functional fragment thereof is linked to the N- or C-terminus of the albumin polypeptide. In some embodiments, the albumin polypeptide is linked to a cytokine or functional fragment thereof via a linker. In some embodiments, a linker is linked to the amino-terminus or the carboxy-terminus of the albumin polypeptide.

In some embodiments, the conjugates comprises the polypeptides disclosed herein and a combination of two or more of the above-described moieties.

### Synergistic Effect

In some embodiments, the conjugates described herein exhibit better pharmacodynamic properties than the polypeptides described herein. In some embodiments, the conjugates described herein exhibit better pharmacodynamic properties than the at least one additional moiety. In specific embodiments, the conjugates described herein exhibit stronger anti-tumor activities than the polypeptides described herein. In some embodiments, the conjugates described herein exhibits stronger anti-tumor activities than the at least one additional moiety. In specific embodiments, the conjugates described herein lead to less side effects than the polypeptides described herein. In some embodiments, the conjugates described herein lead to less side effects than the at least one additional moiety.

### Thermal Stability

In some embodiments, the polypeptide or the conjugates described herein exhibit improved thermostability as compared to the wildtype human IL-21. In other embodiments, the polypeptide or the conjugates described herein are more stable in vitro as compared to the wildtype human IL-21. In other embodiments, the polypeptide or the conjugates described herein are more stable in vivo as compared to the wildtype human IL-21.

In some embodiments, the thermal stability of the polypeptides or the conjugates described herein is assessed by NanoTemper assay (Prometheus). In some embodiments, the thermal stability of the polypeptides or the conjugates described herein is assessed by qPCR equipped with the Protein Thermal Shift (PTS) research solution (Thermo Fisher). In some embodiments, the thermal stability of the polypeptides or the conjugates described herein is assessed by differential scanning calorimetry (DSC). In some embodiments, the thermal stability of the polypeptides or the conjugates described herein is assessed by differential scanning fluorimetry (DSF). In some specific embodiments, the melting temperature of the mutant IL21 polypeptide described herein is higher than the one of the wildtype IL21 equivalent. In some specific embodiments, the melting temperature of the mutant IL21 conjugates described herein is higher than the one of the wildtype IL21 equivalent.

### Property and Efficacy

In some embodiments, the polypeptide or the conjugates described herein exhibits any one or more, such as one, two, three, four, five, six, or seven of: (1) better pharmacodynamic properties than the wildtype IL-21 or wildtype IL-21 conjugates; (2) better pharmacodynamic properties than the at least one additional moiety; (3) stronger anti-tumor activities than the wildtype IL-21 or wildtype IL-21 conjugates; (4) stronger anti-tumor activities than the at least one additional moiety; (5) less side effects than the wildtype IL-21 or wildtype IL-21 conjugates; (6) synergistic effect in combination with at least one additional agent as compared to any of them administrated alone; and (7) improved thermostability as compared to the wildtype IL-21 or wildtype IL-21 conjugates.

### Nucleic Acid, Vector, and Transformed or Host Cells

In another aspect, provided herein are nucleic acid molecules encoding the polypeptides disclosed herein or the conjugates disclosed herein. In another aspect, provided herein are vectors comprising the nucleic acid molecules provided herein. In some embodiments, the vectors comprise a viral vector. In another aspect, provided herein are transformed or host cells that expresses the polypeptides disclosed herein or the conjugate disclosed herein.

The nucleic acid molecule can encode the polypeptide described herein. The nucleic acid molecule can be codon-optimized to encode the polypeptide described herein. Such a nucleic acid molecule can be inserted in an expression vector, which can then be transformed or incorporated into a host cell. The expression vector can be a plasmid. The host cell comprising the expression vector can be used to express the IL-21 mutant described herein. The host cell can be E. coli or other suitable bacterial cell.

Unless otherwise stated, the present invention can be performed using standard procedures known to one skilled in the art, for example, in Michael R. Green and Joseph Sambrook, Molecular Cloning: A Laboratory Manual, Cold Spring Harbor Laboratory Press, Cold Spring Harbor, N.Y., USA (2012); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Meolcular Biology, Vo. 203, 2003, Transgenic Mouse, editored by Marten H. Hofker and Jan van Deursen, which are all herein incorporated by reference in their entireties.

### Kits

In another aspect, provided herein are kits comprising the polypeptides, the conjugates, the nucleic acid molecules, the vectors, the transformed or host cells, or the pharmaceutical compositions, a combination thereof, and containers.

In some embodiments, provided herein are kits or articles of manufacture comprising the polypeptide described herein, including the pharmaceutical composition thereof. The kit can include instructions for use of a polypeptide such as in the methods provided herein. Thus, in some embodiments, the kit includes instructions for the use of a polypeptide in methods for treating a disorder described herein (e.g., a cancer) in a subject in need thereof by administering to the subject a therapeutically effective amount of the polypeptide. In some embodiments, the subject is a human. In some embodiments, the disorder is a cancer.

The kit can further include a container. Non-limiting examples of suitable containers include bottles, vials (e.g., dual chamber vials), syringes (such as single or dual chamber syringes), test tubes, and intravenous (IV) bags. The container can be formed from a variety of materials such as glass or plastic. The container can hold a formulation of an IL-21 polypeptide. In some embodiments, the formulation is a lyophilized formulation. In some embodiments, the formulation is a frozen formulation. In some embodiments, the formulation is a liquid formulation.

The kit may further comprise a label or a package insert, which is on or associated with the container, may indicate directions for reconstitution and/or use of the formulation. The label or package insert may further indicate that the formulation is useful or intended for intravenous, subcutaneous, or other modes of administration for treating a disorder (e.g., a cancer) in a subject. The container holding the formulation can be a single-use vial or a multi-use vial, which can allow for repeat administrations of the reconstituted formulation. The kit can further include a second container comprising a suitable diluent. The kit can further include other materials desirable from a commercial, therapeutic, and user standpoint, including other buffers, diluents, filters, needles, syringes, and package inserts with instructions for use.

In some embodiments, provided herein is a kit for a single dose-administration unit. Such a kit includes a container of an aqueous formulation of a therapeutic IL-21 polypeptide, including single or multi-chambered pre-filled syringes.

In some embodiments, provided herein are articles of manufacture or kits comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant, and the time of delivery is greater than a few moments.

### Pharmaceutical Compositions

In another aspect, provided herein are pharmaceutical compositions comprising the polypeptides disclosed herein or the conjugates disclosed herein, and pharmaceutically acceptable carriers. In some embodiments, the pharmaceutical composition of the disclosure can be a combination of a compound, e.g., the polypeptides described herein, with other chemical components, such as carriers, stabilizers, diluents, dispersing agents, suspending agents, thickening agents, and/or excipients. The pharmaceutical composition facilitates administration of the compound to an organism.

Pharmaceutical formulations for administration can include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. The suspension can also contain suitable stabilizers or agents which increase the solubility of a compound to allow for the preparation of highly concentrated solutions. The active ingredient can be in powder form for constitution with a suitable vehicle, for example, sterile pyrogen-free water, before use.

In practicing the methods of treatment or use provided herein, therapeutically effective amounts of a compound described herein is administered in a pharmaceutical composition to a subject having a disease or condition to be treated. In some embodiments, the subject is a mammal such as a human. A therapeutically effective amount can vary widely depending on the severity of the disease, the age and relative health of the subject, the potency of the compounds used, and other factors.

Pharmaceutical compositions can be formulated using one or more physiologically acceptable carriers comprising excipients and auxiliaries, which facilitate processing of the active compounds into preparations that can be used pharmaceutically. Formulation can be modified depending upon the route of administration chosen. Pharmaceutical compositions comprising compounds described herein can be manufactured, for example, by mixing, dissolving, emulsifying, encapsulating, entrapping, or compression processes.

The pharmaceutical compositions can include at least one pharmaceutically acceptable carrier, diluent, or excipient and compounds described herein as free-base or pharmaceutically acceptable salt form. Pharmaceutical compositions can contain solubilizers, stabilizers, tonicity enhancing agents, buffers, and preservatives.

Methods for the preparation of compositions comprising the compounds described herein include formulating the compounds with one or more inert, pharmaceutically acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the disclosure can be, for example, an immediate release form or a controlled release formulation. An immediate release formulation can be formulated to allow the compounds to act rapidly following administration. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements, or has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically effective blood profile) over about 4, about 8, about 12, about 16, or about 24 hours.

A compound described herein can be conveniently formulated into pharmaceutical compositions composed of one or more pharmaceutically acceptable carriers. See e.g., Remington's Pharmaceutical Sciences, latest edition, by E. W. Martin Mack Pub. Co., Easton, PA, incorporated by reference in its entirety, which discloses pharmaceutically acceptable excipients and carriers, and methods of preparing pharmaceutical compositions. Such carriers can be carriers for administration of compositions to humans and non-humans, including solutions such as sterile water, saline, and buffered solutions at physiological pH. Pharmaceutical compositions can also include one or more additional active ingredients such as antimicrobial agents, anti-inflammatory agents, and anesthetics.

Pharmaceutical formulations can include additional carriers, as well as thickeners, diluents, buffers, preservatives, and surface active agents in addition to the agents disclosed herein.

A pharmaceutical composition disclosed herein can be administered in a therapeutically effective amount by various forms and routes including, for example, oral, topical, parenteral, intravenous injection, intravenous infusion, subcutaneous injection, subcutaneous infusion, intramuscular injection, intramuscular infusion, intradermal injection, intradermal infusion, intraperitoneal injection, intraperitoneal infusion, intracerebral injection, intracerebral infusion, subarachnoid injection, subarachnoid infusion, intraocular injection, intraspinal injection, intrasternal injection, ophthalmic administration, endothelial administration, local administration, intranasal administration, intrapulmonary administration, rectal administration, intraarterial administration, intrathecal administration, inhalation, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, or intrasternal administration.

A pharmaceutical composition can be administered in a local manner, for example, via injection of the compound directly into an organ, optionally in a depot or sustained release formulation or implant. A pharmaceutical composition can be provided in the form of a rapid release formulation, in the form of an extended release formulation, or in the form of an intermediate release formulation. A rapid release form can provide an immediate release. An extended release formulation can provide a controlled release or a sustained delayed release.

A compound herein may be administered in combination with one or more therapeutic agents, for example, a cytokine, antiviral agent, or antifungal agent. The term “therapeutic agent” encompasses any agent administered to treat a symptom or disease in an animal in need of such treatment. The compound can also be administered as a component of a vaccine, i.e., combined with essentially any preparation intended for active immunological prophylaxis.

Toxicity and therapeutic efficacy of a compound herein can be determined by standard pharmaceutical procedures in cell culture or experimental animals. Cell culture assays and animal studies can be used to determine the LD50 (the dose lethal to 50% of a population) and the ED50 (the dose therapeutically effective in 50% of a population). The dose ratio between toxic and therapeutic effects is the therapeutic index, which can be expressed as the ratio LD50/ED50). A compound herein that exhibits large therapeutic indices are preferred. The data obtained from these cell culture assays and animal studies can be used in formulating a range of dosages suitable for use in humans. The dosage of such compounds lies preferably within a range of circulating concentrations that include the ED50 with little or no toxicity. The dosage may vary within this range depending upon a variety of factors, e.g., the dosage form employed, the route of administration utilized, the condition of the subject, and the like.

A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful doses in humans. Levels in plasma may be measured, for example, by HPLC. The exact formulation, route of administration and dosage can be chosen by the individual physician in view of the patient's condition.

An attending physician for patients treated with a compound herein would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

A compound herein can be administered to an individual alone as a pharmaceutical preparation appropriately formulated for the route of delivery and for the condition being treated. Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, and the like. For transmucosal administration, penetrants appropriate to the barrier to be permeated can be used in the formulation.

A compound herein can be formulated as a liquid with carriers that may include a buffer and or salt such as phosphate buffered saline. Alternatively, a compound herein may be formulated as a solid with carriers or fillers such as lactose, binders such as starches, and/or lubricants such as talc or magnesium stearate and, optionally, stabilizers.

For oral delivery, the formulated end product may be a tablet, pill, capsule, dragee, liquid, gel, syrup, slurry, suspension, and the like. Also, push-fit capsules made of gelatin, as well as soft, sealed capsules made of gelatin and a plasticizer, such as glycerol or sorbitol may be used. The push-fit capsules can contain the active ingredients in admixture with fillers as above while in soft capsules, the active compounds may be dissolved or suspended in suitable liquids, such as fatty oils, liquid paraffin, or liquid polyethylene glycols.

Formulation for oral delivery can involve conventional mixing, dissolving, granulating, dragee-making, levigating, emulsifying, encapsulating, entrapping, lyophilizing processes, and the like. A compound herein also may be mixed with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, sorbitol, and the like; cellulose preparations such as, for example, maize starch, wheat starch, rice starch, potato starch, gelatin, gum tragacanth, methyl cellulose, hydroxypropylmethylcellulose, sodium carboxymethylcellulose, polyvinylpyrrolidone (PVP), and the like, as well as mixtures of any two or more thereof. If desired, disintegrating agents may be added, such as cross-linked polyvinyl pyrrolidone, agar, alginic acid or a salt thereof such as sodium alginate, and the like.

If injection is desired, a compound herein can be formulated in aqueous solutions, preferably in physiologically compatible buffers such as Hank's solution, Ringer's solution, or physiological saline buffer. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions.

### Methods

In another aspect, provided herein are methods of preparing the polypeptides described herein or the conjugates described herein, comprising: (a) constructing the nucleic acid molecules and the vectors described herein; (b) culturing the transformed or host cells described herein; and (c) harvesting the polypeptides from the transformed or host cells.

In another aspect, provided herein are methods of treating a subject in need thereof, comprising administering to the subject the pharmaceutical compositions described herein in an amount effective to treat the subject. In some embodiments, the subject has a solid tumor.

In some embodiments, the subject described herein has or is suspected to have a proliferative disease or condition. In some embodiments, the proliferative disease or condition is a neoplastic disease, such as cancer. In some cases, the cancer is a metastatic cancer. In some cases, the cancer is a relapsed or refractory cancer. In some embodiments, the cancer is a treatment-naïve cancer. The treatment-naive cancer can be a cancer that has not been treated by a therapy.

In some embodiments, the cancer is leukemia, lymphoma, sarcoma, myeloma, glioma, glioblastoma, glioblastoma multiforme, glioma, head and neck cancer, colorectal cancer, colon cancer, prostate cancer, castration-resistant prostate cancer, pancreatic cancer, melanoma, breast cancer (e.g., triple negative, ER positive, ER negative, chemotherapy resistant, trastuzumab-resistant, HER2 positive, doxorubicin resistant, tamoxifen resistant, ductal carcinoma, lobular carcinoma, primary, metastatic), neuroblastoma, lung cancer (e.g., non-small cell lung carcinoma, squamous cell lung carcinoma (e.g., head, neck, or esophagus), adenocarcinoma, large cell lung carcinoma, small cell lung carcinoma, carcinoid, sarcoma), ovarian cancer, bone cancer (e.g., osteosarcoma, chondrosarcoma, Ewing sarcoma), bladder cancer, cervical cancer, liver cancer (e.g., hepatocellular carcinoma), kidney cancer, skin cancer, testicular cancer, adrenal cancer, adenoid cystic carcinoma, anal cancer, brain cancer, ductal carcinoma, endometrial cancer, esophageal cancer, gastric cancer, oral cancer, thyroid cancer, retinoblastoma, parathyroid cancer, pituitary cancer, bile duct cancer, uterine cancer, acute myeloid leukemia, lymphoma, B cell lymphoma, multiple myeloma, mesothelioma, medulloblastoma, Hodgkin's Disease, Non-Hodgkin's Lymphoma, neuroblastoma, rhabdomyosarcoma, primary thrombocytosis, primary macroglobulinemia, primary brain tumors, malignant pancreatic insulinoma, malignant carcinoid, urinary bladder cancer, premalignant skin lesions, neuroblastoma, genitourinary tract cancer, malignant hypercalcemia, adrenal cortical cancer, neoplasms of the endocrine or exocrine pancreas, medullary thyroid cancer, medullary thyroid carcinoma, papillary thyroid cancer, hepatocellular carcinoma, Paget's disease of the nipple, phyllodes tumors, lobular carcinoma, ductal carcinoma, cancer of the pancreatic stellate cells, or cancer of the hepatic stellate cells. Additional non-limiting examples of cancers include, cancer of the thyroid, endocrine system, brain, breast, cervix, colon, head and neck, esophagus, liver, kidney, ovary, stomach, or uterus.

In some embodiments, the cancer is a solid tumor. Non-limiting examples of solid tumors include bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, and prostate cancer.

In some embodiments, the cancer is a hematologic malignancy, such as a leukemia, a lymphoma, or a myeloma. In some cases, the hematologic malignancy is a T cell malignancy. In other cases, the hematological malignancy is a B cell malignancy. Non-limiting examples of hematologic malignancies include chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B cell lymphoma (PMBL), immunoblastic large cell lymphoma, precursor B lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the subjects described herein have or are suspected to have an inflammatory or autoimmune disease. In some embodiments, the inflammatory or autoimmune disease is atherosclerosis, obesity, inflammatory bowel disease (IBD), rheumatoid arthritis, allergic encephalitis, psoriasis, atopic skin disease, osteoporosis, peritonitis, hepatitis, lupus, celiac disease, Sjögren's syndrome, polymyalgia rheumatica, multiple sclerosis (MS), ankylosing spondylitis, type 1 diabetes mellitus, alopecia areata, vasculitis, and temporal arteritis, graft versus host disease (GVHD), asthma, COPD, a paraneoplastic autoimmune disease, cartilage inflammation, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, seronegative enthesopathy and arthropathy (SEA) syndrome, juvenile dermatomyositis, juvenile psoriatic arthritis, juvenile scleroderma, juvenile systemic lupus erythematosus, juvenile vasculitis, pauciarticular rheumatoid arthritis, systemic onset rheumatoid arthritis, enteropathic arthritis, reactive arthritis, Reiter's syndrome, dermatomyositis, psoriatic arthritis, scleroderma, vasculitis, myolitis, polymyolitis, dermatomyolitis, polyarteritis nodossa, Wegener's granulomatosis, arteritis, ploymyalgia rheumatica, sarcoidosis, sclerosis, primary biliary sclerosis, sclerosing cholangitis, psoriasis, plaque psoriasis, guttate psoriasis, inverse psoriasis, pustular psoriasis, erythrodermic psoriasis, dermatitis, atopic dermatitis, atherosclerosis, Still's disease, Systemic Lupus Erythematosus (SLE), myasthenia gravis, Crohn's disease, ulcerative colitis, celiac disease, rhinosinusitis, rhinosinusitis with polyps, eosinophilic esophogitis, eosinophilic bronchitis, Guillain-Barre disease, thyroiditis (e.g., Graves' disease), Addison's disease, Raynaud's phenomenon, autoimmune hepatitis, transplantation rejection, kidney damage, or hepatitis C-induced vasculitis.

### EXAMPLES

### Example 1: Recombinant Expression and Preparation of IL-21 Variants

Full-length amino acid sequences of the IL-21 variants are shown in TABLE 1. Alignments between the variants and the wildtype are shown in TABLE 2.

TABLE 1
Full-length amino acid sequences of the IL-21 variants

| SEQ | | | |
|---|---|---|---|
| ID | | | |
| NO. | ID | Amino Acid Sequence | Notes |
| 1 | Wildtype | QGQDRHMIRMRQLIDIVDQLKNYVNDLVPEFLPAPEDV | 30^{th} to 162^{nd} amino |
| | | ETNCEWSAFSCFQKAQLKSANTGNNERIINVSIKKLKRK | acid (without |
| | | PPSTNAGRRQKHRLTCPSCDSYEKKPPKEFLERFKSLLQ | signal peptide), |
| | | KMIHQHLSSRTHGSEDS | mature human IL- |
| | | | 21 (isoform 1) |
| 2 | wildtype | QGQDRHMIRMRQLIDIVDQLKNYVNDLVPEFLPAPEDV | 30^{th} to 153^{rd} amino |
| | | ETNCEWSAFSCFQKAQLKSANTGNNERIINVSIKKLKRK | acid (without |
| | | PPSTNAGRRQKHRLTCPSCDSYEKKPPKEFLERFKSLLQ | signal peptide), |
| | | KVSTLSFI | mature human IL- |
| | | | 21 (isoform 2) |
| 3 | Full | MRSSPGNMERIVICLMVIFLGTLVHKSSSQGQDRHMIRM | human IL-21 with |
| | wildtype | RQLIDIVDQLKNYVNDLVPEFLPAPEDVETNCEWSAFSC | signal peptide |
| | | FQKAQLKSANTGNNERIINVSIKKLKRKPPSTNAGRRQK | (isoform 1) |
| | | HRLTCPSCDSYEKKPPKEFLERFKSLLQKMIHQHLSSRT | |
| | | HGSEDS | |
| 4 | Full | MRSSPGNMERIVICLMVIFLGTLVHKSSSQGQDRHMIRM | human IL-21 with |
| | wildtype | RQLIDIVDQLKNYVNDLVPEFLPAPEDVETNCEWSAFSC | signal peptide |
| | | FQKAQLKSANTGNNERIINVSIKKLKRKPPSTNAGRRQK | (isoform 2) |
| | | HRLTCPSCDSYEKKPPKEFLERFKSLLQKVSTLSFI | |
| 5 | m155 | QGQDRHMIRMRQLIDIVDQLKNYVNDLVPEFLPAPEDV | P79E |
| | | ETNCEWSAFSCFQKAQLKSANTGNNERIINVSIKKLKRK | |
| | | PESTNAGRRQKHRLTCPSCDSYEKKPPKEFLERFKSLLQ | |
| | | KMIHQHLSSRTHGSEDS | |
| 6 | m156 | QGQDRHMIRMRQLIDIVDQLKNYVNDLVPEFLPAPEDV | STNAGRRQKHR |
| | | ETNCEWSAFSCFQKAQLKSANTGNNERIINVSIKKLKRK | (SEQ ID NO: 71) |
| | | PPGGGSEGGGSLTCPSCDSYEKKPPKEFLERFKSLLQKM | change to |
| | | IHQHLSSRTHGSEDS | GGGSEGGGS |
| | | | (SEQ ID NO: 72) |
| 7 | m157 | QGQDCHMIRMRQLIDIVDQLKNYVNDLVPEFLPAPEDV | R5C, P79C |
| | | ETNCEWSAFSCFQKAQLKSANTGNNERIINVSIKKLKRK | |
| | | PCSTNAGRRQKHRLTCPSCDSYEKKPPKEFLERFKSLLQ | |
| | | KMIHQHLSSRTHGSEDS | |
| 8 | m158 | QGQDRCMIRMRQLIDIVDQLKNYVNDLVPEFLPAPEDV | H6C, P79C |
| | | ETNCEWSAFSCFQKAQLKSANTGNNERIINVSIKKLKRK | |
| | | PCSTNAGRRQKHRLTCPSCDSYEKKPPKEFLERFKSLLQ | |
| | | KMIHQHLSSRTHGSEDS | |
| 9 | m103 | QGQDRHMIRMRQLIDIVDQLKNYVNDLVPEFLPAPEDV | P79E + STNAGRR |
| | | ETNCEWSAFSCFQKAQLKSANTGNNERIINVSIKKLKRK | QKHR (SEQ ID |
| | | PEGGGSEGGGSLTCPSCDSYEKKPPKEFLERFKSLLQKM | NO: |
| | | IHQHLSSRTHGSEDS | 71)/GGGSEGGGS |
| | | | (SEQ ID NO: 72) |
| 10 | m159 | QGQDRCMIRMRQLIDIVDQLKNYVNDLVPEFLPAPEDV | H6C, P79C + STNA |
| | | ETNCEWSAFSCFQKAQLKSANTGNNERIINVSIKKLKRK | GRRQKHR (SEQ |
| | | PCGGGSEGGGSLTCPSCDSYEKKPPKEFLERFKSLLQKM | ID NO: |
| | | IHQHLSSRTHGSEDS | 71)/GGGSEGGGS |
| | | | (SEQ ID NO: 72) |
| 11 | m160 | QGQDRHMIRMRQLIDIVDQLKNYVNDLVPEFLPAPEDV | K73Y |
| | | ETNCEWSAFSCFQKAQLKSANTGNNERIINVSIKYLKRK | |
| | | PPSTNAGRRQKHRLTCPSCDSYEKKPPKEFLERFKSLLQ | |
| | | KMIHQHLSSRTHGSEDS | |
| 12 | m161 | QGQDRHMIRMRQLIDIVDQLKNYVNDLVPEFLPAPEDV | K73Y, P79E |
| | | ETNCEWSAFSCFQKAQLKSANTGNNERIINVSIKYLKRK | |
| | | PESTNAGRRQKHRLTCPSCDSYEKKPPKEFLERFKSLLQ | |
| | | KMIHQHLSSRTHGSEDS | |
| 13 | m162 | QGQDRCMIRMRQLIDIVDQLKNYVNDLVPEFLPAPEDV | K73Y, H6C, P79C |
| | | ETNCEWSAFSCFQKAQLKSANTGNNERIINVSIKYLKRK | |
| | | PCSTNAGRRQKHRLTCPSCDSYEKKPPKEFLERFKSLLQ | |
| | | KMIHQHLSSRTHGSEDS | |
| 14 | m163 | QGQDRHMIRMRQLIDIVDQLKNYVNDLVPEFLPAPEDV | K73Y, P79E + STN |
| | | ETNCEWSAFSCFQKAQLKSANTGNNERIINVSIKYLKRK | AGRRQKHR |
| | | PEGGGSEGGGSLTCPSCDSYEKKPPKEFLERFKSLLQKM | (SEQ ID NO: |
| | | IHQHLSSRTHGSEDS | 71)/GGGSEGGGS |
| | | | (SEQ ID NO: 72) |
| 15 | m164 | QGQDRHMIRMRQLIDIVDQLKNYVNDLVPEFLPAPEDV | K73F |
| | | ETNCEWSAFSCFQKAQLKSANTGNNERIINVSIKFLKRK | |
| | | PPSTNAGRRQKHRLTCPSCDSYEKKPPKEFLERFKSLLQ | |
| | | KMIHQHLSSRTHGSEDS | |
| 16 | m165 | QGQDRHMIRMRQLIDIVDQLKNYVNDLVPEFLPAPEDV | K73F, P79E |
| | | ETNCEWSAFSCFQKAQLKSANTGNNERIINVSIKFLKRK | |
| | | PESTNAGRRQKHRLTCPSCDSYEKKPPKEFLERFKSLLQ | |
| | | KMIHQHLSSRTHGSEDS | |
| 17 | m166 | QGQDRCMIRMRQLIDIVDQLKNYVNDLVPEFLPAPEDV | K73F, H6C, P79C |
| | | ETNCEWSAFSCFQKAQLKSANTGNNERIINVSIKFLKRK | |
| | | PCSTNAGRRQKHRLTCPSCDSYEKKPPKEFLERFKSLLQ | |
| | | KMIHQHLSSRTHGSEDS | |
| 18 | m167 | QGQDRHMIRMRQLIDIVDQLKNYVNDLVPEFLPAPEDV | K73F, P79E + STN |
| | | ETNCEWSAFSCFQKAQLKSANTGNNERIINVSIKFLKRK | AGRRQKHR |
| | | PEGGGSEGGGSLTCPSCDSYEKKPPKEFLERFKSLLQKM | (SEQ ID NO: |
| | | IHQHLSSRTHGSEDS | 71)/GGGSEGGGS |
| | | | (SEQ ID NO: 72) |
| 19 | m131 | QGQDRHMIRMRQLIDIVDQLKNYVNDLVPEFLPAPEDV | S70L, K73Y |
| | | ETNCEWSAFSCFQKAQLKSANTGNNERIINVLIKYLKRK | |
| | | PPSTNAGRRQKHRLTCPSCDSYEKKPPKEFLERFKSLLQ | |
| | | KMIHQHLSSRTHGSEDS | |
| 20 | m99 | QGQDRHMIRMRQLIDIVDQLKNYVNDLVPEFLPAPEDV | S70L, K73Y, P79E |
| | | ETNCEWSAFSCFQKAQLKSANTGNNERIINVLIKYLKRK | |
| | | PESTNAGRRQKHRLTCPSCDSYEKKPPKEFLERFKSLLQ | |
| | | KMIHQHLSSRTHGSEDS | |
| 21 | m133 | QGQDRCMIRMRQLIDIVDQLKNYVNDLVPEFLPAPEDV | S70L, K73Y, P79C, |
| | | ETNCEWSAFSCFQKAQLKSANTGNNERIINVLIKYLKRK | H6C |
| | | PCSTNAGRRQKHRLTCPSCDSYEKKPPKEFLERFKSLLQ | |
| | | KMIHQHLSSRTHGSEDS | |
| 22 | m145 | QGQDRHMIRMRQLIDIVDQLKNYVNDLVPEFLPAPEDV | S70L, K73Y, P79E |
| | | ETNCEWSAFSCFQKAQLKSANTGNNERIINVLIKYLKRK | + STNAGRRQKH |
| | | PEGGGSEGGGSLTCPSCDSYEKKPPKEFLERFKSLLQKM | R (SEQ ID NO: |
| | | IHQHLSSRTHGSEDS | 71)/GGGSEGGGS |
| | | | (SEQ ID NO: 72) |
| 23 | m151 | QGQDRHMIRMRQLIDIVDQLKNYVNDLVPEFLPAPEDV | S70L, K72S, K73Y |
| | | ETNCEWSAFSCFQKAQLKSANTGNNERIINVLISYLKRK | |
| | | PPSTNAGRRQKHRLTCPSCDSYEKKPPKEFLERFKSLLQ | |
| | | KMIHQHLSSRTHGSEDS | |
| 24 | m100 | QGQDRHMIRMRQLIDIVDQLKNYVNDLVPEFLPAPEDV | S70L, K72S, K73Y, |
| | | ETNCEWSAFSCFQKAQLKSANTGNNERIINVLISYLKRK | P79E |
| | | PESTNAGRRQKHRLTCPSCDSYEKKPPKEFLERFKSLLQ | |
| | | KMIHQHLSSRTHGSEDS | |
| 25 | m146 | QGQDRHMIRMRQLIDIVDQLKNYVNDLVPEFLPAPEDV | S70L, K72S, K73Y, |
| | | ETNCEWSAFSCFQKAQLKSANTGNNERIINVLISYLKRK | P79E + STNAGRR |
| | | PEGGGSEGGGSLTCPSCDSYEKKPPKEFLERFKSLLQKM | QKHR (SEQ ID |
| | | IHQHLSSRTHGSEDS | NO: |
| | | | 71)/GGGSEGGGS |
| | | | (SEQ ID NO: 72) |
| 26 | m72 | QGQDRHMIRMRQLIDRVDQLKNYVNDLVPEFLPAPEDV | I16R, S70L, K73Y; |
| | | ETNCEWSAFSCFQKAQLKSANTGNNERIINVLIKYLKRK | |
| | | PPSTNAGRRQKHRLTCPSCDSYEKKPPKEFLERFKSLLQ | |
| | | KMIHQHLSSRTHGSEDS | |
| 27 | m74 | QGQDRHMIRMRQLILRVDQLKNYVNDLVPEFLPAPEDV | D15L, I16R, S70L, |
| | | ETNCEWSAFSCFQKAQLKSANTGNNERIINVLIKYLKRK | K73Y |
| | | PPSTNAGRRQKHRLTCPSCDSYEKKPPKEFLERFKSLLQ | |
| | | KMIHQHLSSRTHGSEDS | |
| 28 | m76 | QGQDRHMIRMRQLIKWVDQLKNYVNDLVPEFLPAPED | D15K, I16W, S70L, |
| | | VETNCEWSAFSCFQKAQLKSANTGNNERIINVLIKYLKR | K73Y; |
| | | KPPSTNAGRRQKHRLTCPSCDSYEKKPPKEFLERFKSLL | |
| | | QKMIHQHLSSRTHGSEDS | |
| 29 | m94 | QGQDRHMIRMRQLIRWVDQLKNYVNDLVPEFLPAPED | D15R, I16W, S70L, |
| | | VETNCEWSAFSCFQKAQLKSANTGNNERIINVLIKYLKR | K73Y |
| | | KPPSTNAGRRQKHRLTCPSCDSYEKKPPKEFLERFKSLL | |
| | | QKMIHQHLSSRTHGSEDS | |
| 30 | m102 | QGQDRHMIRMRQLILRVDQLKNYVNDLVPEFLPAPEDV | D15L, I16R, S70L, |
| | | ETNCEWSAFSCFQKAQLKSANTGNNERIINVLIKYLKRK | K73Y;P79E |
| | | PESTNAGRRQKHRLTCPSCDSYEKKPPKEFLERFKSLLQ | |
| | | KMIHQHLSSRTHGSEDS | |
| 31 | m111 | QGQDRHMIRMRQLIRWVDQLKNYVNDLVPEFLPAPED | D15R, I16W, S70L, |
| | | VETNCEWSAFSCFQKAQLKSANTGNNERIINVLIKYLKR | K73Y, P79E + STN |
| | | KPEGGGSEGGGSLTCPSCDSYEKKPPKEFLERFKSLLQK | AGRRQKHR |
| | | MIHQHLSSRTHGSEDS | (SEQ ID NO: |
| | | | 71)/GGGSEGGGS |
| | | | (SEQ ID NO: 72) |
| 32 | m137 | QGQDRCMIRMRQLILRVDQLKNYVNDLVPEFLPAPEDV | D15L, I16R, S70L, |
| | | ETNCEWSAFSCFQKAQLKSANTGNNERIINVLIKYLKRK | K73Y, P79C, H6C |
| | | PCSTNAGRRQKHRLTCPSCDSYEKKPPKEFLERFKSLLQ | |
| | | KMIHQHLSSRTHGSEDS | |
| 33 | m138 | QGQDRHMICMRQLILRVDQLKNYVNDLVPEFLPAPEDV | D15L, I16R, S70L, |
| | | ETNCEWSAFSCFQKAQLKSANTGNNERIINVLIKYLKRK | K73Y, P79C, R9C |
| | | PCSTNAGRRQKHRLTCPSCDSYEKKPPKEFLERFKSLLQ | |
| | | KMIHQHLSSRTHGSEDS | |
| 34 | m140 | QGQDRHMIRMRQLIKWVDQLKNYVNDLVPEFLPAPED | D15K, I16W, S70L, |
| | | VETNCEWSAFSCFQKAQLKSANTGNNERIINVLISYLKR | K72S, K73Y |
| | | KPPSTNAGRRQKHRLTCPSCDSYEKKPPKEFLERFKSLL | |
| | | QKMIHQHLSSRTHGSEDS | |
| 35 | m139 | QGQDRHMIRMRQLILRVDQLKNYVNDLVPEFLPAPEDV | D15L, I16R, S70L, |
| | | ETNCEWSAFSCFQKAQLKSANTGNNERIINVLIKYLKRK | K73Y, P79E + STN |
| | | PEGGGSEGGGSLTCPSCDSYEKKPPKEFLERFKSLLQKM | AGRRQKHR |
| | | IHQHLSSRTHGSEDS | (SEQ ID NO: |
| | | | 71)/GGGSEGGGS |
| | | | (SEQ ID NO: 72) |
| 36 | m154 | QGQDRHMIRMRQLIDIVDQLKNYVNDLVPEFLPAPEDV | S70L, K73F |
| | | ETNCEWSAFSCFQKAQLKSANTGNNERIINVLIKFLKRK | |
| | | PPSTNAGRRQKHRLTCPSCDSYEKKPPKEFLERFKSLLQ | |
| | | KMIHQHLSSRTHGSEDS | |
| 37 | m88 | QGQDRHMIRMRQLIDIVDQLKNYVNDLVPEFLPAPEDV | S70L, K73F, P79E; |
| | | ETNCEWSAFSCFQKAQLKSANTGNNERIINVLIKFLKRK | |
| | | PESTNAGRRQKHRLTCPSCDSYEKKPPKEFLERFKSLLQ | |
| | | KMIHQHLSSRTHGSEDS | |
| 38 | m152 | QGQDRCMIRMRQLIDIVDQLKNYVNDLVPEFLPAPEDV | S70L, K73F, P79C, |
| | | ETNCEWSAFSCFQKAQLKSANTGNNERIINVLIKFLKRK | H6C |
| | | PCSTNAGRRQKHRLTCPSCDSYEKKPPKEFLERFKSLLQ | |
| | | KMIHQHLSSRTHGSEDS | |
| 39 | m153 | QGQDRHMIRMRQLIDIVDQLKNYVNDLVPEFLPAPEDV | S70L, K73F, P79E + |
| | | ETNCEWSAFSCFQKAQLKSANTGNNERIINVLIKFLKRK | STNAGRRQKHR |
| | | PEGGGSEGGGSLTCPSCDSYEKKPPKEFLERFKSLLQKM | (SEQ ID NO: |
| | | IHQHLSSRTHGSEDS | 71)/GGGSEGGGS |
| | | | (SEQ ID NO: 72) |
| 40 | m98 | QGQDRHMIRMRQLIDIVDQLKNYVNDLVPEFLPAPEDV | S70L, K72S, K73F, |
| | | ETNCEWSAFSCFQKAQLKSANTGNNERIINVLISFLKRKP | P79E |
| | | ESTNAGRRQKHRLTCPSCDSYEKKPPKEFLERFKSLLQK | |
| | | MIHQHLSSRTHGSEDS | |
| 41 | m144 | QGQDRHMIRMRQLIDIVDQLKNYVNDLVPEFLPAPEDV | S70L, K72S, K73F, |
| | | ETNCEWSAFSCFQKAQLKSANTGNNERIINVLISFLKRKP | P79E + STNAGRR |
| | | EGGGSEGGGSLTCPSCDSYEKKPPKEFLERFKSLLQKMI | QKHR (SEQ ID |
| | | HQHLSSRTHGSEDS | NO: |
| | | | 71)/GGGSEGGGS |
| | | | (SEQ ID NO: 72) |
| 42 | m19 | QGQDRHMIRMRQLIDIVDQLKNYVNDLVPEFLPAPEDV | S70A, K72S, K73F; |
| | | ETNCEWSAFSCFQKAQLKSANTGNNERIINVAISFLKRK | |
| | | PPSTNAGRRQKHRLTCPSCDSYEKKPPKEFLERFKSLLQ | |
| | | KMIHQHLSSRTHGSEDS | |
| 43 | m141 | QGQDRHMIRMRQLIKIVDQLKNYVNDLVPEFLPAPEDV | D15K, S70L, K73F, |
| | | ETNCEWSAFSCFQKAQLKSANTGNNERIINVLIKFLKRK | P79E + STNAGRR |
| | | PEGGGSEGGGSLTCPSCDSYEKKPPKEFLERFKSLLQKM | QKHR (SEQ ID |
| | | IHQHLSSRTHGSEDS | NO: |
| | | | 71)/GGGSEGGGS |
| | | | (SEQ ID NO: 72) |
| 44 | m142 | QGQDRHMIRMRQLIRIVDQLKNYVNDLVPEFLPAPEDV | D15R, S70L, K73F, |
| | | ETNCEWSAFSCFQKAQLKSANTGNNERIINVLIKFLKRK | P79E + STNAGRR |
| | | PEGGGSEGGGSLTCPSCDSYEKKPPKEFLERFKSLLQKM | QKHR (SEQ ID |
| | | IHQHLSSRTHGSEDS | NO: |
| | | | 71)/GGGSEGGGS |
| | | | (SEQ ID NO: 72) |
| 45 | m143 | QGQDRHMIRMRQLIDWVDQLKNYVNDLVPEFLPAPED | I16W, S70L, K73F, |
| | | VETNCEWSAFSCFQKAQLKSANTGNNERIINVLIKFLKR | P79E + STNAGRR |
| | | KPEGGGSEGGGSLTCPSCDSYEKKPPKEFLERFKSLLQK | QKHR (SEQ ID |
| | | MIHQHLSSRTHGSEDS | NO: |
| | | | 71)/GGGSEGGGS |
| | | | (SEQ ID NO: 72) |
| 46 | m89 | QGQDRHMIRMRQAIDIVDQLKNYVNDLVPEFLPAPEDV | L13A, S70L, K73F, |
| | | ETNCEWSAFSCFQKAQLKSANTGNNERIINVLIKFLKRK | P79E; |
| | | PESTNAGRRQKHRLTCPSCDSYEKKPPKEFLERFKSLLQ | |
| | | KMIHQHLSSRTHGSEDS | |
| 47 | m95 | QGQDRHMIRMRQLIKIVDQLKNYVNDLVPEFLPAPEDV | D15K, S70L, K73F, |
| | | ETNCEWSAFSCFQKAQLKSANTGNNERIINVLIKFLKRK | P79E |
| | | PESTNAGRRQKHRLTCPSCDSYEKKPPKEFLERFKSLLQ | |
| | | KMIHQHLSSRTHGSEDS | |
| 48 | m96 | QGQDRHMIRMRQLIRIVDQLKNYVNDLVPEFLPAPEDV | D15R, S70L, K73F, |
| | | ETNCEWSAFSCFQKAQLKSANTGNNERIINVLIKFLKRK | P79E |
| | | PESTNAGRRQKHRLTCPSCDSYEKKPPKEFLERFKSLLQ | |
| | | KMIHQHLSSRTHGSEDS | |
| 49 | m97 | QGQDRHMIRMRQLIDWVDQLKNYVNDLVPEFLPAPED | I16W, S70L, K73F, |
| | | VETNCEWSAFSCFQKAQLKSANTGNNERIINVLIKFLKR | P79E |
| | | KPESTNAGRRQKHRLTCPSCDSYEKKPPKEFLERFKSLL | |
| | | QKMIHQHLSSRTHGSEDS | |
| 50 | m92 | QGQDRHMIRMRQLIDWVDQLKNYVNDLVPEFLPAPED | I16W, S70L, K72S, |
| | | VETNCEWSAFSCFQKAQLKSANTGNNERIINVLISFLKR | K73F |
| | | KPPSTNAGRRQKHRLTCPSCDSYEKKPPKEFLERFKSLL | |
| | | QKMIHQHLSSRTHGSEDS | |
| 51 | m127 | QGQDCHMIRMRQLIDIVDQLKNYVNDLVPEFLPAPEDV | R5C, R76F, P79C |
| | | ETNCEWSAFSCFQKAQLKSANTGNNERIINVSIKKLKFK | |
| | | PCSTNAGRRQKHRLTCPSCDSYEKKPPKEFLERFKSLLQ | |
| | | KMIHQHLSSRTHGSEDS | |
| 52 | m128 | QGQDRCMIRMRQLIDIVDQLKNYVNDLVPEFLPAPEDV | H6C, R76F, P79C |
| | | ETNCEWSAFSCFQKAQLKSANTGNNERIINVSIKKLKFK | |
| | | PCSTNAGRRQKHRLTCPSCDSYEKKPPKEFLERFKSLLQ | |
| | | KMIHQHLSSRTHGSEDS | |
| 53 | m86 | QGQDRHMIRMRQLIKWVDQLKNYVNDLVPEFLPAPED | D15K, I16W, S70L, |
| | | VETNCEWSAFSCFQKAQLKSANTGNNERIINVLIKKLKF | R76F |
| | | KPPSTNAGRRQKHRLTCPSCDSYEKKPPKEFLERFKSLL | |
| | | QKMIHQHLSSRTHGSEDS | |
| 54 | m87 | QGQDRHMIRMRQLIRWVDQLKNYVNDLVPEFLPAPED | D15R, I16W, S70L, |
| | | VETNCEWSAFSCFQKAQLKSANTGNNERIINVLIKKLKF | R76F |
| | | KPPSTNAGRRQKHRLTCPSCDSYEKKPPKEFLERFKSLL | |
| | | QKMIHQHLSSRTHGSEDS | |
| 55 | m129 | QGQDRHMICMRQLIRIVDQLKNYVNDLVPEFLPAPEDV | D15R, |
| | | ETNCEWSAFSCFQKAQLKSANTGNNERIINVSIKKLKRK | P78G, R9C, P79C |
| | | GCSTNAGRRQKHRLTCPSCDSYEKKPPKEFLERFKSLLQ | |
| | | KMIHQHLSSRTHGSEDS | |
| 56 | m109 | QGQDRHMIRMRQLIKWVDQLKNYVNDLVPEFLPAPED | D15K, I16W, S70L, |
| | | VETNCEWSAFSCFQKAQLKSANTGNNERIINVLIKKLKF | R76F, P79E |
| | | KPESTNAGRRQKHRLTCPSCDSYEKKPPKEFLERFKSLL | |
| | | QKMIHQHLSSRTHGSEDS | |
| 57 | m110 | QGQDRHMIRMRQLIRWVDQLKNYVNDLVPEFLPAPED | D15R, I16W, S70L, |
| | | VETNCEWSAFSCFQKAQLKSANTGNNERIINVLIKKLKF | R76F, P79E |
| | | KPESTNAGRRQKHRLTCPSCDSYEKKPPKEFLERFKSLL | |
| | | QKMIHQHLSSRTHGSEDS | |
| 58 | m121 | QGQDRHMIRMRQLIRIVDQLKNYVNDLVPEFLPAPEDV | D15R, |
| | | ETNCEWSAFSCFQKAQLKSANTGNNERIINVSIKKLKFK | R76F, P78E, P79E |
| | | EESTNAGRRQKHRLTCPSCDSYEKKPPKEFLERFKSLLQ | |
| | | KMIHQHLSSRTHGSEDS | |
| 59 | m29 | QGQDRHMIRMRWLIDIVDQLKNYVNDLVPEFLPAPEDV | Q12W, S70A, R76F |
| | | ETNCEWSAFSCFQKAQLKSANTGNNERIINVAIKKLKFK | |
| | | PPSTNAGRRQKHRLTCPSCDSYEKKPPKEFLERFKSLLQ | |
| | | KMIHQHLSSRTHGSEDS | |
| 60 | m30 | QGQDRHMIRMRQLIKIVDQLKNYVNDLVPEFLPAPEDV | D15K, S70A, R76F; |
| | | ETNCEWSAFSCFQKAQLKSANTGNNERIINVAIKKLKFK | |
| | | PPSTNAGRRQKHRLTCPSCDSYEKKPPKEFLERFKSLLQ | |
| | | KMIHQHLSSRTHGSEDS | |
| 61 | m31 | QGQDRHMIRMRQLILIVDQLKNYVNDLVPEFLPAPEDV | D15L, S70A, R76F; |
| | | ETNCEWSAFSCFQKAQLKSANTGNNERIINVAIKKLKFK | |
| | | PPSTNAGRRQKHRLTCPSCDSYEKKPPKEFLERFKSLLQ | |
| | | KMIHQHLSSRTHGSEDS | |
| 62 | m32 | QGQDRHMIRMRQLIRIVDQLKNYVNDLVPEFLPAPEDV | D15R, S70A, R76F; |
| | | ETNCEWSAFSCFQKAQLKSANTGNNERIINVAIKKLKFK | |
| | | PPSTNAGRRQKHRLTCPSCDSYEKKPPKEFLERFKSLLQ | |
| | | KMIHQHLSSRTHGSEDS | |

Note:
in the Examples and figures disclosed herein, IL-21 variants with a Fc-tag are labeled as “m(ID)-Fc”, IL-21 variants with a His-tag are labeled as “m(ID)-His”.

TABLE 2
Alignments of IL-21 variants with wildtype IL-21

| | Pos. | | | | | | | | | | | | | | | | | | | | | | | | SEQ |
|---|---|---|---|---|---|---|---|---|---|---|---|---|---|---|---|---|---|---|---|---|---|---|---|---|---|
| ID | 5 | 6 | 9 | 12 | 13 | 15 | 16 | 70 | 72 | 73 | 76 | 78 | 79 | 80 | 81 | 82 | 83 | 84 | 85 | 86 | 87 | 88 | 89 | 90 | ID NO: |
| WT IL-21 | R | H | R | Q | L | D | I | S | K | K | R | P | P | S | T | N | A | G | R | R | Q | K | H | R | 83 |
| m155 | | | | | | | | | | | | | E | | | | | | | | | | | | 84 |
| m156 | | | | | | | | | | | | | | GGGSEGGGS | | | | | | | | | | | 85 |
| m157 | C | | | | | | | | | | | | C | | | | | | | | | | | | 86 |
| m158 | | C | | | | | | | | | | | C | | | | | | | | | | | | 86 |
| m103 | | | | | | | | | | | | | E | GGGSEGGGS | | | | | | | | | | | 87 |
| m159 | | C | | | | | | | | | | | C | GGGSEGGGS | | | | | | | | | | | 88 |
| m160 | | | | | | | | | | Y | | | | | | | | | | | | | | | 83 |
| m161 | | | | | | | | | | Y | | | E | | | | | | | | | | | | 84 |
| m162 | | C | | | | | | | | Y | | | C | | | | | | | | | | | | 86 |
| m163 | | | | | | | | | | Y | | | E | GGGSEGGGS | | | | | | | | | | | 87 |
| m164 | | | | | | | | | | F | | | | | | | | | | | | | | | 83 |
| m165 | | | | | | | | | | F | | | E | | | | | | | | | | | | 84 |
| m166 | | C | | | | | | | | F | | | C | | | | | | | | | | | | 86 |
| m167 | | | | | | | | | | F | | | E | GGGSEGGGS | | | | | | | | | | | 87 |
| m131 | | | | | | | | L | | Y | | | | | | | | | | | | | | | 83 |
| m99 | | | | | | | | L | | Y | | | E | | | | | | | | | | | | 84 |
| m133 | | C | | | | | | L | | Y | | | C | | | | | | | | | | | | 86 |
| m145 | | | | | | | | L | | Y | | | E | GGGSEGGGS | | | | | | | | | | | 87 |
| m151 | | | | | | | | L | S | Y | | | | | | | | | | | | | | | 83 |
| m100 | | | | | | | | L | S | Y | | | E | | | | | | | | | | | | 84 |
| m146 | | | | | | | | L | S | Y | | | E | GGGSEGGGS | | | | | | | | | | | 87 |
| m72 | | | | | | | R | L | | Y | | | | | | | | | | | | | | | 83 |
| m74 | | | | | | L | R | L | | Y | | | | | | | | | | | | | | | 83 |
| m76 | | | | | | K | W | L | | Y | | | | | | | | | | | | | | | 83 |
| m94 | | | | | | R | W | L | | Y | | | | | | | | | | | | | | | 83 |
| m102 | | | | | | L | R | L | | Y | | | E | | | | | | | | | | | | 84 |
| m111 | | | | | | R | W | L | | Y | | | E | GGGSEGGGS | | | | | | | | | | | 87 |
| m137 | | C | | | | L | R | L | | Y | | | C | | | | | | | | | | | | 86 |
| m138 | | | C | | | L | R | L | | Y | | | C | | | | | | | | | | | | 86 |
| m140 | | | | | | K | W | L | S | Y | | | | | | | | | | | | | | | 83 |
| m139 | | | | | | L | R | L | | Y | | | E | GGGSEGGGS | | | | | | | | | | | 87 |
| m154 | | | | | | | | L | | F | | | | | | | | | | | | | | | 83 |
| m88 | | | | | | | | L | | F | | | E | | | | | | | | | | | | 84 |
| m152 | | C | | | | | | L | | F | | | C | | | | | | | | | | | | 86 |
| m153 | | | | | | | | L | | F | | | E | GGGSEGGGS | | | | | | | | | | | 87 |
| m98 | | | | | | | | L | S | F | | | E | | | | | | | | | | | | 84 |
| m144 | | | | | | | | L | S | F | | | E | GGGSEGGGS | | | | | | | | | | | 87 |
| m19 | | | | | | | | A | S | F | | | | | | | | | | | | | | | 83 |
| m141 | | | | | | K | | L | | F | | | E | GGGSEGGGS | | | | | | | | | | | 87 |
| m142 | | | | | | R | | L | | F | | | E | GGGSEGGGS | | | | | | | | | | | 87 |
| m143 | | | | | | | W | L | | F | | | E | GGGSEGGGS | | | | | | | | | | | 87 |
| m89 | | | | | A | | | L | | F | | | E | | | | | | | | | | | | 84 |
| m95 | | | | | | K | | L | | F | | | E | | | | | | | | | | | | 84 |
| m96 | | | | | | R | | L | | F | | | E | | | | | | | | | | | | 84 |
| m97 | | | | | | | W | L | | F | | | E | | | | | | | | | | | | 84 |
| m92 | | | | | | | W | L | S | F | | | | | | | | | | | | | | | 83 |
| m127 | C | | | | | | | | | | F | | C | | | | | | | | | | | | 86 |
| m128 | | C | | | | | | | | | F | | C | | | | | | | | | | | | 86 |
| m86 | | | | | | K | W | L | | | F | | | | | | | | | | | | | | 83 |
| m87 | | | | | | R | W | L | | | F | | | | | | | | | | | | | | 83 |
| m129 | | | C | | | R | | | | | | G | C | | | | | | | | | | | | 89 |
| m109 | | | | | | K | W | L | | | F | | E | | | | | | | | | | | | 84 |
| m110 | | | | | | R | W | L | | | F | | E | | | | | | | | | | | | 84 |
| m121 | | | | | | R | | | | | F | E | E | | | | | | | | | | | | 90 |
| m29 | | | | W | | | | A | | | | | | | | | | | | | | | | | 83 |
| m30 | | | | | | K | | A | | | | | | | | | | | | | | | | | 83 |
| m31 | | | | | | L | | A | | | F | | | | | | | | | | | | | | 83 |
| m32 | | | | | | R | | A | | | | | | | | | | | | | | | | | 83 |

Note:
in the Examples and figures disclosed herein, IL-21 variants with a Fc-tag are labeled as “m(ID)-Fc”, IL-21 variants with a His-tag are labeled as “m(ID)-His”.

The exemplary WT IL-21 and IL-21 variants are shown in TABLE 1 and TABLE 2. They were generated with a Fe-tag by adding human IgG1 Fe (SEQ ID NO. 63) directly to the C-terminus of each WT IL-21 and IL-21 variants, and adding a 18-aa signal peptide (SEQ ID NO. 64) directly at the N-terminus of each mature WT IL-21 and IL-21 variants (the signal peptide was added to promote expression in mammalian cell lines and subsequently cleaved in the expression product).

The sequence of IgG1 Fc is (from N- to C-terminus):

(SEQ ID NO. 63)
EPKSADKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSREEMTKNQVSLTCLVKGFYPSDIAVEWESNGQPENNYKTTP
PVLDSDGSFFLYSKLTVDKSRWQQGNVFSCSVMHEALHNHYTQKS
LSLSPGK

The sequence of the 18-aa signal peptide is (from N- to C-terminus):

(SEQ ID NO. 64)
MHSSALLCCLVLLTGVRA

An exemplary amino acid sequence of encoded IL-21-Fc construct is (from N- to C-terminus):

MHSSALLCCLVLLTGVRAQGQDRHMIRMRQLIDIVDQLKNYVNDL
VPEFLPAPEDVETNCEWSAFSCFQKAQLKSANTGNNERIINVSIK
KLKRKPPSTNAGRRQKHRLTCPSCDSYEKKPPKEFLERFKSLLQK
MIHQHLSSRTHGSEDSEPKSADKTHTCPPCPAPELLGGPSVFLFP
PKPKDTLMISRTPEVTCVVVDVSHEDPEVKFNWYVDGVEVHNAKT
KPREEQYNSTYRVVSVLTVLHQDWLNGKEYKCKVSNKALPAPIEK
TISKAKGQPREPQVYTLPPSREEMTKNQVSLTCLVKGFYPSDIAV
EWESNGQPENNYKTTPPVLDSDGSFFLYSKLTVDKSRWQQGNVFS
CSVMHEALHNHYTQKSLSLSPGK

(Amino acid sequence of WT IL-21-human IgG1 Fc construct (SEQ ID NO. 65), wherein the segment in bold represents the 18-aa signal peptide; the underlined segment represents the mature IL-21 sequence; the italic part represents human IgG1-Fe-tag)

All variants share the above construct structure with same signal peptide and Fe-tag sequences, but each variant had its unique mature IL-21 sequences as shown in TABLE 1 and TABLE 2.

### 1) Construction of Expression Plasnid.

Based on the amino acid sequence of encoded IL-21-Fc constructs (wildtype IL-21 and IL-21 variants), the DNA sequence was synthesized and confirmed by gene sequencing.

An exemplary expression sequence of IL-21-Fe construct is (from N- to C-terminus):

[GCGGCCGC]ᵢ[AAACTACAAGACAGACTTGCAAAAGAAGGC]ᵢᵢ
[ATGCACAGCTCAGCACTGCTCTGTTGCCTGGTCCTCCTGACTGG
GGTGAGGGCC]ᵢᵢᵢ[CAGGGACAAGACAGACACATGATCAGAATG
AGACAACTGATCGACATAGTGGACCAACTCAAAAACTACGTCAAC
GACCTCGTCCCCGAGTTCCTCCCCGCCCCAGAGGACGTCGAGACC
AACTGCGAATGGAGCGCCTTCAGCTGCTTCCAGAAAGCCCAGCTC
AAGTCCGCCAACACCGGCAACAACGAAAGAATCATCAACGTGAGC
ATCAAAAAGCTGAAGAGAAAACCCCCCTCCACCAACGCCGGCAGA
AGACAGAAGCACAGACTGACCTGCCCCTCCTGCGACAGCTACGAA
AAAAAGCCACCCAAGGAGTTCCTCGAAAGATTCAAATCCCTGCTC
CAAAAAATGATCCACCAACACCTCAGCAGCAGAACCCACGGCTCC
GAAGACTCC]ᵢᵥ[GAGCCCAAATCTGCTGACAAAACTCACACATG
CCCACCGTGCCCAGCACCTGAACTCCTGGGGGGACCGTCAGTCTT
CCTCTTCCCCCCAAAACCCAAGGACACCCTCATGATCTCCCGGAC
CCCCGAGGTCACATGCGTGGTGGTGGACGTGAGCCACGAAGACCC
TGAGGTCAAGTTCAACTGGTACGTGGACGGCGTGGAGGTGCATAA
TGCCAAGACAAAGCCGCGGGAGGAGCAGTACAACAGCACGTACCG
TGTGGTCAGCGTCCTCACCGTCCTGCACCAGGACTGGCTGAATGG
CAAGGAGTACAAGTGCAAGGTCTCCAACAAAGCCCTCCCAGCCCC
CATCGAGAAAACCATCTCCAAAGCCAAAGGGCAGCCCCGAGAACC
ACAGGTGTACACCCTGCCCCCATCCCGGGAGGAGATGACCAAGAA
CCAGGTCAGCCTGACCTGCCTGGTCAAAGGCTTCTATCCCAGCGA
CATCGCCGTGGAGTGGGAGAGCAATGGGCAGCCGGAGAACAACTA
CAAGACCACGCCTCCCGTGCTGGACTCCGACGGCTCCTTCTTCCT
CTACAGCAAGCTCACCGTGGACAAGAGCAGGTGGCAGCAGGGGAA
CGTCTTCTCATGCTCCGTGATGCATGAGGCTCTGCACAACCACTA
CACGCAGAAGAGCCTCTCCCTGTCTCCGGGTAAA]ᵥ[TGA]ᵥᵢ[T
TCTAGA]ᵥᵢᵢ

(WT IL-21-Fe DNA expression sequence (SEQ ID NO. 66), wherein Segment (i) represents a NotI endonuclease cleavable site; Segment (ii) represents a Okaxaki fragment element; the Segment (iii) in bold represents the 18-aa signal peptide encoding sequence; the underlined Segment (iv) represents the mature IL-21 encoding sequence; the italic Segment (v) represents human IgG1-Fc-tag encoding sequence; Segment (vi) represents a stop codon; and Segment (vii) represents a Xbal endonuclease cleavable site).

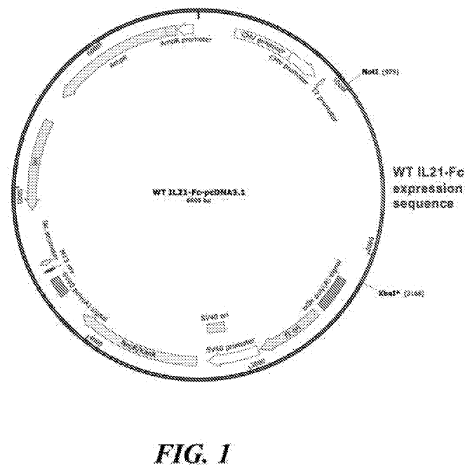

The confirmed DNA sequence was constructed into the expression vector pcDNA3.1 (Thermo Fisher, catalog no. V79020). The plasmid containing the IL-21-Fc gene (an exemplary WT IL-21 recombinant expression vector shown in FIG. 1) was transformed into E. coli DH5a cells. A large amount of both wildtype-Fc and variant-Fc plasmids was obtained by culturing the E. coli DH5α cells for amplification and plasmid purification. The preparation of plasmid constructs was operated following the protocols from Michael R. Green and Joseph Sambrook, Molecular Cloning: A Laboratory Manual, Cold Spring Harbor Laboratory Press, Cold Spring Harbor, N.Y., USA (4^{th} edition, 2012), which is also herein incorporated by reference in its entirety.

### 2) Expression of Wildtype IL-21-Fc and IL-21 Variant-Fc Proteins in HEK293F Cells.

Cells were inoculated in 300 mL of medium (Gibco™ FreeStyle™ 293 expression medium, catalog no. 12338018) in a 1-L shake flask at an inoculation volume of 0.5×10⁶ cells/ml. The cells were incubated for 24 hr in a shaker incubator at 37° C., 120 rpm, and 5% carbon dioxide concentration, until the cell density reached 1×10⁶ cells/ml. Then, 300 μg of wildtype-Fc or variant-Fc expression plasmid was added to 30 ml of medium. The mixture was vortexed for 3s to thoroughly mix. About 1.2 ml (0.5 mg/ml) of transfection reagent PEI MAX 40K (Polysciences, catalog no. 24765) was added to the transfection reagent/plasmid mixture. The mixture was held static for 20 min, and then added to the HEK293F cells. After transfection, the cells were incubated for 48-54 hr in a shaker incubator at 37° C., 120 rpm, and 5% carbon dioxide concentration. After incubation, the supernatant of the culture medium was isolated by centrifugation and prepared for protein purification.

### 2) Purification and Detection of Wildtype IL-21-Fc and IL-21 Variant-Fc Proteins.

The wildtype IL-21-Fc and IL-21 variant-Fe proteins were purified using Protein A agarose microspheres. An appropriate amount of Protein A agarose microspheres were added to the supernatant in the previous step, and incubated at 4° C. for 10 min. The wildtype IL-21-Fc and IL-21 variant-Fe proteins that were attached to the Protein A agarose microspheres were collected by centrifuging the mixture with a low speed (1000 rpm) for 3 minutes. An appropriate volume of wash buffer (50 mM PBS, pH 7.4) was then added to remove non-specific proteins. Finally, an appropriate volume of elution buffer (Glycine-HCl, pH 3) was added to elute the wildtype-Fc or variants-Fe off the microspheres, and the pH was adjusted to 7.4, thus the purified wildtype-Fc or variants-Fc were obtained.

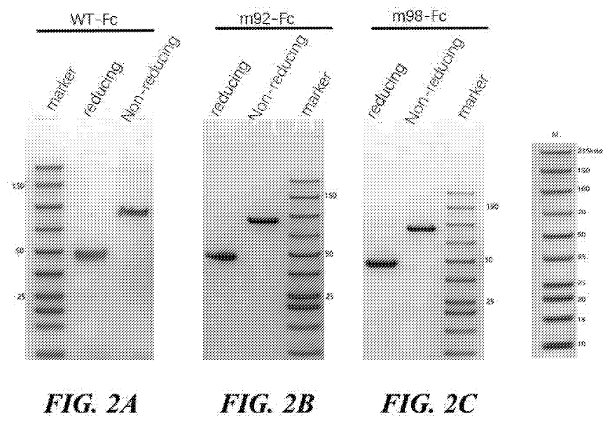

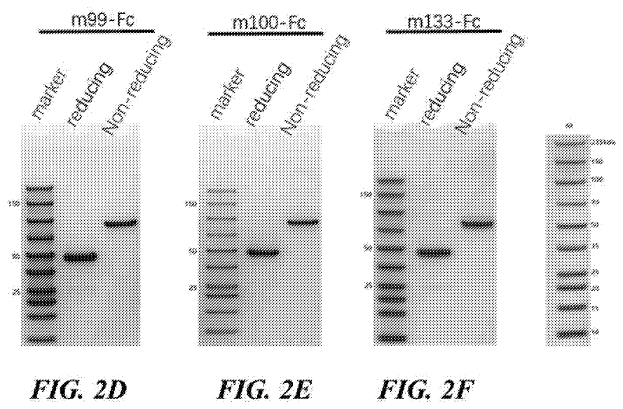

Wildtype IL-21 and IL-21 variants designed in TABLE 1 were prepared in IL-21-Fc form. The purified wildtype IL-21-Fc and IL-21 variant-Fc proteins were detected by SDS-PAGE protein gel. As representative results, the expression data of wild-type IL-21-Fe, m92-Fe, m98-Fc, m99-Fe, m100-Fc and m133-Fc are shown in TABLE 3. The data show that these IL-21 variant-Fc proteins had higher expression than WT IL21-Fc. SDS-PAGE protein gel detection results show that the sizes of WT IL-21-Fc, m92-Fc, m98-Fc, m99-Fe, m100-Fe, and m133-Fc were consistent with the theoretical molecular weight, for both their reducing samples and non-reducing samples (see FIGS. 2A-2F).

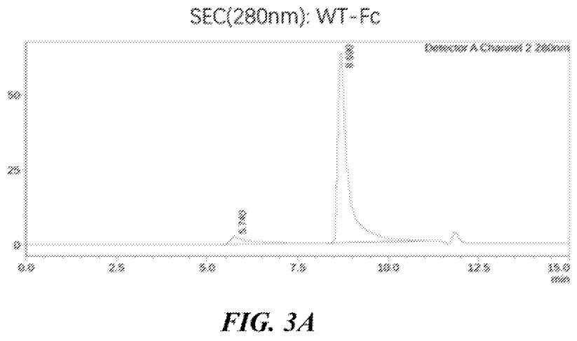

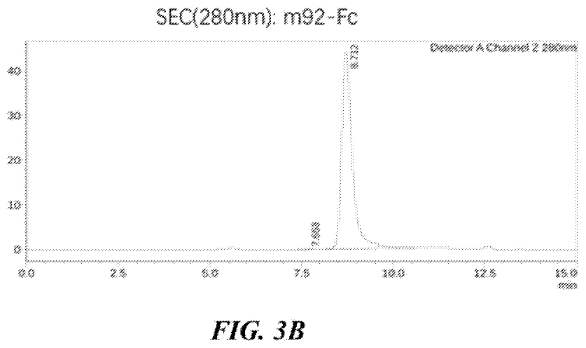

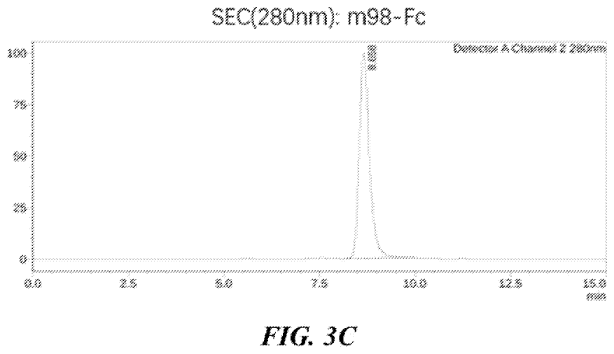

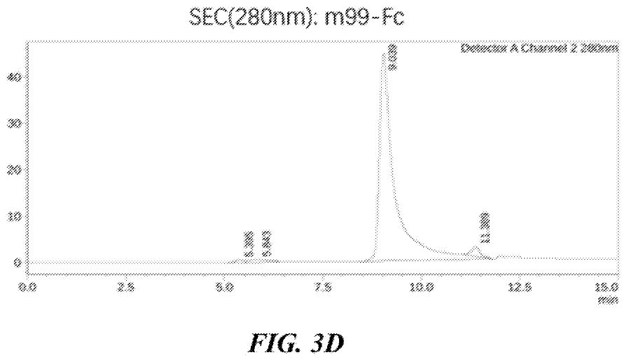

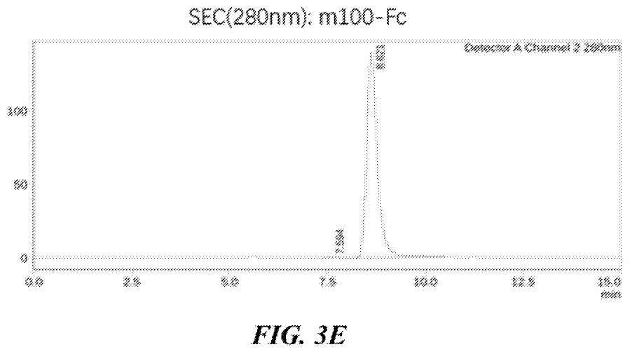

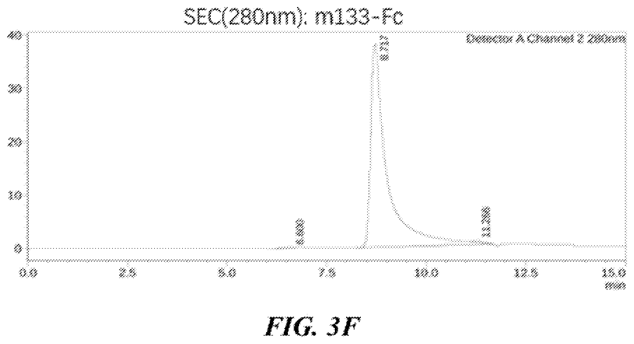

The purity and protein aggregation status of wild-type IL-21-Fc and IL-21 variant-Fc proteins were further detected by size exclusion chromatography (SEC). SEC results at 280 nm showed that the wild-type IL-21-Fc, m92-Fc, m98-Fc, m99-Fc, m100-Fc and m133-Fc peaked all around 8.5 to 9 min. The data indicate that these proteins were monomeric protein, and no aggregated proteins were produced (see FIGS. 3A-3F).

TABLE 3
Yield of the purified wildtype IL-21-Fc and IL-21 variant-Fc

| Name | Substitutions | Expression (mg/L) |
|---|---|---|
| WT IL21-Fc | NA | 7.6 |
| m92-Fc | I16W, S70L, K72S, K73F | 30.7 |
| m98-Fc | S70L, K72S, K73F, P79E | 88.8 |
| m99-Fc | S70L, K73Y, P79E | 111.6 |
| m100-Fc | S70L, K72S, K73Y, P79E | 103.7 |
| m133-Fc | S70L, K73Y, P79C, H6C | 124.5 |

### Alternative Preparation 1

The WT IL-21 or IL-21 variants of the disclosure was also prepared and purified using human IgG4 Fc.

Full-length amino acid sequences of the IL-21 variants are shown in TABLE 1. Alignments between the variants and the wildtype are shown in TABLE 2. The exemplary WT IL-21 and IL-21 variants are shown in TABLE 1 and TABLE 2. They were generated with a Fc-tag by fusing a human IgG4 Fc (SEQ ID NO. 68) to the C-terminus of each WT IL-21 and IL-21 variants, and further adding an 18-aa signal peptide (SEQ ID NO. 64) directly at the N-terminus of each mature WT IL-21 and IL-21 variants (the signal peptide was added to promote expression in mammalian cell lines and subsequently cleaved in the expression product).

Sequence of IgG4 Fc (from N- to C-terminus):

(SEQ ID NO. 68)
ESKYGPPCPSCPAPEFLGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLGK

The sequence of the 18-aa signal peptide is (from N- to C-terminus):

(SEQ ID NO. 64)
MHSSALLCCLVLLTGVRA

An exemplary amino acid sequence of encoded IL-21-IgG4 Fc construct is (from N- to C-terminus):

MHSSALLCCLVLLTGVRAQGQDRHMIRMRQLIDIVDQLKNYVNDL
VPEFLPAPEDVETNCEWSAFSCFQKAQLKSANTGNNERIINVSIK
KLKRKPPSTNAGRRQKHRLTCPSCDSYEKKPPKEFLERFKSLLQK
MIHQHLSSRTHGSEDSESKYGPPCPSCPAPEFLGGPSVFLFPPKP
KDTLMISRTPEVTCVVVDVSQEDPEVQFNWYVDGVEVHNAKTKPR
EEQFNSTYRVVSVLTVLHQDWLNGKEYKCKVSNKGLPSSIEKTIS
KAKGQPREPQVYTLPPSQEEMTKNQVSLTCLVKGFYPSDIAVEWE
SNGQPENNYKTTPPVLDSDGSFFLYSRLTVDKSRWQEGNVFSCSV
MHEALHNHYTQKSLSLSLGK

(Amino acid sequence of WT IL-21-IgG4 Fc construct (SEQ ID NO. 69), wherein the segment in bold represents the 18-aa signal peptide; the underlined segment represents the mature IL-21 sequence; the italic part represents human IgG4 Fe-tag).

All variants share the above construct structure with same signal peptide and IgG4 Fc-tag sequences, but each variant had its unique mature IL-21 sequences as shown in TABLE 1 and TABLE 2.

### 1) Construction of Expression Plasmid.

Based on the amino acid sequence of wildtype IL-21-IgG4 Fc and IL-21 variant-IgG4 Fc, the DNA sequence was synthesized and confirmed by gene sequencing. The confirmed DNA sequence was constructed into the expression vector pcDNA3.1 (Thermo Fisher, catalog no. V79020). The plasmid containing the IL-21-IgG4 Fc gene was transformed into E. coli DH5a cells. A large amount of both wildtype and variant plasmids were obtained by culturing the E. coli DH5α cells for amplification and plasmid purification.

### 2) Expression of Wildtype IL-21-IgG4 Fc and IL-21 Variant-IgG4 Fc Proteins in CHO Cells.

CHO Cells(CHO-K1,Taizhou Biointron Biological, Inc.) were inoculated in 30 mL of medium (Taizhou Biointron Biological, Inc.) in a 100 mL shake flask at an inoculation volume of 0.5×10⁶ cells/ml. The cells were incubated for 24 hr in a shaker incubator at 37° C., 120 rpm, and 5% carbon dioxide concentration, until the cell density reached 1.5×10⁶ cells/ml. Then, 60 μg of wildtype-IgG4 Fc or variant-IgG4 Fc expression plasmid was added to 1 ml of medium. The mixture was vortexed for 3s to thoroughly mix. About 15 μl of transfection reagent (Taizhou Biointron Biological, Inc.) was added to was added to 1 ml of medium. The plasmid mixture was added to the transfection reagent mixture. The transfection reagent/plasmid mixture was held static for 20 min, and then added to the CHO cells. After transfection, the cells were incubated for 48-54 hr in a shaker incubator at 37° C., 120 rpm, and 5% carbon dioxide concentration. After incubation, the supernatant of the culture medium was isolated by centrifugation and prepared for protein purification.

### 3) Purification and Detection of Wildtype IL-21-IgG4 Fc and IL-21 Variant-IgG4 Fc Proteins.

The wildtype IL-21-IgG4 Fc and IL-21 variant-IgG4 Fc proteins were purified using Protein A agarose microspheres. An appropriate amount of Protein A agarose microspheres were added to the supernatant in the previous step, and incubated at 4° C. for 10 min. The wildtype IL- 21-IgG4 Fc and IL-21 variant-IgG4 Fc proteins that were attached to the Protein A agarose microspheres were collected by centrifuging the mixture with a low speed (1000 rpm) for 3 minutes. An appropriate volume of wash buffer (50 mM PBS, pH 7.4) was then added to remove non-specific proteins. Finally, an appropriate volume of elution buffer (Glycine-HCl, pH 3) was added to elute the wildtype-IgG4 Fc or variants-IgG4 Fc off the microspheres, and the pH was adjusted to 7.4, thus the purified wildtype-IgG4 Fc or variants-IgG4 Fc were obtained.

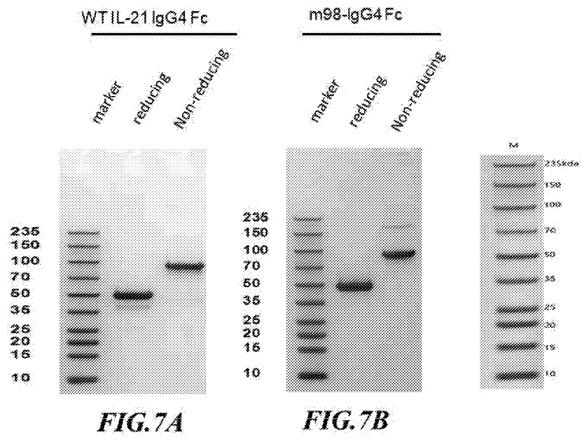

Wildtype IL-21 and IL-21 variants designed in TABLE 1 were prepared in IL-21-IgG4 Fe form. The purified wildtype IL-21-IgG4 Fc and IL-21 variant-IgG4 Fc proteins were detected by SDS-PAGE protein gel. The quantification is shown in TABLE 7. The data show that m98-IgG4 Fc protein had higher expression than WT IL-21-IgG4 Fe. SDS-PAGE protein gel detection results show that the sizes of WT IL-21-IgG4 Fc and m98-IgG4 Fc were consistent with the theoretical molecular weight, for both their reducing samples and non-reducing samples (see FIGS. 7A-7B).

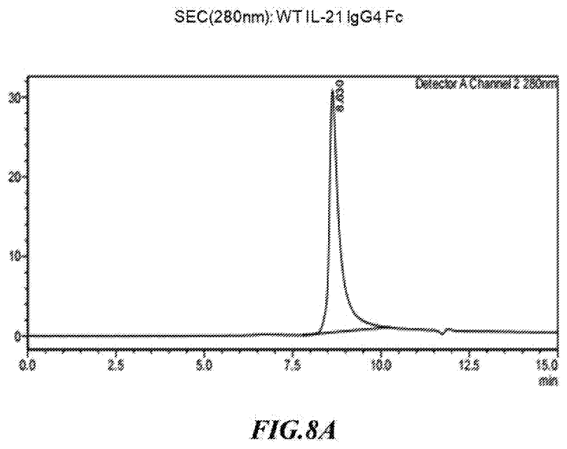

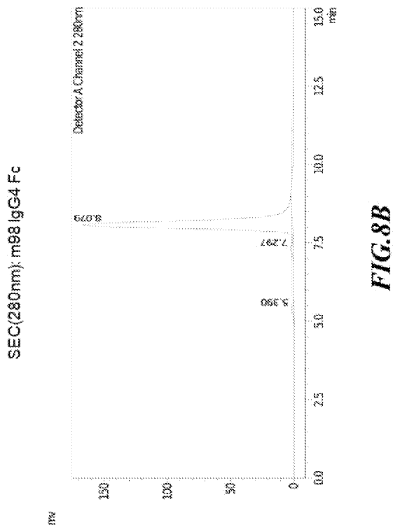

The purity of WT IL-21 IgG4 Fc and m98-IgG4 Fc proteins were further detected by size exclusion chromatography (SEC). SEC results at 280 nm showed that the WT IL-21 IgG4 Fc peaked at 8.6 min and m98-IgG4 Fc peaked at 8 min. The data indicate that these proteins were monomeric protein without aggregation (see FIGS. 8A-8B).

TABLE 7
Yield of the purified WT IL-21-IgG4 Fc and m98-IgG4 Fc

| Name | Substitutions | Expression (mg/L) |
|---|---|---|
| WT IL21-IgG4 Fc | NA | 73.4 |
| m98-IgG4 Fc | S70L, K72S, K73F, P79E | 460.4 |

### Alternative Preparation 2

The WT IL-21 or IL-21 variants of the disclosure can also be prepared and purified using other well-known tags, such as His tag.

The IL-21 variants were designed to be C-terminally linked with a GGGGS linker (SEQ ID NO: 91) and a His6-tag (SEQ ID NO: 81).

An exemplary sequence of IL-21-His tag is:

QGQDRHMIRMRQLIDIVDQLKNYVNDLVPEFLPAPEDVETNCEWS
AFSCFQKAQLKSANTGNNERIINVSIKKLKRKPPSTNAGRRQKHR
LTCPSCDSYEKKPPKEFLERFKSLLQKMIHQHLSSRTHGSEDSGG
GGSHHHHHH

(SEQ ID NO: 67; WT IL-21-His, wherein the underlined part represents the IL-21 sequence; the italic part represents G₄S linker (SEQ ID NO: 91) and His6-tag (SEQ ID NO: 81))

### 1) Construction of Expression Plasmid.

Based on the amino acid sequence of wildtype IL-21-His and IL-21 variant-His (including GGGGS linker (SEQ ID NO: 91) and His-tag), the DNA sequence was synthesized and confirmed by gene sequencing. The confirmed DNA sequence was constructed into the expression vector pcDNA3.1 (Thermo Fisher, catalog no. V79020). The plasmid containing the IL-21-His gene was transformed into E. coli DH5α cells. A large amount of both wildtype and variant plasmids was obtained by culturing the E. coli DH5α cells for amplification and plasmid purification.

### 2) Expression of Wildtype IL-21-his and IL-21 Variant-his in HEK293F Cells.

Cells were inoculated in 300 mL of media (Gibco™ FreeStyle™ 293 expression medium, catalog no. 12338018) in a 1-L shake flask at an inoculation volume of 0.5×10⁶ cells/ml. The cells were incubated for 24 hr in a shaker incubator at 37° C., 120 rpm, and 5% carbon dioxide concentration, until the cell density reaches 1×10⁶ cells/ml. Then, 300 μg of wildtype or mutant expression plasmid was added to 30 ml of PBS. The mixture was vortexed for 3s to thoroughly mix. About 1.2 ml (0.5 mg/ml) of transfection reagent PEI MAX 40K (Polysciences, catalog no. 24765) was added to the PBS/plasmid mixture. The mixture was held static for 20 min, and then added to the HEK293F cells. After transfection, the cells were incubated for 48-54 hr in a shaker incubator at 37° C., 120 rpm, and 5% carbon dioxide concentration. After incubation, the supernatant of the culture medium was isolated by centrifugation and prepared for protein purification.

### 3) Purification and Detection of Wildtype IL-21-his and IL-21 Variant-his.

The wildtype IL-21 and IL-21 variants containing a His-tag were purified using Ni-NTA agarose microspheres (Thermo Fisher, catalog no. R901). An appropriate amount ofNi-NTA agarose microspheres were added to the supernatant in the previous step, and incubated at 4° C. for 30 min. The wildtype IL-21 and IL-21 variants that were attached to the Ni-NTA agarose microspheres were collected by centrifuging the mixture with a low speed (1000 rpm) for 3 minutes. An appropriate volume of wash buffer (50 mM PBS, pH 7.4, 10 mMi imidazole) was then added to remove non-specific proteins. Finally, an appropriate volume of elution buffer (50 mM PBS, pH 7.4, 250 mM imidazole) was added to elute the wildtype or variants off the microspheres, thus the purified wildtype or variants were obtained.

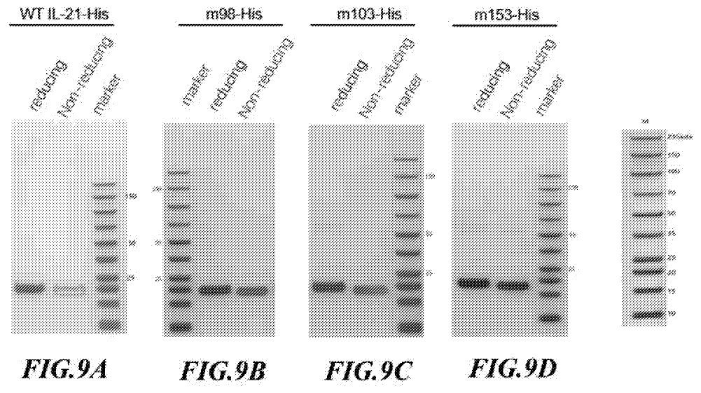

Wildtype IL-21 and some IL-21 variants designed in TABLE 1 were prepared in IL-21-His form. The purified wildtype IL-21-His and IL-21 variant-His proteins were detected by SDS-PAGE protein gel. SDS-PAGE protein gel detection results showed that the sizes of WT IL-21-His, m98-his, m103-his and m153-his were consistent with the theoretical molecular weight, for both their reducing samples and non-reducing samples (see FIGS. 9A-9D).

### Example 2: Recombinant Expression and Preparation of Mutant IL-21-Antibody Fusion Proteins

The IL-21 or IL-21 variants are shown in TABLE 1 or TABLE 2. The IL-21 or IL-21 variants and an antibody (e.g., anti PD-1 antibody) can be fused. Specifically, the IL-21 or IL-21 variants can be conjugated to the N-terminus and/or C-terminus of the heavy and/or light chain of the antibody.

### 1) Construction of Expression Plasmid.

Based on the amino acid sequence of wildtype IL-21 and IL-21 variants, the DNA sequence was synthesized and was designed to include the sequences of the heavy or light chain of the antibody. The sequences were confirmed by gene sequencing. The confirmed DNA sequence was constructed into the expression vector pcDNA3.1 (Thermo Fisher, catalog no. V79020). The plasmid containing the IL-21 gene was transformed into E. coli DH5α cells. A large amount of both wildtype-antibody and variants-antibody plasmids were obtained by culturing the E. coli DH5α cells for amplification and plasmid purification.

### 2) Expression of Wildtype IL-21-Antibody and IL-21 Variants-Antibody Fusion Proteins in HEK293F Cells.

Cells were inoculated in 300 mL of media (Gibco™ FreeStyle™ 293 expression medium, catalog no. 12338018) in a 1-L shake flask at an inoculation volume of 0.5×10⁶ cells/ml. The cells were incubated for 24 hr in a shaker incubator at 37° C., 120 rpm, and 5% carbon dioxide concentration, until the cell density reach 1×10⁶ cells/ml. Then, 300 μg of wildtype-antibody or variants—antibody expression plasmid was added to 30 ml of PBS. The mixture was vortexed for 3s to thoroughly mix. About 1.2 ml (0.5 mg/ml) of transfection reagent PEI MAX 40K (Polysciences, catalog no. 24765) was added to the PBS/plasmid mixture. The mixture was held static for 20 min, and then was added to the HEK293F cells. After transfection, the cells were incubated for 48-54 hr in a shaker incubator at 37° C., 120 rpm, and 5% carbon dioxide concentration. After incubation, the supernatant of the culture medium was isolated by centrifugation and prepared for protein purification.

### 3) Purification of Wildtype IL-21-Antibody and IL-21 Variants-Antibody.

The wildtype IL-21-antibody and IL-21 variants-antibody fusion proteins were purified using Protein A agarose microspheres. An appropriate amount of Protein A agarose microspheres were added to the supernatant in the previous step, and incubated at 4° C. for 10 min. The wildtype IL-21-antibody and IL-21 variants-antibody fusion proteins that were attached to the Protein A agarose microspheres were collected by centrifuging the mixture with a low speed (1000 rpm) for 3 minutes. An appropriate volume of wash buffer (50 mM PBS, pH 7.4) was then added to remove non-specific proteins. Finally, an appropriate volume of elution buffer (Glycine-HCl, pH3) was added to elute the wildtype-antibody or variants-antibody fusion proteins off the microspheres, thus the purified wildtype-antibody or variants-antibody fusion proteins were obtained.

PD-1 antibody-IL-21 fusion proteins were constructed so that IL-21 was attached to the C-terminal of the heavy chain of a mouse PD-1 antibody via a linker (GGGGS (SEQ ID NO: 91)). The fusion proteins (anti-mPD1 antibody_WT IL21 fusion protein, anti-mPD1 antibody-IL21m98 fusion protein, anti-mPD1 antibody-IL21m100 fusion protein and anti-mPD1 antibody-IL21m153 fusion protein) were prepared using the wildtype (WT IL21) and IL21 mutants (m98, m100, and m153) and the following anti-mPD1 antibody sequences:

Anti-mPD1 antibody heavy chain sequence:
(SEQ ID NO: 92)
EVQLQESGPGLVKPSQSLSLTCSVTGYSITSSYRWNWIRKFPGNR
LEWMGYINSAGISNYNPSLKRRISITRDTSKNQFFLQVNSVTTED
AATYYCARSDNMGTTPFTYWGQGTLVTVSSAKTTPPSVYPLAPGS
AAQTNSMVTLGCLVKGYFPEPVTVTWNSGSLSSGVHTFPAVLQSD
LYTLSSSVTVPSSTWPSETVTCNVAHPASSTKVDKKIVPRDCGCK
PCICTVPEVSSVFIFPPKPKDVLTITLTPKVTCVVVAISKDDPEV
QFSWFVDDVEVHTAQTQPREEQFNSTFRSVSELPIMHQDWLNGKE
FKCRVNSAAFPAPIEKTISKTKGRPKAPQVYTIPPPKEQMAKDKV
SLTCMITDFFPEDITVEWQWNGQPAENYKNTQPIMDTDGSYFVYS
KLNVQKSNWEAGNTFTCSVLHEGLHNHHTEKSLSHSPG
Anti-mPD1 antibody light chain sequence:
(SEQ ID NO: 93)
DIVMTQGTLPNPVPSGESVSITCRSSKSLLYSDGKTYLNWYLQRP
GQSPQLLIYWMSTRASGVSDRFSGSGSGTDFTLKISGVEAEDVGI
YYCQQGLEFPTFGGGTKLELKRADAAPTVSIFPPSSEQLTSGGAS
VVCFLNNFYPKDINVKWKIDGSERQNGVLNSWTDQDSKDSTYSMS
STLTLTKDEYERHNSYTCEATHKTSTSPIVKSFNRNEC

Of the IL-21_PD-1 antibody fusion proteins thus prepared, anti-mPD1 antibody-IL21m98 fusion protein is taken as an example to illustrate the full-length sequence of the anti-mPD-1 heavy chain fused with IL-21:

(SEQ ID NO: 94)
EVQLQESGPGLVKPSQSLSLTCSVTGYSITSSYRWNWIRKFPGNR
LEWMGYINSAGISNYNPSLKRRISITRDTSKNQFFLQVNSVTTED
AATYYCARSDNMGTTPFTYWGQGTLVTVSSAKTTPPSVYPLAPGS
AAQTNSMVTLGCLVKGYFPEPVTVTWNSGSLSSGVHTFPAVLQSD
LYTLSSSVTVPSSTWPSETVTCNVAHPASSTKVDKKIVPRDCGCK
PCICTVPEVSSVFIFPPKPKDVLTITLTPKVTCVVVAISKDDPEV
QFSWFVDDVEVHTAQTQPREEQFNSTFRSVSELPIMHQDWLNGKE
FKCRVNSAAFPAPIEKTISKTKGRPKAPQVYTIPPPKEQMAKDKV
SLTCMITDFFPEDITVEWQWNGQPAENYKNTQPIMDTDGSYFVYS
KLNVQKSNWEAGNTFTCSVLHEGLHNHHTEKSLSHSPGGGGGSQG
QDRHMIRMRQLIDIVDQLKNYVNDLVPEFLPAPEDVETNCEWSAF
SCFQKAQLKSANTGNNERIINVLISYLKRKPESTNAGRRQKHRLT
CPSCDSYEKKPPKEFLERFKSLLQKMIHQHLSSRTHGSEDS

### (the Underlined Segment Represents the Mature IL-21 Sequence; the Italic Part Represents Flexible Linker; and the Rest is Anti-mPD1 Ab)

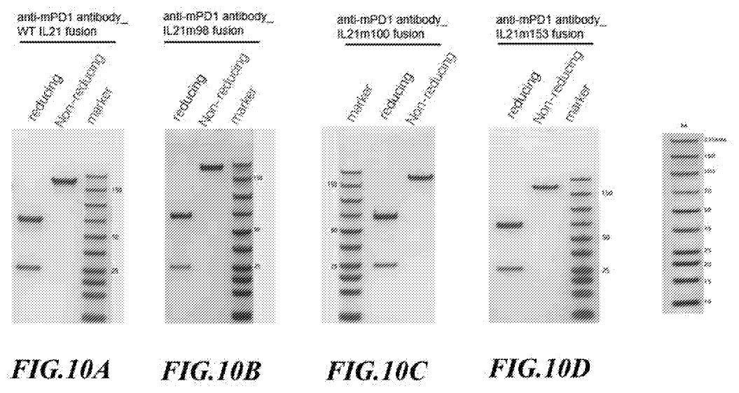

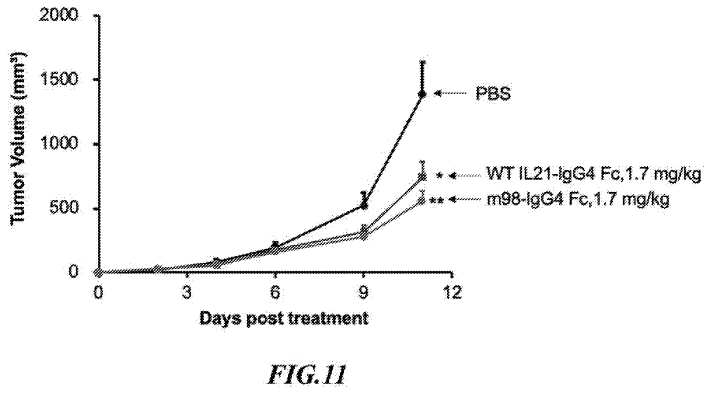

The purified fusion proteins (anti-mPD1 antibody IL21 fusion protein, anti-mPD1 antibody-IL21m98 fusion protein, anti-mPD1 antibody-IL21m100 fusion protein and anti-mPD1 antibody-IL21m153 fusion protein) were detected by SDS-PAGE protein gel. SDS-PAGE protein gel detection results showed that the sizes of fusions were consistent with the theoretical molecular weight, for both their reducing samples and non-reducing samples (see FIGS. 10A-10D).

### Example 3: Determination of Thermal Stability of IL-21 Mutant Proteins

To assess conformational stability, the melting point Tm (° C.) values, which indicated the structural stability of the samples, were obtained by monitoring the intrinsic tryptophan and tyrosine fluorescence at the emission wavelengths of 330 nm and 350 nm.

IL-21 mutant protein stability was assessed by detecting small changes in tryptophan and tyrosine fluorescence in the mutant IL-21 protein using the label-free nanoDSF technique (Prometheus, PR NT.48). Briefly, Wildtype IL-21 or mutant IL-21 proteins were diluted in PBS to 0.4 mg/ml. The capillaries were filled with 10 μL of Wildtype IL-21 or mutant IL-21 proteins, and then placed on the sample holder. A temperature gradient of 1° C./min from 25 to 95° C. was applied. and the intrinsic protein fluorescence at 330 and 350 nm was recorded. The data were analyzed using the data analysis software provided by NT.48 instrument.

As shown in Table 8: m98-His and m153-His demonstrated high thermal stability with Tm of 62.02° C. and 63.12° C. respectively, which were notably higher than that of WT IL-21-His (Tm 48.47° C.).

TABLE 8
The melting temperatures (Tm) of wildtype IL-21 and mutant
IL-21 were determined by the label-free nanoDSF technique.

| | WT IL-21-His | m98-His | m153-His |
|---|---|---|---|
| Tm(° C.) | 48.47 | 62.02 | 63.12 |

### Example 4: Determination of the Affinity of Mutant IL-21 Protein to Human IL-21R

The affinity of mutant IL-21 to human IL-21R (ACRO, Catalog: ILR-H5226) was determined by label-free biolayer interferometry (Octet® R8, BLI) as follows:

1: Wildtype and Mutant IL-21-Fc proteins were prepared and diluted with buffer (10 mM PBS+0.02% TW-20+0.1% BSA, pH 7.4) to a final concentration of 5 μg/ml.

2: The IL-21R protein was diluted with buffer (10 mM PBS+0.02% tween-20+0.1% BSA, pH 7.4) from the initial concentration of 1000 nM to 3.9 nM in a 4-fold gradient to yield a total of 5 concentrations: 1000 nM, 250 nM, 62.5 nM, 15.6 nM, and 3.9 nM, respectively.

3: The Protein A sensor was soaked in buffer (10 mM PBS+0.02% tween-20+0.1% BSA, pH 7.4) for 10 min to equilibrate.

4: The IL-21 mutant-Fc protein was loaded onto the Protein A sensor. The loading time was 90s. The mutant-Fc protein was then eluted and then equilibrated for 30 s. The mutant-Fe protein was then combined with 4 different concentrations of IL-21R protein. The binding time was 120 s. Finally, the bound IL-21R protein was eluted with a buffer. The dissociation time was 180 s.

5: The Octet Data Acquisition Software of Octet® R8 was used for data processing and data fitting.

The affinity data in TABLE 4 show that the affinity (K_{D}) of WT IL-21-Fc to IL-21R was 4.57E-10, and the affinity of IL-21 mutant-Fc to IL-21R was lower than that of the wildtype IL-21-Fc to IL-21R.

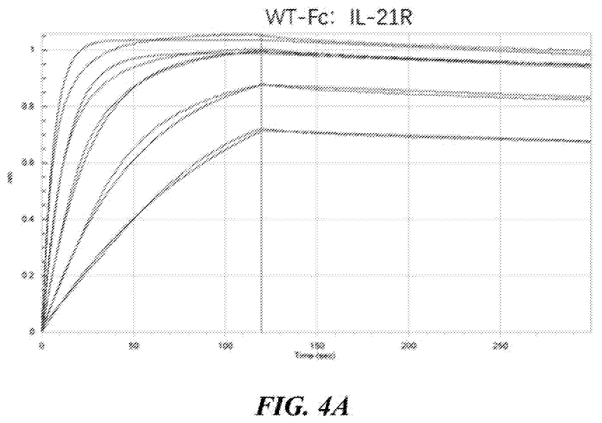

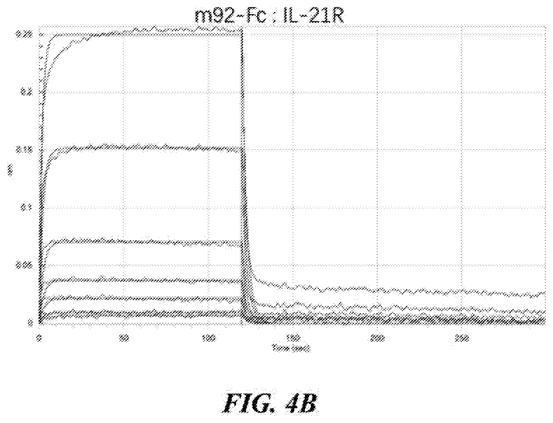

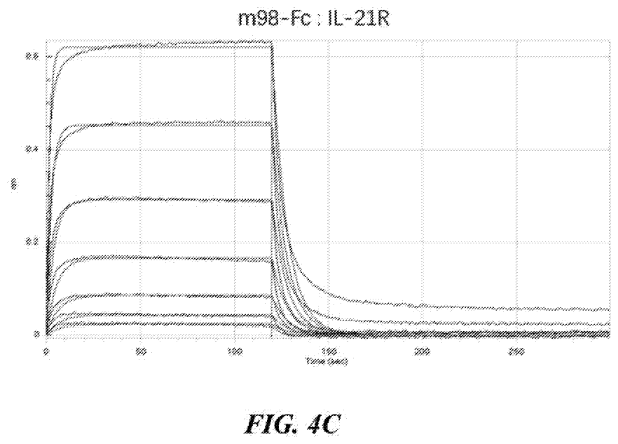

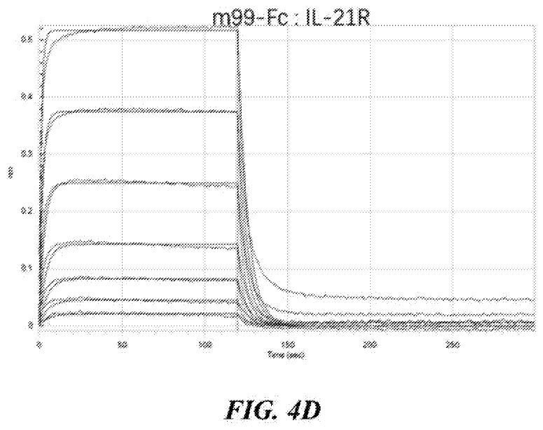

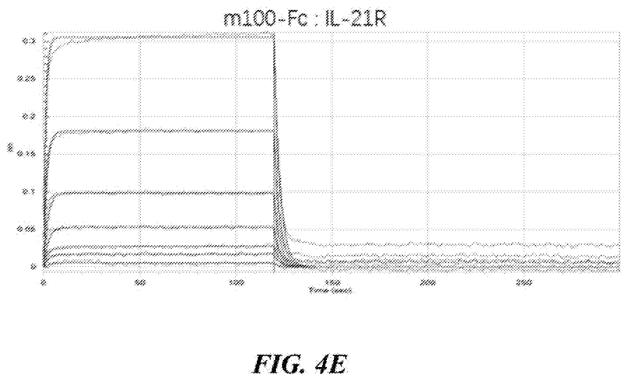

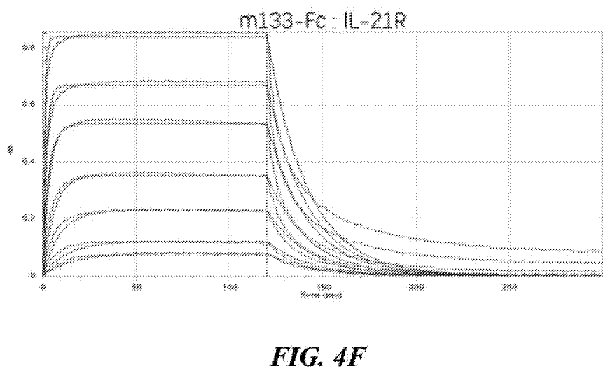

The interaction of WT IL-21-Fc with IL-21R was “fast Kon, slow Koff.” However, the interaction of mutants (such as m92-Fc, m98-Fc, m99-Fc, m100-Fc and m133-Fc) with IL-21R was fast Kon, fast Koff (see FIGS. 4A-4F).

TABLE 4
Affinity between IL-21-Fc (wildtype or mutants) and IL-21R

| | Affinity between IL-21-Fc and IL-21R (BLI) | | | | | |
|---|---|---|---|---|---|---|
| | KD (M) | Kon(1/Ms) | Koff(1/s) | RMax | Full X\hat{ }2 | Full R\hat{ }2 |
| WT IL-21-Fc | 4.57E−10 | 6.31E+05 | 2.88E−04 | 1.0369 | 0.4155 | 0.9943 |
| m103-Fc | 5.08E−09 | 6.99E+05 | 3.55E−03 | 1.0218 | 1.2023 | 0.9783 |
| m131-Fc | 6.50E−09 | 7.30E+05 | 4.74E−03 | 0.9819 | 0.6393 | 0.9893 |
| m99-Fc | 5.70E−07 | | | | | |
| m133-Fc | 2.30E−07 | | | | | |
| m100-Fc | 2.20E−06 | | | | | |
| m72-Fc | very weak | | | | | |
| m74-Fc | 1.00E−06 | | | | | |
| m76-Fc | 1.41E−08 | 7.68E+04 | 1.08E−03 | 0.3799 | 0.2857 | 0.9852 |
| m94-Fc | very weak | | | | | |
| m102-Fc | very weak | | | | | |
| m111-Fc | very weak | | | | | |
| m137-Fc | very weak | | | | | |
| m138-Fc | very weak | | | | | |
| m88-Fc | 3.23E−08 | 4.67E+05 | 1.51E−02 | 0.7207 | 3.2364 | 0.9478 |
| m98-Fc | 6.40E−07 | | | | | |
| m89-Fc | very weak | | | | | |
| m95-Fc | very weak | | | | | |
| m96-Fc | very weak | | | | | |
| m97-Fc | very weak | | | | | |
| m92-Fc | 2.90E−06 | | | | | |
| m127-Fc | very weak | | | | | |
| m128-Fc | 1.50E−06 | | | | | |
| m86-Fc | very weak | | | | | |
| m87-Fc | very weak | | | | | |
| m129-Fc | very weak | | | | | |
| m109-Fc | very weak | | | | | |
| m110-Fc | very weak | | | | | |
| m121-Fc | very weak | | | | | |
| m29-Fc | very weak | | | | | |
| m30-Fc | very weak | | | | | |
| m31-Fc | very weak | | | | | |
| m32-Fc | very weak | | | | | |

Note:
“very weak” in TABLE 4 indicates that the detection data could not be fitted since the affinity was too low to be detected.

### Example 5: Determination of p-STAT3 Phosphorylation in HuT78 Cells by IL-21 Mutants

IL-21R is expressed on the surface of HuT78 cells. IL-21 is capable of binding to IL-21R, thereby stimulating the phosphorylation of Tyr705 of STAT3 protein, a downstream signaling protein of IL-21R in HuT78 cells. The phosphorylation effect of phosphorylation of Tyr705 of STAT3 protein was measured utilizing phospho-STAT3 (Tyr705) kits/62AT3PET (Cisbio), which served as a proxy to evaluate the ability of IL-21 mutants to activate the downstream signaling pathway of IL-21R. STAT3 phosphorylation was detected using phosphor-STAT3 (Tyr705) assay kit accordingly to manufacturer's recommendation (Cisbio, Catalog: 64NT3PEH).

### STAT3 Phosphorylation Assays

1) The IL-21-Fc fusion protein was diluted with IMDM Complete Medium from the initial concentration of 1000 nM to 0.0001 nM in a 10-fold gradient to yield a total of 8 concentrations: 1000 nM, 100 nM, 10 nM, 1 nM, 0.1 nM, 0.0 nM, 0.001 nM and 0.0001 nM, respectively.

2) HuT78 cells were seeded at a density of 33,000 cells per well (8 μl) in HTRF 96-well low volume white plate (Cisbio, catalog no. 66PL96100), HuT78 cells were then stimulated with 12 uL/well of IL-21-Fc fusion at 37° C. for 30 minutes. and then added 4 μl supplemented lysis buffer(4X) (Cisbio, catalog no. 62AT3PET) per well and incubated 30 min at room temperature under shaking. Finally, added 4 μl Premix antibody solutions (Cisbio, catalog no. 62AT3PET) and incubated overnight at room temperature.

3) FRET signal from the assay was detected using EnVision Multilable Plate Reader (Perkin Elmer). Data were analyzed by first determining the HTRF ratio as recommended by Cisbio and then calculating fold over background values using data from unstimulated cells. EC50 values were calculated by fitting dependent data to the four-parameter logistic model using dotmatics software.

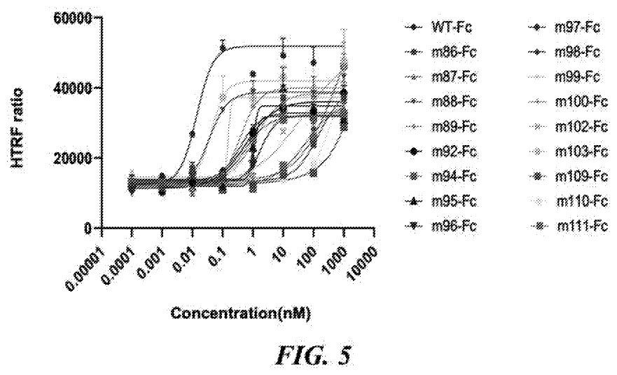

The experimental results showed that the EC₅₀ of WT IL-21-Fc to activate the phosphorylation of STAT3 in HuT78 cells was 0.01412 nM. Since the affinity of the IL-21 mutant-Fc to IL-21R protein was reduced to varying degrees, the EC₅₀ values of IL-21 mutant-Fc to activate STAT3 phosphorylation in HuT78 cells were all greater. (see FIG. 5 and TABLE 5).

TABLE 5
STAT3 phosphorylation in HuT78 cells by
wildtype IL-21-Fc or IL-21 mutant-Fc

| | pSTAT3 activation(EC50, nM) |
|---|---|
| WT IL-21-Fc | 0.01412 |
| m103-Fc | 0.03936 |
| m99-Fc | 0.2886 |
| m100-Fc | 1.158 |
| m94-Fc | 0.5697 |
| m102-Fc | 13.35 |
| m111-Fc | 219.7 |
| m88-Fc | 0.03179 |
| m98-Fc | 0.3407 |
| m89-Fc | 0.3986 |
| m95-Fc | 1.013 |
| m96-Fc | 2.125 |
| m97-Fc | 1.291 |
| m92-Fc | 0.567 |
| m86-Fc | 163.8 |
| m87-Fc | 47.68 |
| m109-Fc | 792.1 |
| m110-Fc | 840.4 |

### Example 6: Evaluating Anti-Tumor Activity of IL-21-Antibody Fusion Proteins In Vivo

In vivo efficacy of the IL-21-antibody fusion proteins prepared in Example 2 (anti-mPD1 antibody-IL21m98 fusion protein, anti-mPD1 antibody-IL21m100 fusion protein and anti-mPD1 antibody-IL21m153 fusion protein) were further evaluated and compared with anti-mPD1 antibody_WT IL21 fusion protein.

The hIL21R KI mouse model was established, and the tumor-bearing mice were divided into control group and experimental group. The control group received vehicle, and the experimental group was further into (1) WT IL21_antibody fusion proteins; (2) antibodies alone; and (3) IL21 mutant_antibody fusion proteins. In each of the three subgroups, various concentrations and/or dosing regimens were optimized. In order to monitor the tumor burden over time without sacrificing the animals with ease, subcutaneous tumors were preferred. The following parameters for efficacy and safety were measured at multiple time points before and after injection of the IL21 mutants_antibody conjugates: percent test/control (% T/C) tumor weights calculated on each day that tumors were measured, tumor growth delay, net log cell kill, median days to a defined tumor weight or to a specified number of tumor doublings, tumor regression, animal survival rate, and basic health conditions (e.g., weight and locomotion). For tumors that were not subcutaneous, tumors were resected and physically weighed, then the same criteria were used for determining tumor borders. Alternatively, tumors could be monitored with imaging technologies (e.g., bioluminescence, ultrasound, magnetic resonance imaging).

Specifically, MC38 murine colon cancer cells (1×10⁶ cells) were implanted subcutaneously into the flanks of C57BL/6-Il21r^{tm1(tL21R)}/Bcgen mice (Beijing Biocytogen Co., Ltd, stock No.: 110766). Mice bearing MC38 cells were treated with either PBS, 3 mg/kg anti-mPD1 antibody, 3.65 mg/kg anti-mPD1 antibody_WT IL21 fusion protein, 3.65 mg/kg anti-mPD1 antibody_IL21m98 fusion protein, 3.65 mg/kg anti-mPD1 antibody_IL21m100 fusion protein, or 3.65 mg/kg anti-mPD1 antibody_IL21m153 fusion protein twice per week for 2 weeks (4 total doses). Body weight and tumor volume were measured every 3 or 4 days throughout the study. Tumor measurements (length (L) and width (W)) were collected three times per week using digital calipers, and the tumor volume was calculated (LxWxW)/2. Results are shown in Table 6 below.

TABLE 6
Tumor growth inhibition

| | | anti-mPD1 | | | |
|---|---|---|---|---|---|
| | | antibody_WT | anti-mPD1 | anti-mPD1 | anti-mPD1 |
| | anti-mPD1 | IL21 fusion | antibody_IL21m98 | antibody_IL21m100 | antibody_IL21m153 |
| | antibody | protein | fusion protein | fusion protein | fusion protein |
| TGI (tumor | 56.91% | 16.51% | 96.90% | 91.46% | 93.40% |
| growth | | | | | |
| inhibition) | | | | | |

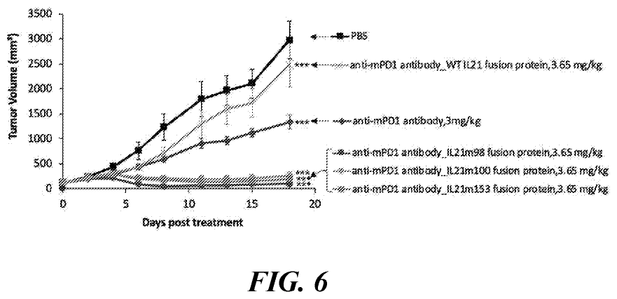

FIG. 6 illustrates tumor growth inhibition (error bars, SEM) in MC38 tumor-bearing mice upon treatment with anti-mPD1 antibody_IL21m fusion proteins and anti-mPD1 antibody_WT IL21 fusion protein at the indicated concentrations. n=8 mice/group. Two-way ANOVA with Tukey multiple comparison was performed, endpoint analyses are shown; ns, not significant; *, P<0.05; **, P<0.01; ***, P<0.001; n=8 mice/group.

As shown in FIG. 6 and Table 6, anti-mPD1 antibody_IL21m fusion proteins (anti-mPD1 antibody_1L21m98 fusion protein, anti-mPD1 antibody_IL21m100 fusion protein and anti-mPD1 antibody_IL21m153 fusion protein) significantly reduced tumor growth in MC38 tumors compared to anti-mPD1 antibody as well as anti-mPD1 antibody_WT IL21 fusion protein.

### Example 7: Evaluating Anti-Tumor Activity of IL-21 Proteins In Vivo

The purified WT IL21-IgG4 Fc and exemplary m98-IgG4 Fc proteins were used in an in vivo study. The full-length amino acid sequence of the matured m98-IgG4 Fc was shown below:

QGQDRHMIRMRQLIDIVDQLKNYVNDLVPEFLPAPEDVETNCEWS
AFSCFQKAQLKSANTGNNERIINVLISFLKRKPESTNAGRRQKHR
LTCPSCDSYEKKPPKEFLERFKSLLQKMIHQHLSSRTHGSEDSES
KYGPPCPSCPAPEFLGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQ
EEMTKNQVSLTCLVKGFYPSDIAVEWESNGQPENNYKTTPPVLDS
DGSFFLYSRLTVDKSRWQEGNVFSCSVMHEALHNHYTQKSLSLSL
GK

(Amino acid sequence of m98-IgG4 Fc construct (SEQ ID NO. 70), wherein the underlined segment represents the mature m98 sequence; the italic part represents human IgG4 Fc-tag). In other word, SEQ ID NO. 70 did not comprise the 18 aa signal peptide.

B16-F10 melanoma cancer cells (5×10⁵ cells) were implanted subcutaneously into the flanks of C57BL/6-Il21T^{tm1(IL21R)}/Bcgen mice (Beijing Biocytogen Co., Ltd, stock No.: 110766). At around the 2^{nd} day post-inoculation, the tumor-bearing mice were divided into 3 groups with 10 mice per group by random block method. Mice bearing B16-F10 cells were treated with either PBS, 1.7 mg/kg WT IL21-IgG4 Fc protein or 1.7 mg/kg m98-IgG4 Fc protein Once every three days for 11 days (4 total doses). Body weight and tumor volume were measured every 2 or 3 days throughout the study. Tumor measurements (length (L) and width (W)) were collected three times per week using digital calipers, and the tumor volume was calculated (LxWxW)/2.

TABLE 9
Tumor growth inhibition

| | WT IL21-IgG4 Fc | m98-IgG4 Fc |
|---|---|---|
| | (1.7 mg/kg) | (1.7 mg/kg) |
| TGI (tumor | 46.39% | 59.93% |
| growth | | |
| inhibition) | | |

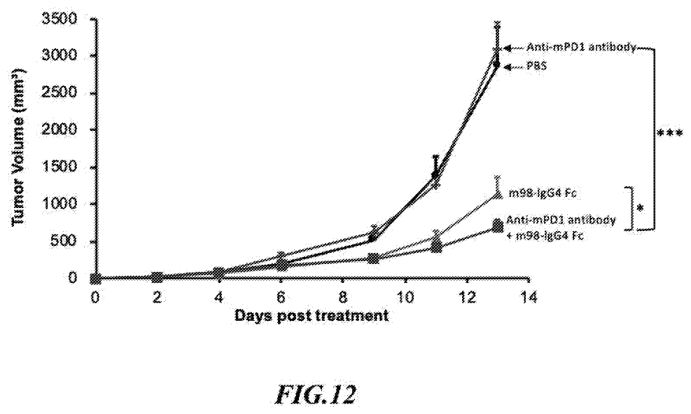

FIG. 11 illustrates tumor growth inhibition (error bars, SEM) in B16-F 10 tumor-bearing mice upon treatment with WT IL21-IgG4 Fc protein and m98-IgG4 Fc protein at the indicated concentrations. n=10 mice/group. Two-way ANOVA with Tukey multiple comparison was performed, endpoint analyses are shown; ns, not significant; *, P<0.05; **, P<0.01; ***, P<0.001; n=10 mice/group.

As shown in FIG. 11 and Table 9, m98-IgG4 Fc protein significantly further reduced tumor growth in B16-F10 tumors compared to WT IL21-IgG4 Fc protein. The in vivo studies showed the favorable tumor inhibition effect of the muteins of the present disclosure.

### Example 8: Evaluating Anti-Tumor Activity of IL-21 Protein In Vivo

B16-F10 melanoma cancer cells (5×10⁵ cells) were implanted subcutaneously into the flanks of C57BL/6-1121r ml(I^{L2}1R)/Bcgen mice (Beijing Biocytogen Co., Ltd, stock No.: 110766). At around the 2^{nd} a day post-inoculation, The tumor-bearing mice were divided into 4 groups with 10 mice per group by random block method. Each group of mice bearing B16-F10 cells were treated with either PBS, 1.7 mg/kg m98-IgG4 Fc protein, 3 mg/kg anti-mPD1 antibody or 1.7 mg/kg m98-IgG4 Fc combined with 3 mg/kg anti-mPD1 antibody, Once every three days for 13 days (on Day 0, 3, 6, 9 and 12, 5 total doses). Body weight and tumor volume were measured every 2 or 3 days throughout the study. Tumor measurements (length (L) and width (W)) were collected three times per week using digital calipers, and the tumor volume was calculated (LxWxW)/2.

TABLE 10
Tumor growth inhibition

| | anti-mPD1 | | anti-mPD1 antibody |
|---|---|---|---|
| | antibody | m98-IgG4 Fc | (3 mg/kg) + |
| | (3 mg/kg) | (1.7 mg/kg) | m98-IgG4 Fc (1.7 mg/kg) |
| TGI (tumor | −8.35% | 60.03% | 75.67% |
| growth | | | |
| inhibition) | | | |

FIG. 12 illustrates tumor growth curves (error bars, SEM) in B16-F10 tumor-bearing mice upon treatment with m98-IgG4 Fc protein, anti-mPD1 antibody and m98-IgG4 Fc combined with anti-mPD1 antibody at the indicated concentrations. n=10 mice/group. Two-way ANOVA with Tukey multiple comparison was performed, endpoint analyses are shown; ns, not significant; *, P<0.05; **, P<0.01; ***, P<0.001; n=10 mice/group.

As shown in FIG. 12 and Table 10, administration of 3 mg/kg anti-mPD1 antibody as a single agent exhibited no anti-tumor activity, and administration of 1.7 mg/kg m98-IgG4 Fc as a single agent resulted in partial anti-tumor activity. However, 3 mg/kg anti-mPD1 antibody in combination with 1.7 mg/kg m98-IgG4 Fc resulted in statistically significant and synergistic anti-tumor activity as compared to treatment with anti-mPD1 antibody (P<0.001) or 1.7 mg/kg m98-IgG4 Fc alone (P<0.05).

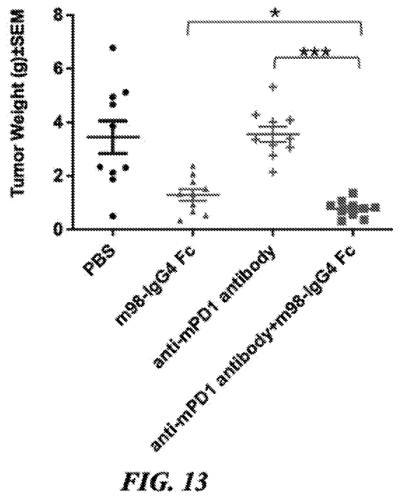

FIG. 13 illustrates tumor weights (error bars, SEM) in B16-F10 tumor-bearing mice upon treatment with m98-IgG4 Fc protein, anti-mPD1 antibody and m98-IgG4 Fc combined with anti-mPD1 antibody at the indicated concentrations. n=10 mice/group. Two-way ANOVA with Tukey multiple comparison was performed, endpoint analyses are shown; ns, not significant; *, P<0.05; **, P<0.01; ***, P<0.001; n=10 mice/group.

As shown in FIG. 13, the average tumor masses of anti-mPD1 antibody-treated mice (P<0.001) and 1.7 mg/kg m98-IgG4 Fe-treated mice (P<0.05) were significantly greater than those of combination treated mice. No abnormal body weight changes or signs of toxicity were observed. These data suggested that m98-IgG4 Fc and combination administration significantly reduced tumor growth in B16-F10 model.

The in vivo studies showed the favorable tumor inhibition effect of the muteins of the present disclosure, indicating their therapeutic potential as a therapeutic agent as single agent, in combination or used in multi-functional fusion molecules.

What is claimed is:

1. A polypeptide comprising an interleukin-21 (IL-21) variant with an amino acid residue substitution at position P79 corresponding to a wildtype human IL-21.

2. The polypeptide of claim 1, wherein the wildtype human IL-21 comprises a sequence as set forth in SEQ ID NO: 1 or 2.

3. The polypeptide of claim 1 or 2, wherein the amino acid residue substitution at position P79 is P79E or P79C.

4. The polypeptide of claim 3, wherein the amino acid residue substitution at position P79 is P79E.

5. The polypeptide of claim 3, wherein the amino acid residue substitution at position P79 is P79C, and wherein the polypeptide further comprises one or more amino acid residue substitutions with cysteine in the region of positions 1-10 corresponding to the wildtype human IL-21.

6. The polypeptide of claim 5, wherein the one or more amino acid residue substitutions are selected from the group consisting of R5C, H6C and R9C.

7. The polypeptide of any one of claims 5-6, wherein the cysteine residues of the one or more amino acid residue substitutions in the region of positions 1-10 form a disulfide linkage together with the cysteine residue at position P79.

8. The polypeptide of any one of claims 1-7, wherein the polypeptide comprises one or more amino acid residue substitutions at positions selected from the group consisting of R5, H6, R9, Q12, L13, D15, I16, S70, K72, K73, R76, and P78.

9. The polypeptide of claim 8, wherein the polypeptide comprises one or more amino acid residue substitutions at positions selected from the group consisting of S70, K72, K73, and R76.

10. The polypeptide of claim 8, wherein the polypeptide further comprises an amino acid residue substitution at position K73 corresponding to the wildtype human IL-21.

11. The polypeptide of claim 10, wherein the amino acid residue substitution at position K73 is an aromatic amino acid.

12. The polypeptide of claim 10, wherein the amino acid residue substitution at position K73 is K73Y or K73F.

13. The polypeptide of any one of claims 8-12, wherein the polypeptide further comprises an amino acid residue substitution at position S70 corresponding to the wildtype human IL-21.

14. The polypeptide of claim 13, wherein the amino acid residue substitution at S70 is S70L or S70A.

15. The polypeptide of any one of claims 8-14, wherein the polypeptide further comprises an amino acid residue substitution at position R76 corresponding to the wildtype human IL-21.

16. The polypeptide of claim 15, wherein the amino acid residue substitution at R76 is R76F.

17. The polypeptide of any one of claims 8-16, wherein the polypeptide further comprises an amino acid residue substitution at position K72 corresponding to the wildtype human IL-21.

18. The polypeptide of claim 17, wherein the amino acid residue substitution at K72 is a non-aromatic amino acid comprising a side chain hydroxyl, and optionally the amino acid residue substitution at K72 is K72S.

19. The polypeptide of claim 8, wherein the polypeptide further comprises an amino acid residue substitution at position Q12 corresponding to the wildtype human IL-21.

20. The polypeptide of claim 19, wherein the amino acid residue substitution at Q12 is Q12W.

21. The polypeptide of claim 8, wherein the polypeptide further comprises an amino acid residue substitution at position L13 corresponding to the wildtype human IL-21.

22. The polypeptide of claim 21, wherein the amino acid residue substitution at L13 is L13A.

23. The polypeptide of claim 8, wherein the polypeptide further comprises an amino acid residue substitution at position D15 corresponding to the wildtype human IL-21.

24. The polypeptide of claim 23, wherein the amino acid residue substitution at D15 is D15L, D15K or D15R.

25. The polypeptide of claim 8, wherein the polypeptide further comprises an amino acid residue substitution at position 116 corresponding to the wildtype human IL-21.

26. The polypeptide of claim 25, wherein the amino acid residue substitution at I16 is 116R or 116W.

27. The polypeptide of claim 8, wherein the polypeptide further comprises an amino acid residue substitution at position P78 corresponding to the wildtype human IL-21.

28. The polypeptide of claim 27, wherein the amino acid residue substitution at P78 is P78G or P78E.

29. A polypeptide comprising an interleukin-21 (IL-21) variant with an amino acid residue substitution at position S70 corresponding to a wildtype human IL-21.

30. The polypeptide of claim 29, wherein the wildtype human IL-21 comprises a sequence as set forth in comprises SEQ ID NO: 1 or 2.

31. The polypeptide of claim 29 or 30, wherein the amino acid residue substitution at position S70 is S70L or S70A.

32. A polypeptide comprising an interleukin-21 (IL-21) variant with an amino acid residue substitution at position K73 corresponding to a wildtype human IL-21.

33. The polypeptide of any one of claims 29-31, wherein the polypeptide further comprises an amino acid residue substitution at position K73 corresponding to the wildtype human IL-21.

34. The polypeptide of claim 32 or 33, wherein the polypeptide comprises an amino acid residue substitution with an aromatic amino acid at position K73 corresponding to the wildtype human IL-21.

35. The polypeptide of claim 34, wherein the amino acid residue substitution at position K73 is K73Y or K73F.

36. The polypeptide of any one of claims 32-35, wherein the polypeptide further comprises one, two, or three, or more amino acid residue substitutions at positions selected from the group consisting of: R5, H6, R9, Q12, L13, D15, I16, K72, R76, P78 and P79.

37. The polypeptide of any one of claims 32-35, wherein the polypeptide further comprises one, two, or three, or four amino acid residue substitutions at positions selected from the group consisting of Q12, D15, I16, and K72.

38. The polypeptide of claim 36, wherein the polypeptide comprises an amino acid residue substitution selected from the group consisting of: 116R and 116W.

39. The polypeptide of claim 36, wherein the polypeptide comprises an amino acid residue substitution selected from the group consisting of: D15L, D15K, and D15R.

40. The polypeptide of claim 36, wherein the polypeptide comprises an amino acid residue substitution selected from the group consisting of: K72S and Q12W.

41. The polypeptide of any one of claims 29-31, wherein the polypeptide further comprises an amino acid residue substitution at position R76 corresponding to the wildtype human IL-21.

42. The polypeptide of claim 41, wherein the amino acid residue substitution at position R76 is R76F.

43. The polypeptide of claim 41 or 42, wherein the polypeptide further comprises one, two, or three, or more amino acid residue substitutions at positions selected from the group consisting of: R5, H6, R9, Q12, L13, D15, I16, K72, K73, P78 and P79.

44. The polypeptide of claim 41 or 42, wherein the polypeptide further comprises one, two, or three, or four amino acid residue substitutions at positions selected from the group consisting of: Q12, D15, I16, and K72.

45. The polypeptide of claim 43, wherein the polypeptide comprises an amino acid residue substitution selected from the group consisting of: 116R and 116W.

46. The polypeptide of claim 43, wherein the polypeptide comprises an amino acid residue substitution selected from the group consisting of: D15L, D15K, and D15R.

47. The polypeptide of claim 43, wherein the polypeptide comprises an amino acid residue substitution selected from the group consisting of: K72S and Q12W.

48. The polypeptide of any one of the preceding claims, wherein the amino acid segment of STNAGRRQKHR (SEQ ID NO: 71) at positions 80-90 corresponding to the wildtype human IL-21 is substituted with a short peptide linker.

49. The polypeptide of claim 48, wherein the length of the short peptide linker is from 4 to 10 or from 5 to 9 amino acid residues.

50. The polypeptide of claims 48 or 49, wherein the short peptide linker comprises one or more Gly-Ser units, and optionally one glutamic acid residue.

51. The polypeptide of claim 50, wherein the short peptide linker comprises the glutamic acid residue, and the glutamic acid residue is in the middle of the short peptide linker.

52. The polypeptide of claim 49, wherein the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS, GGGSGGS (SEQ ID NO: 78), and GGGSEGGGS (SEQ ID NO: 72).

53. A polypeptide comprising an interleukin-21 (IL-21) variant, wherein the amino acid segment of STNAGRRQKHR (SEQ ID NO: 71) at positions 80-90 corresponding to a wildtype human IL-21 is substituted with a short peptide linker.

54. The polypeptide of claim 53, wherein the length of the short peptide linker is from 4 to 10 or from 5 to 9 amino acid residues.

55. The polypeptide of claim 53 or 54, wherein the short peptide linker comprises one or more Gly-Ser units, and optionally one glutamic acid residue.

56. The polypeptide of claim 55, wherein the short peptide linker comprises the glutamic acid residue, and the glutamic acid residue is in the middle of the short peptide linker.

57. The polypeptide of claim 54, wherein the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS, GGGSGGS (SEQ ID NO: 78), and GGGSEGGGS (SEQ ID NO: 72).

58. The polypeptide of any one of claims 53-57, wherein the polypeptide comprises one or more amino acid residue substitutions at positions selected from the group consisting of R5, H6, R9, Q12, L13, D15, I16, S70, K72, K73, R76, P78, and P79.

59. The polypeptide of claim 58, wherein the polypeptide comprises the amino acid residue substitution at position R5, and optionally the substitution is R5C.

60. The polypeptide of claim 58, wherein the polypeptide comprises the amino acid residue substitution at position H6, and optionally the substitution is H6C.

61. The polypeptide of claim 58, wherein the polypeptide comprises the amino acid residue substitution at position R9, and optionally the substitution is R9C.

62. The polypeptide of claim 58, wherein the polypeptide comprises the amino acid residue substitution at position Q12, and optionally the substitution is Q12W.

63. The polypeptide of claim 58, wherein the polypeptide comprises the amino acid residue substitution at position L13, and optionally the substitution is L13A.

64. The polypeptide of claim 58, wherein the polypeptide comprises the amino acid residue substitution at position D15, and optionally the substitution is D15L, DISK, or D1SR.

65. The polypeptide of claim 58, wherein the polypeptide comprises the amino acid residue substitution at position 116, and optionally the substitution is 116R or I16W.

66. The polypeptide of claim 58, wherein the polypeptide comprises the amino acid residue substitution at position 570, and optionally the substitution is S70L or S70A.

67. The polypeptide of claim 58, wherein the polypeptide comprises the amino acid residue substitution at position K72, and optionally the substitution is K72S.

68. The polypeptide of claim 58, wherein the polypeptide comprises the amino acid residue substitution at position K73, and optionally the substitution is K73Y or K73F.

69. The polypeptide of claim 58, wherein the polypeptide comprises the amino acid residue substitution at position R76, and optionally the substitution is R76F.

70. The polypeptide of claim 58, wherein the polypeptide comprises the amino acid residue substitution at position P78, and optionally the substitution is P78G or P78E.

71. The polypeptide of claim 58, wherein the polypeptide comprises the amino acid residue substitution at position P79, and optionally the substitution is P79E or P79C.

72. A polypeptide comprising an interleukin-21 (IL-21) variant comprising a wildtype human IL-21 amino acid sequence with at least one mutation selected from RSC, H6C, R9C, Q12W, L13A, D15X1, I16X2, S70X3, K72S, K73X4, R76F, P78X5, or P79X6, wherein X1 is L, K, or R, wherein X2 is R or W, wherein X3 is L or A, wherein X4 is Y or F, and X5 is G or E, and wherein X6 is E or C.

73. The polypeptide of claim 72, wherein the wildtype human IL-21 comprises a sequence as set forth in SEQ ID NO: 1 or 2.

74. The polypeptide of any one of claims 72-73, wherein the amino acid segment of STNAGRRQKHR (SEQ ID NO: 71) at positions 80-90 corresponding to the wildtype human IL-21 is substituted with a short peptide linker.

75. The polypeptide of claim 74, wherein the length of the short peptide linker is from 4 to 10 or from 5 to 9 amino acid residues.

76. The polypeptide of claims 74 or 75, wherein the short peptide linker comprises one or more Gly-Ser units, and optionally one glutamic acid residue.

77. The polypeptide of claim 76, wherein the short peptide linker comprises the glutamic acid residue, and the glutamic acid residue is in the middle of the short peptide linker.

78. The polypeptide of claim 75, wherein the short peptide linker is selected from the group consisting of: GGSEGGS (SEQ ID NO: 73), GSEGS (SEQ ID NO: 74), GGSGGS (SEQ ID NO: 75), GGGSEGGS (SEQ ID NO: 76), GGSEGGGS (SEQ ID NO: 77), GSGGS (SEQ ID NO: 78), GGGSGGS (SEQ ID NO: 79), and GGGSEGGGS (SEQ ID NO: 72).

79. A polypeptide comprising an interleukin-21 (IL-21) variant, wherein the polypeptide comprises amino acid residue mutations selected from the group consisting of 1) P79E;
1) K73Y, P79E;
2) K73F, P79E;
3) S70L, K73F, P79E;
4) S70L, K73Y, P79E;
5) S70L, K72S, K73F, P79E;
6) S70L, K72S, K73Y, P79E;
7) D15K, S70L, K73F, P79E;
8) D15R, S70L, K73F, P79E;
9) I16W, S70L, K73F, P79E;
10) D15L, S70L, K73F, P79E;
11) L13A, S70L, K73F, P79E;
12) D15R, R76F, P78E, P79E;
13) D15L, I16R, S70L, K73Y, P79E;
14) D15K, I16W, S70L, R76F, P79E;
15) D15R, 116W, S70L, R76F, P79E;
16) STNAGRRQKHR (SEQ ID NO: 71) substituted with GGGSEGGGS (SEQ ID NO: 72) (STNAGRRQKHR (SEQ ID NO: 71)/GGGSEGGGS (SEQ ID NO: 72));
17) P79E, STNAGRRQKHR (SEQ ID NO: 71)/GGGSEGGGS (SEQ ID NO: 72);
18) K73Y, P79E, STNAGRRQKHR (SEQ ID NO: 71)/GGGSEGGGS (SEQ ID NO: 72);
19) K73F, P79E, STNAGRRQKHR (SEQ ID NO: 71)/GGGSEGGGS (SEQ ID NO: 72);
20) S70L, K73F, P79E, STNAGRRQKHR (SEQ ID NO: 71)/GGGSEGGGS (SEQ ID NO: 72);
21) S70L, K73Y, P79E, STNAGRRQKHR (SEQ ID NO: 71)/GGGSEGGGS (SEQ ID NO: 72);
22) D15K, S70L, K73F, P79E, STNAGRRQKHR (SEQ ID NO: 71)/GGGSEGGGS (SEQ ID NO: 72);
23) D15R, S70L, K73F, P79E, STNAGRRQKHR (SEQ ID NO: 71)/GGGSEGGGS (SEQ ID NO: 72);
24) 116W, S70L, K73F, P79E, STNAGRRQKHR (SEQ ID NO: 71)/GGGSEGGGS (SEQ ID NO: 72);
25) S70L, K72S, K73F, P79E, STNAGRRQKHR (SEQ ID NO: 71)/GGGSEGGGS (SEQ ID NO: 72);
26) S70L, K72S, K73Y, P79E, STNAGRRQKHR (SEQ ID NO: 71)/GGGSEGGGS (SEQ ID NO: 72);
27) D15R, 116W, S70L, K73Y, P79E, STNAGRRQKHR (SEQ ID NO: 71)/GGGSEGGGS (SEQ ID NO: 72); and
28) D15L, I16R, S70L, K73Y, P79E, STNAGRRQKHR (SEQ ID NO: 71)/GGGSEGGGS (SEQ ID NO: 72).

80. A polypeptide comprising an interleukin-21 (IL-21) variant, wherein the polypeptide comprises amino acid residue mutations selected from the group consisting of:
2) P79C;
3) R5C, P79C;
4) H6C, P79C;
5) R9C, P79C;
6) R5C, R76F, P79C;
7) H6C, R76F, P79C;
8) H6C, K73Y, P79C;
9) H6C, K73F, P79C;
10) H6C, S70L, K73Y, P79C;
11) H6C, S70L, K73F, P79C;
12) R9C, D15R, P78G, P79C;
13) H6C, D15L, Il6R, S70L, K73Y, P79C;
14) R9C, D15L, I16R, S70L, K73Y, P79C;
15) H6C, P79C, STNAGRRQKHR (SEQ ID NO: 71)/GGGSEGGGS (SEQ ID NO: 72);
16) H6C, K73Y, P79C, STNAGRRQKHR (SEQ ID NO: 71)/GGGSEGGGS (SEQ ID NO: 72);
17) H6C, K73F, P79C, STNAGRRQKHR (SEQ ID NO: 71)/GGGSEGGGS (SEQ ID NO: 72); and
18) H6C, S70L, P79C, STNAGRRQKHR (SEQ ID NO: 71)/GGGSEGGGS (SEQ ID NO: 72).

81. A polypeptide comprising an interleukin-21 (IL-21) variant, wherein the polypeptide comprises amino acid residue mutations selected from the group consisting of:
1) K73Y;
2) K73F;
3) S70L, K73Y;
4) S70L, K73F;
5) S70L, K72S, K73Y;
6) S70A, K72S, K73F;
7) Q12W, S70A, R76F;
8) D15K, S70A, R76F;
9) D15L, S70A, R76F;
10) D15R, S70A, R76F;
11) I16R, S70L, K73Y;
12) D15L, I16R, S70L, K73Y;
13) D15K, I16W, S70L, K73Y;
14) D15K, I16W, S70L, R76F;
15) D15R, I16W, S70L, R76F;
16) 116W, S70L, K72S, K73F;
17) D15R, I16W, S70L, K73Y; and
18) D15K, I16W, S70L, K72S, K73Y.

82. A polypeptide comprising an interleukin-21 (IL-21) variant, wherein the polypeptide comprises amino acid residue mutations selected from the group consisting of: S70L, K73F, P79E; S70L, K72S, K73F, P79E; S70L, K73Y, P79E; and S70L, K72S, K73Y, P79E.

83. A polypeptide comprising an interleukin-21 (IL-21) variant, wherein the polypeptide comprises a sequence as set forth in any one of SEQ ID Nos. 5-62.

84. A polypeptide comprising an interleukin-21 (IL-21) variant, wherein the polypeptide consists of a sequence as set forth in any one of SEQ ID Nos. 5-62.

85. The polypeptide of any one of the preceding claims, wherein the polypeptide exhibits a lower binding affinity for IL-21 receptor (IL-21R) than the wildtype human IL-21.

86. The polypeptide of claim 85, wherein the polypeptide exhibits 2 to 1000 fold reduction in binding affinity for IL-21R as compared to the wildtype human IL-21.

87. The polypeptide of claim 86, wherein the polypeptide has a higher K_{D} value for IL-21R as compared to the wildtype human IL-21.

88. A conjugate comprising the polypeptide of any one of the preceding claims and at least one additional moiety.

89. The conjugate of claim 88, wherein the at least one additional moiety comprises a crystallizable fragment (Fe) domain of an antibody.

90. The conjugate of claim 89, wherein the antibody is IgG, IgA, IgD, IgM, or IgE.

91. The conjugate of claim 90, wherein the IgG is IgG1, IgG2, IgG3, or IgG4.

92. The conjugate of any one of claims 88-91, wherein the at least one additional moiety comprises an antigen-binding molecule.

93. The conjugate of claim 92, wherein the antigen-binding molecule is an antibody or an antigen binding fragment thereof.

94. The conjugate of claim 93, wherein the antigen-binding molecule is a multi-specific antigen-binding molecule.

95. The conjugate of any one of claims 92-94, wherein the antigen-binding molecule targets a tumor cell or an immune cell.

96. The conjugate of claim 95, wherein the antigen is a tumor-associated antigen.

97. The conjugate of claim 95, wherein the antigen is an immune-checkpoint antigen or an immune-checkpoint-associated antigen.

98. The conjugate of claim 97, wherein the antigen is involved in an immune checkpoint pathway, and optionally the antigen is selected from the group consisting of: PD-1, PD-L1, TIGIT, CTLA-4, PD-L2, B7-H3, B7-H4, BTLA, LAG3, CD112, CD112R, CD96, TIM-3, CD47, and CEACAM1.

99. The conjugate of claim 97, wherein the antigen is a co-stimulatory immune checkpoint target, and optionally the antigen is selected from the group consisting of CD155, ICOS, OX40, CD137, CD137L, CD27, CD28, and GITR.

100. The conjugate of any one of claims 88-99, wherein the at least one additional moiety is attached to C-terminus and/or N-terminus of the IL-21 variant.

101. The conjugate of claim 100, wherein the at least one additional moiety is attached to the IL-21 variant directly or via a linker.

102. The polypeptide or the conjugate of any one of preceding claims, wherein the polypeptide exhibits an improved thermostability as compared to the wildtype human IL-21.

103. A nucleic acid molecule encoding the polypeptide of any one of claims 1-87 or the conjugate of any one of claims 88-101.

104. A vector comprising the nucleic acid molecule of claim 103.

105. The vector of claim 104, wherein the vector comprises a viral vector.

106. A transformed or host cell that expresses the polypeptide of any one of claims 1-87 or the conjugate of any one of claims 88-101.

107. A pharmaceutical composition comprising the polypeptide of any one of claims 1-87 or the conjugate of any one of claims 88-101, and a pharmaceutically acceptable carrier.

108. A kit comprising the polypeptide, the conjugate, the nucleic acid molecule, the vector, the transformed or host cell, or the pharmaceutical composition, a combination thereof, of any one of the preceding claims, and a container.

109. A method of preparing the polypeptide of 1-87 or the conjugate of claims 88-101, comprising:
a) constructing the nucleic acid molecule of claim 103 and the vector of claim 104 or 105;
b) culturing the transformed or host cell of claim 106; and
c) harvesting the polypeptide from the transformed or host cell.

110. A method of treating a subject in need thereof, comprising administering to the subject the pharmaceutical composition of claim 107 in an amount effective to treat the subject.

111. The method of claim 110, wherein the subject has or is suspected to have a solid tumor.

112. The nucleic acid molecule of claim 103, wherein the nucleic acid molecule is a DNA molecule.

113. The nucleic acid molecule of claim 103, wherein the nucleic acid molecule is a RNA molecule.

114. The nucleic acid molecule of claim 113, wherein the nucleic acid molecule is an mRNA molecule.


SEQUENCE LISTING (94 sequences)

SEQ ID NO: 1 - protein, 133 aa, Homo sapiens [sequence omitted]

SEQ ID NO: 2 - protein, 124 aa, Homo sapiens
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 3 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 4 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 5 - protein, 133 aa, synthetic construct
QGQDRHMIRMRQLIDIVDQLKNYVNDLVPEFLPAPEDVETNCEWSAFSCFQKAQLKSANTGNNERIINVSIKKLKRKPESTNAGRRQKHRLTCPSCDSYEKKPPKEFLERFKSLLQKMIHQHLSSRTHGSEDS

SEQ ID NO: 6 - protein, 131 aa, synthetic construct
QGQDRHMIRMRQLIDIVDQLKNYVNDLVPEFLPAPEDVETNCEWSAFSCFQKAQLKSANTGNNERIINVSIKKLKRKPPGGGSEGGGSLTCPSCDSYEKKPPKEFLERFKSLLQKMIHQHLSSRTHGSEDS

SEQ ID NO: 7 - protein, 133 aa, synthetic construct
QGQDCHMIRMRQLIDIVDQLKNYVNDLVPEFLPAPEDVETNCEWSAFSCFQKAQLKSANTGNNERIINVSIKKLKRKPCSTNAGRRQKHRLTCPSCDSYEKKPPKEFLERFKSLLQKMIHQHLSSRTHGSEDS

SEQ ID NO: 8 - protein, 133 aa, synthetic construct
QGQDRCMIRMRQLIDIVDQLKNYVNDLVPEFLPAPEDVETNCEWSAFSCFQKAQLKSANTGNNERIINVSIKKLKRKPCSTNAGRRQKHRLTCPSCDSYEKKPPKEFLERFKSLLQKMIHQHLSSRTHGSEDS

SEQ ID NO: 9 - protein, 131 aa, synthetic construct
QGQDRHMIRMRQLIDIVDQLKNYVNDLVPEFLPAPEDVETNCEWSAFSCFQKAQLKSANTGNNERIINVSIKKLKRKPEGGGSEGGGSLTCPSCDSYEKKPPKEFLERFKSLLQKMIHQHLSSRTHGSEDS

SEQ ID NO: 10 - protein, 131 aa, synthetic construct
QGQDRCMIRMRQLIDIVDQLKNYVNDLVPEFLPAPEDVETNCEWSAFSCFQKAQLKSANTGNNERIINVSIKKLKRKPCGGGSEGGGSLTCPSCDSYEKKPPKEFLERFKSLLQKMIHQHLSSRTHGSEDS

SEQ ID NO: 11 - protein, 133 aa, synthetic construct
QGQDRHMIRMRQLIDIVDQLKNYVNDLVPEFLPAPEDVETNCEWSAFSCFQKAQLKSANTGNNERIINVSIKYLKRKPPSTNAGRRQKHRLTCPSCDSYEKKPPKEFLERFKSLLQKMIHQHLSSRTHGSEDS

SEQ ID NO: 12 - protein, 133 aa, synthetic construct
QGQDRHMIRMRQLIDIVDQLKNYVNDLVPEFLPAPEDVETNCEWSAFSCFQKAQLKSANTGNNERIINVSIKYLKRKPESTNAGRRQKHRLTCPSCDSYEKKPPKEFLERFKSLLQKMIHQHLSSRTHGSEDS

SEQ ID NO: 13 - protein, 133 aa, synthetic construct
QGQDRCMIRMRQLIDIVDQLKNYVNDLVPEFLPAPEDVETNCEWSAFSCFQKAQLKSANTGNNERIINVSIKYLKRKPCSTNAGRRQKHRLTCPSCDSYEKKPPKEFLERFKSLLQKMIHQHLSSRTHGSEDS

SEQ ID NO: 14 - protein, 131 aa, synthetic construct
QGQDRHMIRMRQLIDIVDQLKNYVNDLVPEFLPAPEDVETNCEWSAFSCFQKAQLKSANTGNNERIINVSIKYLKRKPEGGGSEGGGSLTCPSCDSYEKKPPKEFLERFKSLLQKMIHQHLSSRTHGSEDS

SEQ ID NO: 15 - protein, 133 aa, synthetic construct
QGQDRHMIRMRQLIDIVDQLKNYVNDLVPEFLPAPEDVETNCEWSAFSCFQKAQLKSANTGNNERIINVSIKFLKRKPPSTNAGRRQKHRLTCPSCDSYEKKPPKEFLERFKSLLQKMIHQHLSSRTHGSEDS

SEQ ID NO: 16 - protein, 133 aa, synthetic construct
QGQDRHMIRMRQLIDIVDQLKNYVNDLVPEFLPAPEDVETNCEWSAFSCFQKAQLKSANTGNNERIINVSIKFLKRKPESTNAGRRQKHRLTCPSCDSYEKKPPKEFLERFKSLLQKMIHQHLSSRTHGSEDS

SEQ ID NO: 17 - protein, 133 aa, synthetic construct
QGQDRCMIRMRQLIDIVDQLKNYVNDLVPEFLPAPEDVETNCEWSAFSCFQKAQLKSANTGNNERIINVSIKFLKRKPCSTNAGRRQKHRLTCPSCDSYEKKPPKEFLERFKSLLQKMIHQHLSSRTHGSEDS

SEQ ID NO: 18 - protein, 131 aa, synthetic construct
QGQDRHMIRMRQLIDIVDQLKNYVNDLVPEFLPAPEDVETNCEWSAFSCFQKAQLKSANTGNNERIINVSIKFLKRKPEGGGSEGGGSLTCPSCDSYEKKPPKEFLERFKSLLQKMIHQHLSSRTHGSEDS

SEQ ID NO: 19 - protein, 133 aa, synthetic construct
QGQDRHMIRMRQLIDIVDQLKNYVNDLVPEFLPAPEDVETNCEWSAFSCFQKAQLKSANTGNNERIINVLIKYLKRKPPSTNAGRRQKHRLTCPSCDSYEKKPPKEFLERFKSLLQKMIHQHLSSRTHGSEDS

SEQ ID NO: 20 - protein, 133 aa, synthetic construct
QGQDRHMIRMRQLIDIVDQLKNYVNDLVPEFLPAPEDVETNCEWSAFSCFQKAQLKSANTGNNERIINVLIKYLKRKPESTNAGRRQKHRLTCPSCDSYEKKPPKEFLERFKSLLQKMIHQHLSSRTHGSEDS

SEQ ID NO: 21 - protein, 133 aa, synthetic construct
QGQDRCMIRMRQLIDIVDQLKNYVNDLVPEFLPAPEDVETNCEWSAFSCFQKAQLKSANTGNNERIINVLIKYLKRKPCSTNAGRRQKHRLTCPSCDSYEKKPPKEFLERFKSLLQKMIHQHLSSRTHGSEDS

SEQ ID NO: 22 - protein, 131 aa, synthetic construct
QGQDRHMIRMRQLIDIVDQLKNYVNDLVPEFLPAPEDVETNCEWSAFSCFQKAQLKSANTGNNERIINVLIKYLKRKPEGGGSEGGGSLTCPSCDSYEKKPPKEFLERFKSLLQKMIHQHLSSRTHGSEDS

SEQ ID NO: 23 - protein, 133 aa, synthetic construct
QGQDRHMIRMRQLIDIVDQLKNYVNDLVPEFLPAPEDVETNCEWSAFSCFQKAQLKSANTGNNERIINVLISYLKRKPPSTNAGRRQKHRLTCPSCDSYEKKPPKEFLERFKSLLQKMIHQHLSSRTHGSEDS

SEQ ID NO: 24 - protein, 133 aa, synthetic construct
QGQDRHMIRMRQLIDIVDQLKNYVNDLVPEFLPAPEDVETNCEWSAFSCFQKAQLKSANTGNNERIINVLISYLKRKPESTNAGRRQKHRLTCPSCDSYEKKPPKEFLERFKSLLQKMIHQHLSSRTHGSEDS

SEQ ID NO: 25 - protein, 131 aa, synthetic construct
QGQDRHMIRMRQLIDIVDQLKNYVNDLVPEFLPAPEDVETNCEWSAFSCFQKAQLKSANTGNNERIINVLISYLKRKPEGGGSEGGGSLTCPSCDSYEKKPPKEFLERFKSLLQKMIHQHLSSRTHGSEDS

SEQ ID NO: 26 - protein, 133 aa, synthetic construct
QGQDRHMIRMRQLIDRVDQLKNYVNDLVPEFLPAPEDVETNCEWSAFSCFQKAQLKSANTGNNERIINVLIKYLKRKPPSTNAGRRQKHRLTCPSCDSYEKKPPKEFLERFKSLLQKMIHQHLSSRTHGSEDS

SEQ ID NO: 27 - protein, 133 aa, synthetic construct
QGQDRHMIRMRQLILRVDQLKNYVNDLVPEFLPAPEDVETNCEWSAFSCFQKAQLKSANTGNNERIINVLIKYLKRKPPSTNAGRRQKHRLTCPSCDSYEKKPPKEFLERFKSLLQKMIHQHLSSRTHGSEDS

SEQ ID NO: 28 - protein, 133 aa, synthetic construct
QGQDRHMIRMRQLIKWVDQLKNYVNDLVPEFLPAPEDVETNCEWSAFSCFQKAQLKSANTGNNERIINVLIKYLKRKPPSTNAGRRQKHRLTCPSCDSYEKKPPKEFLERFKSLLQKMIHQHLSSRTHGSEDS

SEQ ID NO: 29 - protein, 133 aa, synthetic construct
QGQDRHMIRMRQLIRWVDQLKNYVNDLVPEFLPAPEDVETNCEWSAFSCFQKAQLKSANTGNNERIINVLIKYLKRKPPSTNAGRRQKHRLTCPSCDSYEKKPPKEFLERFKSLLQKMIHQHLSSRTHGSEDS

SEQ ID NO: 30 - protein, 133 aa, synthetic construct
QGQDRHMIRMRQLILRVDQLKNYVNDLVPEFLPAPEDVETNCEWSAFSCFQKAQLKSANTGNNERIINVLIKYLKRKPESTNAGRRQKHRLTCPSCDSYEKKPPKEFLERFKSLLQKMIHQHLSSRTHGSEDS

SEQ ID NO: 31 - protein, 131 aa, synthetic construct
QGQDRHMIRMRQLIRWVDQLKNYVNDLVPEFLPAPEDVETNCEWSAFSCFQKAQLKSANTGNNERIINVLIKYLKRKPEGGGSEGGGSLTCPSCDSYEKKPPKEFLERFKSLLQKMIHQHLSSRTHGSEDS

SEQ ID NO: 32 - protein, 133 aa, synthetic construct
QGQDRCMIRMRQLILRVDQLKNYVNDLVPEFLPAPEDVETNCEWSAFSCFQKAQLKSANTGNNERIINVLIKYLKRKPCSTNAGRRQKHRLTCPSCDSYEKKPPKEFLERFKSLLQKMIHQHLSSRTHGSEDS

SEQ ID NO: 33 - protein, 133 aa, synthetic construct
QGQDRHMICMRQLILRVDQLKNYVNDLVPEFLPAPEDVETNCEWSAFSCFQKAQLKSANTGNNERIINVLIKYLKRKPCSTNAGRRQKHRLTCPSCDSYEKKPPKEFLERFKSLLQKMIHQHLSSRTHGSEDS

SEQ ID NO: 34 - protein, 133 aa, synthetic construct
QGQDRHMIRMRQLIKWVDQLKNYVNDLVPEFLPAPEDVETNCEWSAFSCFQKAQLKSANTGNNERIINVLISYLKRKPPSTNAGRRQKHRLTCPSCDSYEKKPPKEFLERFKSLLQKMIHQHLSSRTHGSEDS

SEQ ID NO: 35 - protein, 131 aa, synthetic construct
QGQDRHMIRMRQLILRVDQLKNYVNDLVPEFLPAPEDVETNCEWSAFSCFQKAQLKSANTGNNERIINVLIKYLKRKPEGGGSEGGGSLTCPSCDSYEKKPPKEFLERFKSLLQKMIHQHLSSRTHGSEDS

SEQ ID NO: 36 - protein, 133 aa, synthetic construct
QGQDRHMIRMRQLIDIVDQLKNYVNDLVPEFLPAPEDVETNCEWSAFSCFQKAQLKSANTGNNERIINVLIKFLKRKPPSTNAGRRQKHRLTCPSCDSYEKKPPKEFLERFKSLLQKMIHQHLSSRTHGSEDS

SEQ ID NO: 37 - protein, 133 aa, synthetic construct
QGQDRHMIRMRQLIDIVDQLKNYVNDLVPEFLPAPEDVETNCEWSAFSCFQKAQLKSANTGNNERIINVLIKFLKRKPESTNAGRRQKHRLTCPSCDSYEKKPPKEFLERFKSLLQKMIHQHLSSRTHGSEDS

SEQ ID NO: 38 - protein, 133 aa, synthetic construct
QGQDRCMIRMRQLIDIVDQLKNYVNDLVPEFLPAPEDVETNCEWSAFSCFQKAQLKSANTGNNERIINVLIKFLKRKPCSTNAGRRQKHRLTCPSCDSYEKKPPKEFLERFKSLLQKMIHQHLSSRTHGSEDS

SEQ ID NO: 39 - protein, 131 aa, synthetic construct
QGQDRHMIRMRQLIDIVDQLKNYVNDLVPEFLPAPEDVETNCEWSAFSCFQKAQLKSANTGNNERIINVLIKFLKRKPEGGGSEGGGSLTCPSCDSYEKKPPKEFLERFKSLLQKMIHQHLSSRTHGSEDS

SEQ ID NO: 40 - protein, 133 aa, synthetic construct
QGQDRHMIRMRQLIDIVDQLKNYVNDLVPEFLPAPEDVETNCEWSAFSCFQKAQLKSANTGNNERIINVLISFLKRKPESTNAGRRQKHRLTCPSCDSYEKKPPKEFLERFKSLLQKMIHQHLSSRTHGSEDS

SEQ ID NO: 41 - protein, 131 aa, synthetic construct
QGQDRHMIRMRQLIDIVDQLKNYVNDLVPEFLPAPEDVETNCEWSAFSCFQKAQLKSANTGNNERIINVLISFLKRKPEGGGSEGGGSLTCPSCDSYEKKPPKEFLERFKSLLQKMIHQHLSSRTHGSEDS

SEQ ID NO: 42 - protein, 133 aa, synthetic construct
QGQDRHMIRMRQLIDIVDQLKNYVNDLVPEFLPAPEDVETNCEWSAFSCFQKAQLKSANTGNNERIINVAISFLKRKPPSTNAGRRQKHRLTCPSCDSYEKKPPKEFLERFKSLLQKMIHQHLSSRTHGSEDS

SEQ ID NO: 43 - protein, 131 aa, synthetic construct
QGQDRHMIRMRQLIKIVDQLKNYVNDLVPEFLPAPEDVETNCEWSAFSCFQKAQLKSANTGNNERIINVLIKFLKRKPEGGGSEGGGSLTCPSCDSYEKKPPKEFLERFKSLLQKMIHQHLSSRTHGSEDS

SEQ ID NO: 44 - protein, 131 aa, synthetic construct
QGQDRHMIRMRQLIRIVDQLKNYVNDLVPEFLPAPEDVETNCEWSAFSCFQKAQLKSANTGNNERIINVLIKFLKRKPEGGGSEGGGSLTCPSCDSYEKKPPKEFLERFKSLLQKMIHQHLSSRTHGSEDS

SEQ ID NO: 45 - protein, 131 aa, synthetic construct
QGQDRHMIRMRQLIDWVDQLKNYVNDLVPEFLPAPEDVETNCEWSAFSCFQKAQLKSANTGNNERIINVLIKFLKRKPEGGGSEGGGSLTCPSCDSYEKKPPKEFLERFKSLLQKMIHQHLSSRTHGSEDS

SEQ ID NO: 46 - protein, 133 aa, synthetic construct
QGQDRHMIRMRQAIDIVDQLKNYVNDLVPEFLPAPEDVETNCEWSAFSCFQKAQLKSANTGNNERIINVLIKFLKRKPESTNAGRRQKHRLTCPSCDSYEKKPPKEFLERFKSLLQKMIHQHLSSRTHGSEDS

SEQ ID NO: 47 - protein, 133 aa, synthetic construct
QGQDRHMIRMRQLIKIVDQLKNYVNDLVPEFLPAPEDVETNCEWSAFSCFQKAQLKSANTGNNERIINVLIKFLKRKPESTNAGRRQKHRLTCPSCDSYEKKPPKEFLERFKSLLQKMIHQHLSSRTHGSEDS

SEQ ID NO: 48 - protein, 133 aa, synthetic construct
QGQDRHMIRMRQLIRIVDQLKNYVNDLVPEFLPAPEDVETNCEWSAFSCFQKAQLKSANTGNNERIINVLIKFLKRKPESTNAGRRQKHRLTCPSCDSYEKKPPKEFLERFKSLLQKMIHQHLSSRTHGSEDS

SEQ ID NO: 49 - protein, 133 aa, synthetic construct
QGQDRHMIRMRQLIDWVDQLKNYVNDLVPEFLPAPEDVETNCEWSAFSCFQKAQLKSANTGNNERIINVLIKFLKRKPESTNAGRRQKHRLTCPSCDSYEKKPPKEFLERFKSLLQKMIHQHLSSRTHGSEDS

SEQ ID NO: 50 - protein, 133 aa, synthetic construct
QGQDRHMIRMRQLIDWVDQLKNYVNDLVPEFLPAPEDVETNCEWSAFSCFQKAQLKSANTGNNERIINVLISFLKRKPPSTNAGRRQKHRLTCPSCDSYEKKPPKEFLERFKSLLQKMIHQHLSSRTHGSEDS

SEQ ID NO: 51 - protein, 133 aa, synthetic construct
QGQDCHMIRMRQLIDIVDQLKNYVNDLVPEFLPAPEDVETNCEWSAFSCFQKAQLKSANTGNNERIINVSIKKLKFKPCSTNAGRRQKHRLTCPSCDSYEKKPPKEFLERFKSLLQKMIHQHLSSRTHGSEDS

SEQ ID NO: 52 - protein, 133 aa, synthetic construct
QGQDRCMIRMRQLIDIVDQLKNYVNDLVPEFLPAPEDVETNCEWSAFSCFQKAQLKSANTGNNERIINVSIKKLKFKPCSTNAGRRQKHRLTCPSCDSYEKKPPKEFLERFKSLLQKMIHQHLSSRTHGSEDS

SEQ ID NO: 53 - protein, 133 aa, synthetic construct
QGQDRHMIRMRQLIKWVDQLKNYVNDLVPEFLPAPEDVETNCEWSAFSCFQKAQLKSANTGNNERIINVLIKKLKFKPPSTNAGRRQKHRLTCPSCDSYEKKPPKEFLERFKSLLQKMIHQHLSSRTHGSEDS

SEQ ID NO: 54 - protein, 133 aa, synthetic construct
QGQDRHMIRMRQLIRWVDQLKNYVNDLVPEFLPAPEDVETNCEWSAFSCFQKAQLKSANTGNNERIINVLIKKLKFKPPSTNAGRRQKHRLTCPSCDSYEKKPPKEFLERFKSLLQKMIHQHLSSRTHGSEDS

SEQ ID NO: 55 - protein, 133 aa, synthetic construct
QGQDRHMICMRQLIRIVDQLKNYVNDLVPEFLPAPEDVETNCEWSAFSCFQKAQLKSANTGNNERIINVSIKKLKRKGCSTNAGRRQKHRLTCPSCDSYEKKPPKEFLERFKSLLQKMIHQHLSSRTHGSEDS

SEQ ID NO: 56 - protein, 133 aa, synthetic construct
QGQDRHMIRMRQLIKWVDQLKNYVNDLVPEFLPAPEDVETNCEWSAFSCFQKAQLKSANTGNNERIINVLIKKLKFKPESTNAGRRQKHRLTCPSCDSYEKKPPKEFLERFKSLLQKMIHQHLSSRTHGSEDS

SEQ ID NO: 57 - protein, 133 aa, synthetic construct
QGQDRHMIRMRQLIRWVDQLKNYVNDLVPEFLPAPEDVETNCEWSAFSCFQKAQLKSANTGNNERIINVLIKKLKFKPESTNAGRRQKHRLTCPSCDSYEKKPPKEFLERFKSLLQKMIHQHLSSRTHGSEDS

SEQ ID NO: 58 - protein, 133 aa, synthetic construct
QGQDRHMIRMRQLIRIVDQLKNYVNDLVPEFLPAPEDVETNCEWSAFSCFQKAQLKSANTGNNERIINVSIKKLKFKEESTNAGRRQKHRLTCPSCDSYEKKPPKEFLERFKSLLQKMIHQHLSSRTHGSEDS

SEQ ID NO: 59 - protein, 133 aa, synthetic construct
QGQDRHMIRMRWLIDIVDQLKNYVNDLVPEFLPAPEDVETNCEWSAFSCFQKAQLKSANTGNNERIINVAIKKLKFKPPSTNAGRRQKHRLTCPSCDSYEKKPPKEFLERFKSLLQKMIHQHLSSRTHGSEDS

SEQ ID NO: 60 - protein, 133 aa, synthetic construct
QGQDRHMIRMRQLIKIVDQLKNYVNDLVPEFLPAPEDVETNCEWSAFSCFQKAQLKSANTGNNERIINVAIKKLKFKPPSTNAGRRQKHRLTCPSCDSYEKKPPKEFLERFKSLLQKMIHQHLSSRTHGSEDS

SEQ ID NO: 61 - protein, 133 aa, synthetic construct
QGQDRHMIRMRQLILIVDQLKNYVNDLVPEFLPAPEDVETNCEWSAFSCFQKAQLKSANTGNNERIINVAIKKLKFKPPSTNAGRRQKHRLTCPSCDSYEKKPPKEFLERFKSLLQKMIHQHLSSRTHGSEDS

SEQ ID NO: 62 - protein, 133 aa, synthetic construct
QGQDRHMIRMRQLIRIVDQLKNYVNDLVPEFLPAPEDVETNCEWSAFSCFQKAQLKSANTGNNERIINVAIKKLKFKPPSTNAGRRQKHRLTCPSCDSYEKKPPKEFLERFKSLLQKMIHQHLSSRTHGSEDS

SEQ ID NO: 63 - protein, 232 aa, Homo sapiens
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 18 aa, synthetic construct
MHSSALLCCLVLLTGVRA

SEQ ID NO: 65 - protein, 383 aa, synthetic construct
MHSSALLCCLVLLTGVRAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - DNA, 1197 bp, synthetic construct
  source 1..1197: mol_type = other DNA
gcggccgcaaactacaagacagacttgcaaaagaaggcatgcacagctcagcactgctctgttgcctggtcctcctgactggggtgagggcccagggacaagacagacacatgatcagaatgagacaactgatcgacatagtggaccaactcaaaaactacgtcaacgacctcgtccccgagttcctccccgccccagaggacgtcgagaccaactgcgaatggagcgccttcagctgcttccagaaagcccagctcaagtccgccaacaccggcaacaacgaaagaatcatcaacgtgagcatcaaaaagctgaagagaaaacccccctccaccaacgccggcagaagacagaagcacagactgacctgcccctcctgcgacagctacgaaaaaaagccacccaaggagttcctcgaaagattcaaatccctgctccaaaaaatgatccaccaacacctcagcagcagaacccacggctccgaagactccgagcccaaatctgctgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgattctaga

SEQ ID NO: 67 - protein, 144 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSHHHHHH

SEQ ID NO: 68 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 380 aa, synthetic construct
MHSSALLCCLVLLTGVRAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 70 - protein, 362 aa, synthetic construct
QGQDRHMIRMRQLIDIVDQLKNYVNDLVPEFLPAPEDVETNCEWSAFSCFQKAQLKSANTGNNERIINVLISFLKRKPESTNAGRRQKHRLTCPSCDSYEKKPPKEFLERFKSLLQKMIHQHLSSRTHGSEDSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 11 aa, Homo sapiens
STNAGRRQKHR

SEQ ID NO: 72 - protein, 9 aa, synthetic construct
GGGSEGGGS

SEQ ID NO: 73 - protein, 7 aa, synthetic construct
GGSEGGS

SEQ ID NO: 74 - protein, 5 aa, synthetic construct
GSEGS

SEQ ID NO: 75 - protein, 6 aa, synthetic construct
GGSGGS

SEQ ID NO: 76 - protein, 8 aa, synthetic construct
GGGSEGGS

SEQ ID NO: 77 - protein, 8 aa, synthetic construct
GGSEGGGS

SEQ ID NO: 78 - protein, 5 aa, synthetic construct
GSGGS

SEQ ID NO: 79 - protein, 7 aa, synthetic construct
GGGSGGS

SEQ ID NO: 80 - protein, 10 aa, synthetic construct
  VARIANT 6..10: note = Residue may be deleted
HHHHHHHHHH

SEQ ID NO: 81 - protein, 6 aa, synthetic construct
HHHHHH

SEQ ID NO: 82 - protein, 8 aa, synthetic construct
HHHHHHHH

SEQ ID NO: 83 - protein, 13 aa, synthetic construct
PPSTNAGRRQKHR

SEQ ID NO: 84 - protein, 13 aa, synthetic construct
PESTNAGRRQKHR

SEQ ID NO: 85 - protein, 11 aa, synthetic construct
PPGGGSEGGGS

SEQ ID NO: 86 - protein, 13 aa, synthetic construct
PCSTNAGRRQKHR

SEQ ID NO: 87 - protein, 11 aa, synthetic construct
PEGGGSEGGGS

SEQ ID NO: 88 - protein, 11 aa, synthetic construct
PCGGGSEGGGS

SEQ ID NO: 89 - protein, 13 aa, synthetic construct
GCSTNAGRRQKHR

SEQ ID NO: 90 - protein, 13 aa, synthetic construct
EESTNAGRRQKHR

SEQ ID NO: 91 - protein, 5 aa, synthetic construct
GGGGS

SEQ ID NO: 92 - protein, 443 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 93 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 94 - protein, 581 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGSQGQDRHMIRMRQLIDIVDQLKNYVNDLVPEFLPAPEDVETNCEWSAFSCFQKAQLKSANTGNNERIINVLISYLKRKPESTNAGRRQKHRLTCPSCDSYEKKPPKEFLERFKSLLQKMIHQHLSSRTHGSEDS